(19) United States
(12) Patent Application Publication
(10) Pub. No.: US 2023/0310521 A1
(43) Pub. Date: Oct. 5, 2023

(54) COMBINATION COMPRISING BACILLUS AND AN ESSENTIAL OIL AND METHODS FOR MAKING AND USING

(71) Applicant: Phibro Animal Health Corporation, Teaneck, NJ (US)

(72) Inventor: A. Bruce Johnson, Teaneck, NJ (US)

(73) Assignee: Phibro Animal Health Corporation, Teaneck, NJ (US)

(21) Appl. No.: 18/191,328

(22) Filed: Mar. 28, 2023

Related U.S. Application Data
(60) Provisional application No. 63/326,690, filed on Apr. 1, 2022.

(51) Int. Cl. A61K 35/742; A61K 36/88; A61K 36/185; A61K 36/53; A61K 9/00; A61K 45/06; A61P 39/00; A61P 31/04; A23K 10/18; A23K 10/30; A23K 20/158; A23K 50/75

(52) U.S. Cl. CPC A61K 35/742; A61K 36/88; A61K 36/185; A61K 36/53; A61K 9/0056; A61K 45/06; A61P 39/00; A61P 31/04; A23K 10/18; A23K 10/30; A23K 20/158; A23K 50/75

(57) ABSTRACT

Disclosed herein are embodiments of a combination and/or composition comprising a Bacillus composition comprising three or four direct fed microbial (DFMs) selected from Bacillus coagulans, Bacillus subtilis, Bacillus licheniformis and Bacillus amyloliquefaciens, and an essential oil composition, and optionally may further comprise yucca and/or quillaja, such as Yucca schidigera and/or Quillaja saponaria. The essential oil composition may comprise oregano essential oil. Also disclosed are embodiments of a feed composition comprising the disclosed combination and/or composition and a feed. Also methods for administering the combination and/or composition to a non-ruminant animal, particularly an avian, are disclosed. The combination and/or composition may provide a beneficial effect to the animal upon administration, such as, but not limited to, weight gain, feed conversion, and/or reducing bacterial load.

### CROSS REFERENCE TO RELATED APPLICATION

This application claims the benefit of the earlier filing date of U.S. Provisional Application No. 63/326,690, filed on Apr. 1, 2022, which is incorporated herein by reference in its entirety.

### FIELD

The present application concerns a combination and/or composition comprising a Bacillus combination and an essential oil, and may further comprise yucca and/or quillaja, or extracts thereof, and a method administering the combination and/or composition to animals, particularly avians.

### SUMMARY

Disclosed herein are embodiments of a combination and/or composition comprising a Bacillus combination and an essential oil composition. The combination and/or composition may further comprise yucca and/or quillaja. The Bacillus combination may comprise, consist essentially of, or consist of, Bacillus amyloliquefaciens, Bacillus subtilis, Bacillus licheniformis and Bacillus coagulans. In some embodiments, the Bacillus combination consists essentially of from 30% to 45% Bacillus subtilis, from 40% to 60% Bacillus licheniformis, from 10% to 25% Bacillus amyloliquefaciens and from 1% to 12% Bacillus coagulans. And/or the Bacillus combination may further comprise a carrier, such as limestone (calcium carbonate).

The essential oil composition may comprise, consist essentially of, or consist of, an essential oil, such as oregano essential oil. In some aspects, the oregano essential oil comprises carvacrol and thymol, and may comprise carvacrol, thymol, γ-terpinene, p-cymene, and β-caryophyllene.

The combination and/or composition may further comprise yucca and/or quillaja, such as Yucca schidigera and/or Quillaja saponaria, or extracts thereof.

In any embodiments, the combination and/or composition may further comprise an additional component, such as silica, mineral clay, glucan, mannans, endoglucanohydrolase, a copper salt, a vitamin, a chromium compound, metal chelate, growth factor, allicin, alliin, alliinase, yeast, DFM, or a combination thereof.

Also disclosed herein are embodiments of a feed composition comprising the combination and/or composition disclosed herein, and an animal feed, such as a poultry feed. The feed composition may comprise from 1×10⁵ to 1×10⁷ CFU of the Bacillus combination per gram of the feed, such as from 2.5×10⁵ to 5×10⁶ CFU of the Bacillus combination per gram of the feed. In other embodiments, the feed composition comprises from 0.1 pounds to 1 pound of the Bacillus combination per ton of the feed, such as from 0.2 pounds to 0.3 pounds of the Bacillus combination per ton of the feed.

The feed composition may comprise the essential oil composition in an amount sufficient to provide from 25 ppm to 500 ppm of essential oil, such as from 50 ppm to 250 ppm of essential oil, such as oregano essential oil.

In a particular embodiment, the feed composition comprises 0.25 pounds of the Bacillus combination per ton of feed and 50 ppm of essential oil per ton of feed.

In an alternative embodiment, the feed composition comprises the Bacillus combination in an amount sufficient to provide 1×10⁶ CFU Bacillus per gram of feed and 50 ppm of essential oil per ton of feed.

In any embodiments, the feed composition may further comprise from 100 ppm to 500 ppm Yucca schidigera and Quillaja Saponaria or extracts thereof per ton of feed, such as from 200 ppm to 400 ppm, or 350 ppm per ton of feed.

Also disclosed herein is a method of administering the disclosed combination and/or composition, or a feed composition comprising the disclosed combination and/or composition, to a non-ruminant animal. The animal may be poultry, and/or administration of the combination and/or composition, or the feed composition comprising the combination and/or composition, increases breast meat yield, increases weight gain, improves a feed conversion ratio, reduces bird mortality, reduces lesion scores, reduces Salmonella/E. Coli/Clostridium perfringens (CP) incidence, reduces oocysts in fecal matter, or a combination thereof.

The foregoing and other objects, features, and advantages of the invention will become more apparent from the following detailed description, which proceeds with reference to the accompanying figures.

### BRIEF DESCRIPTION OF THE DRAWINGS

FIG. 1 is a table illustrating the treatment groups from the study described in Example 1.

FIG. 2 is a table illustrating results from days 1-14 of the study described in Example 1.

FIG. 3 is a table illustrating results from days 1-21 and days 1-35 of the study described in Example 1.

FIG. 4 is a table illustrating results from days 1-42 and days 15-22 of the study described in Example 1.

FIG. 5 is a table illustrating results from days 22-35 and days 36-42 of the study described in Example 1.

FIG. 6 is a table illustrating water consumption per bird and lesion scores from the study described in Example 1.

FIG. 7 is a table illustrating intestinal bacteria data from the study described in Example 1.

FIG. 8 is a table illustrating villi cell measurements from the study described in Example 1.

FIG. 9 is a table illustrating the treatment groups from the study described in Example 3.

FIG. 10 is a table illustrating results for treatment groups 1-13 from days 1-14 of the study described in Example 3.

FIG. 11 is a table illustrating results for treatment groups 14-18 from days 1-14 of the study described in Example 3.

FIG. 12 is a table illustrating results for treatment groups 1-13 from days 1-21 and days 1-35 of the study described in Example 3.

FIG. 13 is a table illustrating results for treatment groups 14-18 from days 1-21 and days 1-35 of the study described in Example 3.

FIG. 14 is a table illustrating results for treatment groups 1-13 from days 1-42 and days 15-22 of the study described in Example 3.

FIG. 15 is a table illustrating results for treatment groups 14-18 from days 1-42 and days 15-22 of the study described in Example 3.

FIG. 16 is a table illustrating results for treatment groups 1-13 from days 22-35 and days 36-42 of the study described in Example 3.

FIG. 17 is a table illustrating results for treatment groups 14-18 from days 22-35 and days 36-42 of the study described in Example 3.

FIG. 18 is a table illustrating water consumption per bird and lesion scores for treatment groups 1-13 from the study described in Example 3.

FIG. 19 is a table illustrating water consumption per bird and lesion scores for treatment groups 14-18 from the study described in Example 3.

FIG. 20 is a table illustrating intestinal bacteria data and villi cell measurements for treatment groups 1-13 from the study described in Example 3.

FIG. 21 is a table illustrating intestinal bacteria data and villi cell measurements for treatment groups 14-18 from the study described in Example 3.

### DETAILED DESCRIPTION

### I. Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “of” refers to a single element of stated alternative elements or a combination of two or more elements, unless the context clearly indicates otherwise.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Administering: Administration by any route to a subject, such as poultry. In some embodiments, the route of administration is oral.

Aliphatic: A substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀) from one to six (C₁₋₆), or from one to four carbon atoms (C₁₋₄) for an acyclic aliphatic group or moiety. A person of ordinary skill in the art will understand that for an alkenyl or alkynyl moiety, the minimum number of carbon atoms is two. And for a cyclic aliphatic group or moiety, the number of carbon atoms must be at least three, such as from three to fifteen (C₃₋₁₅) from three to ten (C₃₋₁₀), from three to six (C₃₋₆), or from three to four (C₃₋₄) carbon atoms. An aliphatic group may be substituted or unsubstituted, unless expressly referred to as an “unsubstituted aliphatic” or a “substituted aliphatic.” An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group).

Aromatic: A cyclic, conjugated group or moiety of, unless specified otherwise, from 5 to 15 ring atoms having a single ring (e.g., phenyl, or pyridinyl) or multiple condensed rings in which at least one ring is aromatic (e.g., indolyl), that is at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

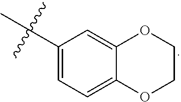 [SMILES: *c1ccc2c(c1)OCCO2]

However, in certain examples, context or express disclosure may indicate that the point of attachment is through a non-aromatic portion of the condensed ring system. For example,

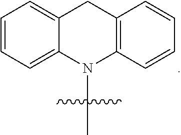 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Unless otherwise stated, an aromatic group may be substituted or unsubstituted.

Aryl: An aromatic carbocyclic group of, unless specified otherwise, from 6 to 15 carbon atoms having a single ring (e.g., phenyl) or multiple condensed rings in which at least one ring is aromatic (e.g., benzodioxolyl). If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

Amino acids: An organic acid containing both an amino group (such as —NH₂) and a carboxylic acid group (—COOH). The proteinogenic amino acids are α-amino acids, i.e., both the amine moiety and the —COOH moiety are attached to the same carbon.

Antimicrobial: An agent that kills and/or inhibits the growth of microorganisms. As used herein, antimicrobials include antibiotics, antifungals, antivirals, and antiparasitics including anticoccidials, or combinations thereof.

Carrier: A physiologically inert substance that is used as an additive in (or with) a combination, composition, or component as disclosed herein. As used herein, a carrier may be incorporated within particles of a combination, composition, or component, or it may be physically mixed with particles of a combination, composition, or component. A carrier can be used, for example, to dilute an active agent and/or to modify properties of a combination or composition, such as flowability, stability during storage, exposure to moisture, etc. Examples of carriers include, but are not limited to, calcium carbonate, polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Chelate: A chemical complex comprising at least one metal ion and at least one ligand.

Chelator: A compound that can bind to a metal ion to form a metal chelate. Once bound to the metal ion, the chelator typically referred to as a ligand. The chemical structure of the chelator may be different after it is bound to the metal ion. For example, the ligand may be deprotonated compared to the chelator, such as at a carboxylate and/or O⁻ moiety. Examples of chelators may include, but are not limited to, amino acids and alpha and beta-hydroxy acids.

Colony forming units (CFU): “Colony forming units” refers to individual colonies of bacteria. A colony is a mass of individual bacteria growing together. For certain embodiments, a colony comprises substantially the same species, and may comprise, but does not necessarily comprise, substantially the same strain. CFU are a measure of the number of bacteria present in or on a surface of a sample. However, CFU is not necessarily a measure of individual cells or spores, as a colony may be formed from a single or a mass of cells or spores.

Combination: A combination includes two or more components that are administered such that the effective time period of at least one component overlaps with the effective time period of at least one other component. A combination, or a component thereof, may be a composition. In some embodiments, effective time periods of all components administered overlap with each other. In an exemplary embodiment of a combination comprising three components, the effective time period of the first component administered may overlap with the effective time periods of the second and third components, but the effective time periods of the second and third components independently may or may not overlap with one another. In another exemplary embodiment of a combination comprising three components, the effective time period of the first component administered overlaps with the effective time period of the second component, but not that of the third component; and the effective time period of the second component overlaps with those of the first and third components. A combination may be a composition comprising the components, a composition comprising one or more components and another separate component (or components) or composition(s) comprising the remaining component(s), or the combination may be two or more individual components. In some embodiments, the two or more components may comprise the same component administered at two or more different times, two or more different components administered substantially simultaneously or sequentially in any order, or a combination thereof.

Bacillus combination: Refers to a combination, or a composition, of DFMs including only three or four Bacillus species selected from Bacillus amyloliquefaciens, Bacillus subtilis, Bacillus licheniformis and Bacillus coagulans. In some disclosed embodiments, “Bacillus combination” refers to a composition for administration to a subject, particularly to a non-ruminant animal, and even more particularly to an avian, such as chickens and turkeys, that consists of or consists essentially of any three or four of Bacillus amyloliquefaciens, Bacillus subtilis, Bacillus licheniformis and Bacillus coagulans. In other embodiments, “Bacillus combination” refers to Bacillus amyloliquefaciens, Bacillus subtilis, Bacillus licheniformis and Bacillus coagulans administered in combination without any other DFMs. A person of ordinary skill in the art will understand that the Bacillus combination may include additional residual material that is carried over from the production of any or all of the three or four Bacillus species, such as a dry milk product, and/or a carrier that does not materially affect the structure, function, novel and/or basic features of the Bacillus species.

CSL Combination: Refers to a combination, or a composition, of DFMs including only Bacillus coagulans, Bacillus subtilis and Bacillus licheniformis. In some disclosed embodiments, “CSL combination” refers to a composition for administration to a subject, particularly to an animal, and even more particularly to an avian, such as chickens and turkeys, that consists of or consists essentially of Bacillus coagulans, Bacillus subtilis and Bacillus licheniformis. In other embodiments, “CSL combination” refers to Bacillus coagulans, Bacillus subtilis and Bacillus licheniformis administered in combination without any other DFMs. A person of ordinary skill in the art will understand that the CSL combination may include additional residual material that is carried over from the production of any or all of the three Bacillus species, such as a dry milk product, and/or a carrier that does not materially affect the structure, function, novel and/or basic features of the three Bacillus species.

ASL Combination: Refers to a combination, or a composition, of DFMs including only Bacillus amyloliquefaciens, Bacillus subtilis and Bacillus licheniformis. In some disclosed embodiments, “ASL combination” refers to a composition for administration to a subject, particularly to an animal, and even more particularly to an avian, such as chickens and turkeys, that consists of or consists essentially of Bacillus amyloliquefaciens, Bacillus subtilis and Bacillus licheniformis. In other embodiments, “ASL combination” refers to Bacillus amyloliquefaciens, Bacillus subtilis and Bacillus licheniformis administered in combination without any other DFMs. A person of ordinary skill in the art will understand that the ASL combination may include additional residual material that is carried over from the production of any or all of the three Bacillus species, such as a dry milk product, and/or a carrier that does not materially affect the structure, function, novel and/or basic features of the three Bacillus species.

ASLC Combination: Refers to a combination, or a composition, of DFMs including only Bacillus amyloliquefaciens, Bacillus subtilis, Bacillus licheniformis and Bacillus coagulans. In some disclosed embodiments, “ASLC combination” refers to a composition for administration to a subject, particularly to an animal, and even more particularly to an avian, such as chickens and turkeys, that consists of or consists essentially of Bacillus amyloliquefaciens, Bacillus subtilis, Bacillus licheniformis and Bacillus coagulans. In other embodiments, “ASLC combination” refers to Bacillus amyloliquefaciens, Bacillus subtilis, Bacillus licheniformis and Bacillus coagulans administered in combination without any other DFMs. A person of ordinary skill in the art will understand that the ASLC combination may include additional residual material that is carried over from the production of any or all of the four Bacillus species, such as a dry milk product, and/or a carrier that does not materially affect the structure, function, novel and/or basic features of the four Bacillus species.

Direct fed microbial: A composition that contains live and/or viable microorganisms, typically bacteria and/or yeast, that provides a beneficial effect on an animal, such as, but not limited to, an antimicrobial effect including decreased bacterial counts, improved feed conversion rate, improved weight gain, improved health parameters, reduced mortality rate, and/or improved lesion scores.

Effective amount: A quantity or concentration of a specified compound, composition or combination sufficient to achieve an effect.

Essential Oil: An essential oil is a concentrated liquid extract obtained from a plant. As used herein, the term “essential oil” refers to a compound or mixture of compounds that has been removed from the plant, for example, by distillation and/or pressure. In some embodiments, an essential oil is a concentrated hydrophobic liquid containing volatile chemical compounds from the plant. The essential oil of the plant may include compounds that give the plant its characteristic scent and/or flavor. Exemplary essential oils include, but are not limited to, Peppermint, Lavender, Sandalwood, Bergamot, Rose, Chamomile, Ylang-Ylang, Tea Tree, Jasmine, Lemon, Cinnamon, lemongrass, Clary sage, Eucalyptus, Rosemary, Orange, Lime, Spearmint, Grapefruit, Frankincense, Oregano, and combinations thereof.

Feed conversion rate: A measure of the efficiency of an animal to convert feed mass into increased body mass. Typically, the feed conversion rate is calculated as pounds of feed divided by pounds of weight gain, and therefore may be expressed as a dimensionless number. The feed conversion rate is also known in the art as the feed conversion ratio, or feed efficiency.

Glucocorticoid: A class of steroid hormones that bind to the glucocorticoid receptors in vertebrate animal cells. Exemplary endogenous glucocorticoids include cortisol (hydrocortisone) and corticosterone.

Halo, halide or halogen: Fluoro, chloro, bromo or iodo.

Haloaliphatic: An aliphatic moiety substituted with one or more halogens, including haloalkyl, haloalkenyl, or haloalkynyl groups, or cyclic versions thereof. CF₃ is an exemplary haloaliphatic moiety.

Heteroaliphatic: An aliphatic compound or group having at least one heteroatom and at least one carbon atom, i.e., one or more carbon atoms from an aliphatic compound or group comprising at least two carbon atoms, has been replaced with an atom having at least one lone pair of electrons, typically nitrogen, oxygen, phosphorus, silicon, or sulfur. Heteroaliphatic compounds or groups may be substituted or unsubstituted, branched or unbranched, chiral or achiral, and/or acyclic or cyclic, such as a cycloheteroaliphatic group.

Heteroaryl: An aromatic group or moiety of, unless specified otherwise, from 5 to 15 ring atoms comprising at least one carbon atom and at least one heteroatom, such as N, S, O, P or Si. A heteroaryl group or moiety may comprise a single ring (e.g., pyridinyl) or multiple condensed rings (e.g., indolyl). Heteroaryl groups or moiety may be, for example, monocyclic, bicyclic, tricyclic or tetracyclic. Unless otherwise stated, a heteroaryl group or moiety may be substituted or unsubstituted.

Heterocyclyl: Aromatic and non-aromatic ring systems, and more specifically a stable three- to fifteen-membered ring moiety, comprising at least one carbon atom, and typically plural carbon atoms, and at least one, such as from one to five, heteroatoms. The heteroatom(s) may be nitrogen, phosphorus, oxygen, silicon or sulfur atom(s). The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and any nitrogen, phosphorus, carbon, silicon or sulfur atoms in the heterocyclyl moiety can be optionally oxidized to various oxidation states. For convenience, nitrogens, particularly but not exclusively, those defined as annular aromatic nitrogens, are meant to include their corresponding N-oxide form, although not explicitly defined as such in a particular example. Thus, for a compound having, for example, a pyridinyl ring, the corresponding pyridinyl-N-oxide is included as another compound of the invention, unless expressly excluded or excluded by context. In addition, annular nitrogen atoms can be optionally quaternized. Heterocycle includes heteroaryl moieties, and cycloheteroaliphatic moieties, which are heterocyclyl rings which are partially or fully saturated. Examples of heterocyclyl groups include, but are not limited to, tetrahydropyrolyl, piperidinyl, piperazinyl, pyridinyl, indolyl, and morpholinyl.

Ligand: An ion or molecule associated with, such as bound or bonded to, at least one metal atom or ion, such as a transition metal atom or a transition metal ion, to form a chelate. A ligand may bind to the metal or metal ion via coordinate bonding between the metal or metal ion and one or more negatively charged moieties on the ligand; one or more lone pairs of electrons on the ligand, such as from an oxygen, nitrogen, or sulfur atom; or a combination thereof. Ligands can be further characterized as monodentate, bidentate, tridentate, tetradentate, polydentate, etc., depending upon the number of donor atoms of the ion or molecule that bind to the central atom or ion. A ligand may be described as being derived from a chelator when the chemical structure of the ligand is different from the chemical structure of the chelator used to form the metal complex. For example, a ligand comprising a carboxylate moiety (CO₂⁻) is derived from the corresponding carboxylic acid (CO₂H) chelator by deprotonation of the carboxylic acid. Similarly, a ligand comprising an O⁻ moiety may be derived from a corresponding chelator comprising a hydroxyl (OH) moiety.

Mannans: A class of polysaccharides including the sugar mannose. The mannans family includes pure mannans (i.e., the polymer backbone comprises of mannose monomers), glucomannan (the polymer backbone comprises mannose and glucose), and galactomannan (mannans or glucomannan in which single galactose residues are linked to the polymer backbone). Mannans are found in cell walls of some plant species and yeasts, and may be provided as extracts of such plant species and/or yeasts.

Mineral clay: The term “mineral clay” refers to hydrous aluminum silicates. Mineral clays usually include minor amounts of impurities, such as potassium, sodium, calcium, magnesium, and/or iron.

Oligopeptides: A peptide comprising plural amino acids, such as between two and twenty amino acid residues. In some embodiments, oligopeptides can include, or be referred to herein as, dipeptides, tripeptides, tetrapeptides, pentapeptides, etc., or alternatively as dimers, trimers, tetramers, pentamers, etc., depending upon the number of amino acid residues that together form the peptide.

Peptide: A compound comprising two or more amino acid residues linked in a chain, where the carboxylic acid group of one amino acid is joined to the amino group of another amino acid by a peptide bond, such as —OC—NH—.

Pharmaceutically acceptable: The term “pharmaceutically acceptable” refers to a substance that can be taken into a subject without significant adverse toxicological effects on the subject, including a non-human animal subject.

Polyphenols: A class of natural, synthetic, or semisynthetic organic chemicals characterized by the presence of plural phenolic

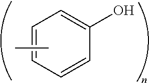

structural units.

Saponin: A class of chemical compounds, one of many secondary metabolites found in natural sources. Saponins are found in particular abundance in various plant species, such as quillaja and yucca. More specifically, saponins are amphipathic glycosides grouped, in terms of structure, by their composition. In certain embodiments, a saponin comprises one or more hydrophilic glycoside moieties combined with a lipophilic triterpene or a triterpene derivative, a steroid or a steroidal derivative, or both.

Strain: A strain refers to two members of the same species having a discernible phenotypic and/or genetic difference.

Subject: Any non-ruminant animal or human, and most typically “subject” refers herein to avians, including poultry, such as a chicken, turkey, goose, duck, Cornish game hen, quail, partridge, pheasant, guinea-fowl, ostrich, emu, swan, or pigeon, particularly a chicken or turkey.

Substituted: When used to modify a specified group or moiety, the term ‘substituted’ means that at least one, and perhaps two or more, typically, 1, 2, 3, or 4, hydrogen atoms of the specified group or moiety is independently replaced with the same or different substituent groups as defined herein, unless the context indicates otherwise or a particular structural formula precludes substitution. In a particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” substituent may be unsubstituted or substituted, but an “unsubstituted alkyl” may not be substituted.

Unless otherwise specified herein, exemplary substituent groups include, but are not limited to, aliphatic, such as alkyl; haloalkyl, such as —CF₃; —N(R′)₂; aromatic; heteroaliphatic; halo; —OR′; —SR′; —CH₂OR′; —(C(R′)₂)ₘ—C(O)—R′, where m is from 0 to 4; —CN; —Si(R′)₃; —Si(OR′)₃; or combinations thereof; wherein each R′ independently is H, or aliphatic, such as alkyl.

Additionally, in embodiments where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(aryl-1)-(aryl-2)-(aryl-3), aryl-3 can only be substituted with substituents that are not themselves substituted.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

Effective amount: A quantity or concentration of a specified compound, composition or combination sufficient to achieve an effect in a subject.

Vitamin: Includes Vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin, and derivative and analogs thereof.

Additional disclosure is provided by U.S. patent application Ser. No. 14/699,740, U.S. patent application Ser. No. 13/566,433, U.S. patent application Ser. No. 13/872,935, U.S. Patent Publication No. 2013/0017211, U.S. Patent Publication No. 2012/0156248, U.S. Patent Publication No. 2007/0253983, U.S. Patent Publication No. 2007/0202092, U.S. Patent Publication No. 2007/0238120, U.S. Patent Publication No. 2006/0239992, U.S. Patent Publication No. 2005/0220846, U.S. Patent Publication No. 2005/0180964, and Australian Patent Application No. 2011/201420, each of which is incorporated herein by reference in its entirety.

### II. Combination and/or Composition Comprising a Bacillus Combination and an Essential Oil

Disclosed herein are embodiments of a combination and/or composition comprising a Bacillus combination and an essential oil composition. In some embodiments, the combination and/or composition may further comprise yucca or an extract thereof, quillaja or an extract thereof, or a combination of yucca and quillaja and/or extracts thereof.

Certain aspects of the present disclosure concern the discovery that administering a combination and/or composition comprising a Bacillus combination and an essential oil composition to a subject provides a substantial benefit to the subject compared to a subject that is not administered the combination and/or composition. Further aspects of the present disclosure concern the discovery that administering a combination and/or composition comprising a Bacillus combination, an essential oil composition, and yucca, quillaja, or extracts thereof, to a subject provides a substantial benefit to the subject compared to a subject that is not administered the combination and/or composition.

The combination and/or composition may be administered as a composition or as a combination. With particular reference to poultry, a combination and/or composition comprising a Bacillus combination, an essential oil composition, and optionally, yucca and/or quillaja, or extracts thereof, provides a substantial benefit with respect to one or more of feed conversion rate, average body weight, average body weight gain, body weight coefficient of variation, bird mortality, lesion scores, bacterial incidence including Salmonella/E. Coli/Clostridium perfringens (CP) incidence, and/or oocysts in fecal matter relative to poultry that are not fed the combination and/or composition.

A. Bacillus Combination

A Bacillus combination as disclosed herein is a combination or composition comprising three or four DFMs selected from Bacillus coagulans, Bacillus subtilis, Bacillus licheniformis and Bacillus amyloliquefaciens. A CSL combination is a combination or composition comprising the DFMs Bacillus coagulans, Bacillus subtilis and Bacillus licheniformis and no additional DFMs. An ASL combination is a combination or composition comprising the DFMs Bacillus amyloliquefaciens, Bacillus subtilis and Bacillus licheniformis. In some embodiments, an ASL combination comprises, consists essentially of, or consists of Bacillus amyloliquefaciens, Bacillus subtilis and Bacillus licheniformis and no additional DFMs. An ASLC combination is a combination or composition comprising the DFMs Bacillus amyloliquefaciens, Bacillus subtilis, Bacillus licheniformis and Bacillus coagulans. In some embodiments, an ASLC combination comprises, consists essentially of, or consists of Bacillus amyloliquefaciens, Bacillus subtilis, Bacillus licheniformis and Bacillus coagulans, but no other additional DFMs. With respect to the Bacillus combination, the term “consisting essentially of” means that the Bacillus combination does not include any additional DFM, such as no additional Bacillus species, but may include additional components suitable for preparing a Bacillus combination, such as a vehicle, carrier, filler, preservative, stabilizer, or any combination thereof. In some embodiments, the Bacillus combination comprises a limestone carrier.

In some embodiments, one or more of the bacillus in the Bacillus combination is provided as a bacillus spore, and in certain embodiments, all of the bacillus in the Bacillus combination are provided as spores.

In some embodiments, one or more of the bacillus in the Bacillus combination is dehydrated, such as by freeze drying or lyophilization, spray drying or other suitable dehydration techniques. Dehydration, such as by freeze drying or spray drying may improve the stability and/or shelf-life of the bacteria. In certain embodiments, all the bacillus in the Bacillus combination are freeze dried.

i) Bacillus Strains

A person of ordinary skill in the art will appreciate that any strain, or combinations of strains, of Bacillus coagulans, Bacillus subtilis, Bacillus licheniformis and/or Bacillus amyloliquefaciens can be used in the Bacillus combination. As used herein the terms “Bacillus amyloliquefaciens,” “Bacillus coagulans,” “Bacillus subtilis” and “Bacillus licheniformis” independently may refer to a single strain of the respective Bacillus species, or to multiple strains, such as 2, 3, 4, 5, 6, 7, 8, 9, 10 or more strains, of each respective Bacillus species. Solely by way of example and without limitation, certain acceptable exemplary strains of each Bacillus species are listed below. In certain embodiments, a Bacillus combination includes one or more of Bacillus amyloliquefaciens TOA5001, Bacillus coagulans GBI-30 strain, ATCC Designation Number PTA-6086, Bacillus licheniformis OBT618, and Bacillus subtilis strain OBT 1224, and in a particular embodiments, the Bacillus combination includes Bacillus amyloliquefaciens TOA5001, Bacillus coagulans GBI-30 strain, ATCC Designation Number PTA-6086, Bacillus licheniformis OBT618, and Bacillus subtilis strain OBT 1224.

### Bacillus coagulans Strains

Bacillus coagulans Hammer ATCC® BAA-738™ strain LMG 17453, Logan B0934, NCTC 3992, Vitek #202384, Bacillus coagulans Hammer ATCC® 7050™ strain NRS 609, NCIB 9365, NCTC 10334, DSM 1, CCM 2013, WDCM 00002, Bacillus coagulans Hammer ATCC® 8038™ strain NCA 43P, NCIB 8080, NRS 770, DSM 2312 deposited with ATCC as Bacillus thermoacidurans by Berry, Bacillus coagulans Hammer ATCC® 10545™ strain NRS 784, NCIB 8041, DSM 2311, CCM 1082, deposited with ATCC as Bacillus dextrolacticus by Andersen and Werkman, Bacillus coagulans Hammer ATCC® 11014™ strain NRS T27, 78G, DSM 2383, Bacillus coagulans Hammer ATCC® 11369™ strain C, DSM 2384 deposited with ATCC as Bacillus dextrolacticus by Andersen and Werkman, Bacillus coagulans Hammer ATCC® 12245™ strain NCA 308, DSM 2308, NCIB 8870, Bacillus coagulans Hammer ATCC® 15949™ strain NCA 4259, DSM 2385, Bacillus coagulans Hammer ATCC® 23498™ strain M-39, DSM 2314, NCIB 10276 deposited with ATCC as Bacillus racemilacticus by Nakayama and Yanoshi, Bacillus coagulans Hammer ATCC® 31284™ deposited with ATCC as Lactobacillus sporogenes by Horowitz-Wiassowa and Nowotelnow, Ganeden Biotech Inc.'s Bacillus coagulans GBI-30 strain, ATCC Designation Number PTA-6086, Bacillus coagulans Hammer ATCC® 53595™ strain PM-1000, Bacillus coagulans Hammer strain DSM 2350, NRRL-NRS 2012, Bacillus coagulans Hammer strain DSM 2356, NCIB 8523, N.R.Smith (NRS) 798, B. Hammer Iowa State College 200, Bacillus coagulans Hammer strain DSM 30760, Bacillus coagulans Hammer strain STI09070 (IMET), 1032-005, Bacillus coagulans Hammer strain STI09076 (IMET), 1141-003, Bacillus coagulans Hammer strain STI09080 (IMET), 1136-014, Bacillus coagulans Hammer strain STI09208 (IMET), 491-25, Bacillus coagulans Hammer strain STI09210 (IMET), 485-59, Bacillus coagulans Hammer strain NCIB 700460, Th1, Bacillus coagulans Hammer strain NCIB 701099, BG5, TH27 (205), Bacillus coagulans Hammer strain NCIB 701159, 254, and Bacillus coagulans Hammer strain NCIB 701164, 259.

### Bacillus licheniformis Strains

Bacillus licheniformis (Weigmann) Chester ATCC® 6598™ strain NRS 745 deposited with ATCC as Bacillus subtilis by (Ehrenberg) Cohn, Bacillus licheniformis (Weigmann) Chester ATCC® 6634™ strain NRS 304, Bacillus licheniformis (Weigmann) Chester ATCC® 8480™ strain NRS 1128, Bacillus licheniformis (Weigmann) Chester ATCC® 9259™, Bacillus licheniformis (Weigmann) Chester ATCC® 9789™ strain AMNH 723, ATCC 102, ATCC 4527, ATCC 8243, ATCC 9800, NCTC 2586, NCTC 6346, NRS 243, NRS 978, W. Ford 1, DSM 8785, DSM 46308, BU 171, CCDB b-30, CCEB 631, CCM 2205, CN 1060, HNCMB 101012, IFO 12195, IFO 12196, IMET 11025, NBRC 12195, NBRC 12196, NCDO 735, NCDO 835, NCIB 6346, NCIB 8059, NCIB 8061, OUT 8367, OUT 8368, Smith 243, Smith 978, HankeyB13 deposited with ATCC as Bacillus subtilis by (Ehrenberg) Cohn, Bacillus licheniformis (Weigmann) Chester ATCC® 9945™ strain NRS 712, NCIB 8062 deposited with ATCC as Bacillus subtilis by (Ehrenberg) Cohn, Bacillus licheniformis (Weigmann) Chester ATCC® 9945a™ strain CD-2, NCIB 11709, Bacillus licheniformis (Weigmann) Chester ATCC® 10716™ strain ATCC 11944, BS 2181, Boots 1343, CCM 2181, FDA BT1, NCIB 8874, NRS 1330, Tracy I, DSM 603, IFO 12199, NBRC 12199, Bacillus licheniformis (Weigmann) Chester ATCC® 11945™ strain 1331, FDA BT3, Bacillus licheniformis (Weigmann) Chester ATCC® 11946™ strain 1333, B-1001, Bacillus licheniformis (Weigmann) Chester ATCC® 12139™ strain CSC deposited with ATCC as Bacillus subtilis by (Ehrenberg) Cohn, Bacillus licheniformis (Weigmann) Chester ATCC® 12713™ strain PRL B479, NRRL B-1001, Bacillus licheniformis (Weigmann) Chester ATCC® 12759™ strain ATCC 11560, Damodaron P-8, LMG 7560, NRS 1415, Vitek #200148, NCIB 8549, HankeyB133, P8, Bacillus licheniformis (Weigmann) Chester ATCC® 12759-MINI-PACK™ strain ATCC 11560, Damodaron P-8, LMG 7560, NRS 1415, Vitek #200148, Bacillus licheniformis (Weigmann) Chester ATCC® 13438™ Strain NCTC 8233, M. II strain, Bacillus licheniformis (Weigmann) Chester ATCC® 14409™ strain 620, NRS 1114, NCIB 1042, deposited with ATCC as Bacillus abysseus by ZoBell and Upham, Bacillus licheniformis (Weigmann) Chester ATCC® 14580™ strain (Gibson) 46, NCIB 9375, NCTC 10341, NRS 1264, DSM 13, CCM 2145, IFO 12200, NBRC 12200, WDCM 00068, Bacillus licheniformis (Weigmann) Chester ATCC® 14580D-5™ strain designation: Genomic DNA from Bacillus licheniformis Strain 46 [ATCC® 14580™], Bacillus licheniformis (Weigmann) Chester ATCC® 14594™, Bacillus licheniformis (Weigmann) Chester ATCC® 21038™ strain L-065, Bacillus licheniformis (Weigmann) Chester ATCC® 21039™, Bacillus licheniformis (Weigmann) Chester ATCC® 21415™ strain NS 1 deposited with ATCC as Bacillus subtilis by (Ehrenberg) Cohn, Bacillus licheniformis (Weigmann) Chester ATCC® 21417™ strain M deposited with ATCC as Bacillus subtilis (Ehrenberg) Cohn, Bacillus licheniformis (Weigmann) Chester ATCC® 21418™ deposited with ATCC as Bacillus subtilis by (Ehrenberg) Cohn, Bacillus licheniformis (Weigmann) Chester ATCC® 21424™ strain DSM 1969, Bacillus licheniformis (Weigmann) Chester ATCC® 21610™ strain B-201-7 deposited with ATCC as Bacillus subtilis (Ehrenberg) Cohn, Bacillus licheniformis (Weigmann) Chester ATCC® 21667™ strain FD 23612, Bacillus licheniformis (Weigmann) Chester ATCC® 21733™ strain DSM 1913 deposited with ATCC as Bacillus subtilis by (Ehrenberg) Cohn, Bacillus licheniformis (Weigmann) Chester ATCC® 25972™ strain 749/C, DSM 8782, DSM 46217, IMET10723, NCIB 9443, Bacillus licheniformis (Weigmann) Chester ATCC® 27326™ strain OM-81, Bacillus licheniformis (Weigmann) Chester ATCC® 27811™ strain 584, FERM-P 1038, Bacillus licheniformis (Weigmann) Chester ATCC® 31667™ strain DG 14, Bacillus licheniformis (Weigmann) Chester ATCC® 31972™ strain PM-3, Bacillus licheniformis (Weigmann) Chester ATCC® 33632™ strain (IOC) 2390, NCIB 11672, Bacillus licheniformis (Weigmann) Chester ATCC® 39326™, Bacillus licheniformis (Weigmann) Chester ATCC® 53757™ strain PWD-1, Bacillus licheniformis (Weigmann) Chester ATCC® 53926™ strain E312, Bacillus licheniformis (Weigmann) Chester ATCC® 55768™ strain O.W.U. 138B [OWU 138B], Bacillus licheniformis (Weigmann) Chester strain DSM 15, C, Bacillus licheniformis (Weigmann) Chester strain DSM 392, Bacillus licheniformis (Weigmann) Chester strain DSM 394, Bacillus licheniformis (Weigmann) Chester strain DSM 7259, NRRL-NRS 1263, Bacillus licheniformis (Weigmann) Chester strain DSM 7459, Bacillus licheniformis (Weigmann) Chester strain DSM 11258, Bacillus licheniformis (Weigmann) Chester strain DSM 11259, Bacillus licheniformis (Weigmann) Chester strain DSM 12369, Bacillus licheniformis (Weigmann) Chester strain DSM 12370, Bacillus licheniformis (Weigmann) Chester strain DSM 26543, Bacillus licheniformis (Weigmann) Chester strain DSM 28096, Bacillus licheniformis (Weigmann) Chester strain DSM 28591, Bacillus licheniformis (Weigmann) Chester strain DSM 30523, Bacillus licheniformis (Weigmann) Chester strain DSM 30535, Bacillus licheniformis (Weigmann) Chester strain DSM 30542, Bacillus licheniformis (Weigmann) Chester strain DSM 30585, Bacillus licheniformis (Weigmann) Chester strain DSM 30615, Bacillus licheniformis (Weigmann) Chester strain DSM 30620, Bacillus licheniformis (Weigmann) Chester strain DSM 30624, Bacillus licheniformis (Weigmann) Chester strain DSM 30643, Bacillus licheniformis (Weigmann) Chester strain DSM 30654, Bacillus licheniformis (Weigmann) Chester strain DSM 30724, Bacillus licheniformis (Weigmann) Chester strain DSM 30766, Bacillus licheniformis (Weigmann) Chester strain DSM 30769, Bacillus licheniformis (Weigmann) Chester strain DSM 30778, Bacillus licheniformis (Weigmann) Chester strain DSM 30779, Bacillus licheniformis (Weigmann) Chester strain DSM 30865, Bacillus licheniformis (Weigmann) Chester strain DSM 30926, Bacillus licheniformis (Weigmann) Chester strain DSM 30959, Bacillus licheniformis (Weigmann) Chester strain DSM 30960, Bacillus licheniformis (Weigmann) Chester strain DSM 30961, Bacillus licheniformis (Weigmann) Chester strain DSM 30976, Bacillus licheniformis (Weigmann) Chester strain DSM 31019, Bacillus licheniformis (Weigmann) Chester strain DSM 100653, Bacillus licheniformis (Weigmann) Chester strain DSM 100655, Bacillus licheniformis (Weigmann) Chester strain DSM 103059, Bacillus licheniformis (Weigmann) Chester strain NCIB 1525, 1229, Bacillus licheniformis (Weigmann) Chester strain NCIB 6816, Glaxo 417, Bacillus licheniformis (Weigmann) Chester strain NCIB 7224, Loos, Bacillus licheniformis (Weigmann) Chester strain NCIB 8536, P1, Bacillus licheniformis (Weigmann) Chester strain NCIB 8537, Ho, Bacillus licheniformis (Weigmann) Chester strain NCIB 9536, Gibson 1319, NRS 1553, Bacillus licheniformis (Weigmann) Chester strain NCIB 9667, 1, Bacillus licheniformis (Weigmann) Chester strain NCIB 9668, 2, Bacillus licheniformis (Weigmann) Chester strain NCIB 9669, 3, Bacillus licheniformis (Weigmann) Chester strain NCIB 10689, Bacillus licheniformis (Weigmann) Chester strain NCIB 11143, Bacillus licheniformis (Weigmann) Chester strain NCIB 11643, YNS7712R, Bacillus licheniformis (Weigmann) Chester strain NCIB 13497, Bacillus licheniformis (Weigmann) Chester strain NCIB 14014, DA33, Bacillus licheniformis B1 (NRRL Deposit Number B-50907), Bacillus subtilis B2 (Deposit Number B-50908), Bacillus licheniformis RW25 (NRRL Deposit Number B-50911), Bacillus licheniformis RW32 (NRRL Deposit Number B-50912), and Bacillus licheniformis RW41 (NRRL Deposit Number B-50913), Bacillus licheniformis BL21 (NRRL B-50134), Bacillus licheniformis 3-12a (NRRL B-50504), Bacillus licheniformis 4-2a (NRRL B-50506), Bacillus licheniformis 842 (NRRL B-50516), Bacillus licheniformis DSM 5749 (BioPlus® 2B, Chr. Hansen Bio Systems), and Bacillus licheniformis OBT618 (ATCC PTA-122188, Osprey Biotechnics).

### Bacillus subtilis Strains

Bacillus subtilis (Ehrenberg) Cohn ATCC® 82™ strain AMC, ATCC 8037, NRS 315, Bacillus subtilis (Ehrenberg) Cohn ATCC® 82D-5™ strain designation: Genomic DNA from Bacillus subtilis strain AMC [ATCC® 82™], Bacillus subtilis (Ehrenberg) Cohn ATCC® 465™ strain NRS 743, Bacillus subtilis (Ehrenberg) Cohn ATCC® 4529™ strain 3, ATCC 8013, NCTC 2588, NRS 1004 deposited with ATCC as Bacillus vulgatus by Trevisan, Bacillus subtilis (Ehrenberg) Cohn ATCC® 4925™ strain NRS 740 deposited with ATCC as Bacillus nigrificans by Fabian and Nienhuis, Bacillus subtilis (Ehrenberg) Cohn ATCC® 4944™ strain NCTC, NRS 1106 deposited with ATCC as Bacillus parvus, Bacillus subtilis subspecies subtilis (Ehrenberg) Cohn ATCC® 6051™ strain Marburg strain, ATCC 6051-U, CCM 2216, CCRC 10255, CCUG 163B, CFBP 4228, CIP 52.65, DSM 10, IAM 12118, IFO 12210, IFO 13719, IFO 16412, IMET 10758, JCM 1465, LMG 7135, NCAIM B.01095, NCCB 32009, NCCB 53016, NCCB 70064, NCFB 1769, NCIB 3610, NCTC 3610, NRRL B-4219, NRS 1315, NRS 744, VKM B-501, NBRC 13719 deposited with ATCC as Bacillus subtilis (Ehrenberg) Cohn, Bacillus subtilis (Ehrenberg) Cohn ATCC® 6051a™ strain P31K6, Bacillus subtilis bacteriophage phi-e ATCC® 6051-B1™ strain Phi-e deposited with ATCC as phi e, Bacillus subtilis (Ehrenberg) Cohn ATCC® 6460™ strain NRS 259 deposited with ATCC as Bacillus aterrimus by Lehmann and Neumann, Bacillus subtilis (Ehrenberg) Cohn ATCC® 6461™ strain NRS 275, CN 2192, NCIB 8055 deposited with ATCC as Bacillus aterrimus by Lehmann and Neumann, Bacillus subtilis subspecies spizizenii Nakamura et al. ATCC® 6633™ strain NRS 231, DSM 347, CCM 1999, IAM 1069, NCIB 8054, NCTC 10400, WDCM 00003 deposited with ATCC as Bacillus subtilis (Ehrenberg) Cohn, Bacillus subtilis subspecies spizizenii Nakamura et al. ATCC® 6633D-5™ strain designation: Genomic DNA from Bacillus subtilis subspecies spizizenii strain NRS 231 [ATCC® 6633™] deposited with ATCC as Bacillus subtilis (Ehrenberg) Cohn, Bacillus subtilis subspecies spizizenii Nakamura et al. ATCC® CRM-6633™ strain NRS 231 deposited with ATCC as Bacillus subtilis (Ehrenberg) Cohn, Bacillus subtilis subspecies spizizenii Nakamura et al. ATCC® 6633-MINI-PACK™ strain NRS 231 deposited with ATCC as Bacillus subtilis (Ehrenberg) Cohn, Bacillus subtilis (Ehrenberg) Cohn ATCC® 6984™ strain NRS 747 deposited with ATCC as Bacillus vulgatus subspecies hydrolyticus, Bacillus subtilis (Ehrenberg) Cohn ATCC® 7003™ strain NRS 730, Bacillus subtilis (Ehrenberg) Cohn ATCC® 7058™ strain NRS 351, Bacillus subtilis (Ehrenberg) Cohn ATCC® 7059™ strain NRS 352, Bacillus subtilis (Ehrenberg) Cohn ATCC® 7060™ strain NRS 659, Bacillus subtilis (Ehrenberg) Cohn ATCC® 7067™ strain NRS 238, ATCC 7974, ATCC 8012, Bacillus subtilis (Ehrenberg) Cohn ATCC® 7480™ strain NRS 1107 deposited with ATCC as Bacillus endoparasiticus by (Benedek) Benedek, Bacillus subtilis (Ehrenberg) Cohn ATCC® 8188™ strain ATCC 8450, NRS 773 deposited with ATCC as Tyrothrix minimus, Bacillus subtilis (Ehrenberg) Cohn ATCC® 8473™ strain NRS 762, Bacillus subtilis (Ehrenberg) Cohn ATCC® 9466™ strain designation: FDA strain PCI 220 [BUCSAV 170, NCIB 8159, NRRL B-558, NRS 1088], Bacillus subtilis (Ehrenberg) Cohn ATCC® 9524™ strain 3R9675, NRS 1109, Bacillus subtilis (Ehrenberg) Cohn ATCC® 9799™ strain NCTC 6276, NRS 1125, Bacillus subtilis (Ehrenberg) Cohn ATCC® 9858™ strain NRS 237, NCIB 8063, Bacillus subtilis (Ehrenberg) Cohn ATCC® 9943™ strain NRS 979, Bacillus subtilis (Ehrenberg) Cohn ATCC® 10774™ strain BU169, NCIB 8872, Bacillus subtilis (Ehrenberg) Cohn ATCC® 10783™ strain NRRL B-543, Bacillus subtilis (Ehrenberg) Cohn ATCC® 11774™ strain NCTC 8236, DSM 2109, Bacillus subtilis (Ehrenberg) Cohn ATCC® 11838™ strain AMC 46-A-6 (strain I), NCIB 8850, Bacillus subtilis (Ehrenberg) Cohn ATCC® 12100™ strain NCA 1558, ND 957, Bacillus subtilis (Ehrenberg) Cohn ATCC® 12432™ strain MB 32, 56R188, ATCC 13597, NCIB 8993, Bacillus subtilis (Ehrenberg) Cohn ATCC® 12695™ strain 51-52, Bacillus subtilis (Ehrenberg) Cohn ATCC® 12711™ strain PRL B92, Ra, Bacillus subtilis (Ehrenberg) Cohn ATCC® 13542™, Bacillus subtilis (Ehrenberg) Cohn ATCC® 13933™ strain NRRL B-1471, Bacillus subtilis (Ehrenberg) Cohn ATCC® 13952™ strain 1346, Bacillus subtilis (Ehrenberg) Cohn ATCC® 14410™ strain 625, NRS 1115 deposited with ATCC as Bacillus borborokoites by ZoBell and Upham, Bacillus subtilis (Ehrenberg) Cohn ATCC® 14415™ strain 569, NRS 1120 deposited with ATCC as Bacillus submarinus by ZoBell and Upham, Bacillus subtilis (Ehrenberg) Cohn ATCC® 14416™ strain 576, NRS 1121 deposited with ATCC as Bacillus thalassokoites by ZoBell and Upham, Bacillus subtilis (Ehrenberg) Cohn ATCC® 14593™ strain IAM 1145, Bacillus subtilis (Ehrenberg) Cohn ATCC® 14617™ strain A-1625, Bacillus subtilis (Ehrenberg) Cohn ATCC® 14660™ strain C₃₀-1, Bacillus subtilis (Ehrenberg) Cohn ATCC® 14662™ strain C₃₀-109, Bacillus subtilis (Ehrenberg) Cohn ATCC® 14807™ strain MB-155, Bacillus subtilis (Ehrenberg) Cohn ATCC® 15040™ strain SX-67, Bacillus subtilis (Ehrenberg) Cohn ATCC® 15041™ strain SX-92, Bacillus subtilis (Ehrenberg) Cohn ATCC® 15134™ deposited with ATCC as Bacillus uniflagellatus by Mann, Bacillus subtilis (Ehrenberg) Cohn ATCC® 15183™ strain 309, Bacillus subtilis (Ehrenberg) Cohn ATCC® 15244™ strain 3369, Bacillus subtilis (Ehrenberg) Cohn ATCC® 15245™ strain 3349, IAM 1-3 deposited with ATCC as Bacillus natto by Sawamura, Bacillus subtilis (Ehrenberg) Cohn ATCC® 15476™ strain M-4-45, Bacillus subtilis (Ehrenberg) Cohn ATCC® 15477™ strain M-24-1 deposited with ATCC as Bacillus pumilus by Meyer and Gottheil, Bacillus subtilis (Ehrenberg) Cohn ATCC® 15561™ strain K-X-1, A-1, Bacillus subtilis (Ehrenberg) Cohn ATCC® 15563™ strain Marburg, Bacillus subtilis bacteriophage SP8 ATCC® 15563-B1™ strain SP8 deposited with ATCC as SP8 bacteriophage, Bacillus subtilis (Ehrenberg) Cohn ATCC® 15575™ strain SB 19, Bacillus subtilis (Ehrenberg) Cohn ATCC® 15811™ strain 5380, Bacillus subtilis (Ehrenberg) Cohn ATCC® 15818™ strain RIA 445, Bacillus subtilis (Ehrenberg) Cohn ATCC® 15819™ strain RIA 447, Bacillus subtilis (Ehrenberg) Cohn ATCC® 15841™, Bacillus subtilis bacteriophage S-a ATCC® 15841-B1™ strain S-a deposited with ATCC as S-a bacteriophage, Bacillus subtilis (Ehrenberg) Cohn ATCC® 19659™ strain PRD 66, IFO 13722, Bacillus subtilis (Ehrenberg) Cohn ATCC® 19659-MINI-PACK™ strain PRD 66, IFO 13722, Bacillus subtilis (Ehrenberg) Cohn ATCC® 21008™ strain 182-H-86 deposited with ATCC as Bacillus pumilus by Meyer and Gottheil, Bacillus subtilis (Ehrenberg) Cohn ATCC® 21183™ strain 5221, Bacillus subtilis (Ehrenberg) Cohn ATCC® 21228™ strain SC 8548, SO-4, DSM 1970, Bacillus subtilis (Ehrenberg) Cohn ATCC® 21331™ strain IFO 35, Bacillus subtilis (Ehrenberg) Cohn ATCC® 21332™ strain IAM 1213, Bacillus subtilis (Ehrenberg) Cohn ATCC® 21394™ strain 4-3-Ky, DSM 1971 deposited with ATCC as Bacillus subtilis subspecies sakainensis, Bacillus subtilis (Ehrenberg) Cohn ATCC® 21555™ strain Y 13, Bacillus subtilis (Ehrenberg) Cohn ATCC® 21556™ Bacillus subtilis (Ehrenberg) Cohn ATCC® 21742™ strain AHr-5, Bacillus subtilis (Ehrenberg) Cohn ATCC® 21770™ strain SP-3 deposited with ATCC as Bacillus cereus by Frankland and Frankland, Bacillus subtilis (Ehrenberg) Cohn ATCC® 21951™ strain 716, IFO 13322 deposited with ATCC as Bacillus pumilus by Meyer and Gottheil, Bacillus subtilis (Ehrenberg) Cohn ATCC® 23059™ strain W23, Bacillus subtilis (Ehrenberg) Cohn ATCC® 23856™ strain EMG 50, SB19, Bacillus subtilis (Ehrenberg) Cohn ATCC® 23857™ strain 168, Bacillus subtilis (Ehrenberg) Cohn ATCC® 23857D-5™ strain Designation: Genomic DNA from Bacillus subtilis strain 168 [ATCC® 23857™], Bacillus subtilis (Ehrenberg) Cohn ATCC® 23858™ strain EMG 52, Bacillus subtilis (Ehrenberg) Cohn ATCC® 23859™ strain EMG 53, Bacillus subtilis (Ehrenberg) Cohn ATCC® 25369™ strain 24028 deposited with ATCC as Bacillus pulvifaciens by Nakamura, Bacillus subtilis (Ehrenberg) Cohn ATCC® 27328™ strain C, Bacillus subtilis (Ehrenberg) Cohn ATCC® 27370™ strain 168 M, Bacillus subtilis bacteriophage SPO1 ATCC® 27370-B1™ strain SPO1 deposited with ATCC as SPO1, Bacillus subtilis (Ehrenberg) Cohn ATCC® 27505™ strain K49, HER 1346 deposited with ATCC as Bacillus subtilis subspecies amyloliquefaciens, Bacillus subtilis (Ehrenberg) Cohn ATCC® 27689™ strain SB168 (trp-), Bacillus subtilis (Ehrenberg) Cohn ATCC® 29056™ strain SB100, Bacillus subtilis (Ehrenberg) Cohn ATCC® 29233™ strain X6, Bacillus subtilis (Ehrenberg) Cohn ATCC® 31002™ strain Ahr.AUr-9, FERM-1998, Bacillus subtilis (Ehrenberg) Cohn ATCC® 31028™ strain FD 6404 deposited with ATCC as Bacillus globigii by Migula, Bacillus subtilis (Ehrenberg) Cohn ATCC® 31091™ strain 1054, IFO 13586, Bacillus subtilis (Ehrenberg) Cohn ATCC® 31094™ strain 1097, IFO 13621, Bacillus subtilis (Ehrenberg) Cohn ATCC® 31098™ strain 1027, IFO 13585 deposited with ATCC as Bacillus pumilus by Meyer and Gottheil, Bacillus subtilis subspecies subtilis (Ehrenberg) Nakamura et al. ATCC® 31578™ strain DSM 6223, RUB 331, Bacillus subtilis (Ehrenberg) Cohn ATCC® 31954™ strain MO7S-16/11, Bacillus subtilis (Ehrenberg) Cohn ATCC® 33234™ strain NCIB 10106, Bacillus subtilis (Ehrenberg) Cohn ATCC® 35021™ strain 5230, NRS 6, Bacillus subtilis (Ehrenberg) Cohn ATCC® 35854™ strain NRRL B-3411, Bacillus subtilis (Ehrenberg) Cohn ATCC® 35946™ strain OSU 75, Bacillus subtilis subspecies subtilis (Ehrenberg) Nakamura et al. ATCC® 37014™ strain DSM 6224, BD170, pSA2100, Bacillus subtilis subspecies subtilis (Ehrenberg) Nakamura et al. ATCC® 37015™ strain DSM 4514, BD170, NCIB 11624, pUB110, Bacillus subtilis subspecies subtilis (Ehrenberg) Nakamura et al. ATCC® 37108™ strain DSM 4873, BGSC 1E32, BR151, pPL608, Bacillus subtilis subspecies subtilis (Ehrenberg) Nakamura et al. ATCC® 37128™ strain DSM 4554, BGSC 1E18, pE194, Bacillus subtilis subspecies subtilis (Ehrenberg) Nakamura et al. ATCC® 39090™ strain DSM 6198, BGSC 1S53, Bacillus subtilis (Ehrenberg) Cohn ATCC® 39320™ strain MB 4488, Bacillus subtilis (Ehrenberg) Cohn ATCC® 39374™ strain MB 3575, Bacillus subtilis (Ehrenberg) Cohn ATCC® 39706™ strain B1-20, Bacillus subtilis (Ehrenberg) Cohn ATCC® 43223™ strain ABM261, Bacillus subtilis (Ehrenberg) Cohn ATCC® 49343™ strain IMVS 0101, Bacillus subtilis (Ehrenberg) Cohn ATCC® 49760™ deposited with ATCC as Bacillus globigii by Migula, Bacillus subtilis (Ehrenberg) Cohn ATCC® 49822™ deposited with ATCC as Bacillus globigii by Migula, Bacillus subtilis (Ehrenberg) Cohn ATCC® 55033™ strain SMS274, Bacillus subtilis (Ehrenberg) Cohn ATCC® 55060™ strain MB 4974, Bacillus subtilis (Ehrenberg) Cohn ATCC® 55405™ strain 300, Bacillus subtilis subspecies inaquosorum ATCC® 55406™ strain DA33 deposited with ATCC as Bacillus licheniformis (Weigmann) Chester, Bacillus subtilis (Ehrenberg) Cohn ATCC® 55422™ strain SC 15257, Bacillus subtilis (Ehrenberg) Cohn ATCC® 55614™ strain 1.2, AQ153, Bacillus subtilis (Ehrenberg) Cohn ATCC® 55675™ strain BPO1, Bacillus subtilis subspecies subtilis (Ehrenberg) Nakamura et al. strain DSM 402, BRC 111470, NCIB 10106, Bacillus subtilis subspecies spizizenii Nakamura et al. strain DSM 618, Bacillus subtilis subspecies spizizenii Nakamura et al. strain DSM 1087, Bacillus subtilis (Ehrenberg) Cohn strain DSM 1088, IFO 13169, NBRC 13169, OUT 8353, Bacillus subtilis (Ehrenberg) Cohn strain DSM 1089, IFO 3026, NBRC 3026, OUT 8350, Bacillus subtilis subspecies subtilis (Ehrenberg) Nakamura et al. strain DSM 1090, OUT 8424, Bacillus subtilis subspecies subtilis (Ehrenberg) Nakamura et al. strain DSM 1091, OUT 8425, Bacillus subtilis (Ehrenberg) Cohn strain DSM 1092, IFO 3009, NBRC 3009, OUT 8235, Bacillus subtilis subspecies subtilis (Ehrenberg) Nakamura et al. strain DSM 3256, IAM 1213, Bacillus subtilis subspecies subtilis (Ehrenberg) Nakamura et al. strain DSM 3257, IAM 1259, Bacillus subtilis (Ehrenberg) Cohn strain DSM 3258, IAM 1260, Bacillus subtilis (Ehrenberg) Cohn strain DSM 4181, NCA 72-52, SA 22, Bacillus subtilis subspecies subtilis (Ehrenberg) Nakamura et al. strain DSM 4393, pC194, SB202, Bacillus subtilis (Ehrenberg) Cohn strain DSM 4449, natto 3335 UM4, Bacillus subtilis (Ehrenberg) Cohn strain DSM 4450, natto 3335 UM8, pLS20, pBC16, Bacillus subtilis (Ehrenberg) Cohn strain DSM 4451 Bacillus subtilis (Ehrenberg) Cohn strain DSM 4515, DB163, pGR71, Bacillus subtilis (Ehrenberg) Cohn strain DSM 4608, BR157, pMW1, Bacillus subtilis subspecies subtilis (Ehrenberg) Nakamura et al. strain DSM 4750, 1E7, BGSC 1E7, pE194-cop6, Bacillus subtilis subspecies subtilis (Ehrenberg) Nakamura et al. strain DSM 4751, 1E34, BGSC 1E34, pAM77, Bacillus subtilis subspecies subtilis (Ehrenberg) Nakamura et al. strain DSM 4871, BD426, BGSC 1E21, pBD8, Bacillus subtilis subspecies subtilis (Ehrenberg) Nakamura et al. strain DSM 4872, BD466, BGSC 1E24, pBD10, Bacillus subtilis subspecies subtilis (Ehrenberg) Nakamura et al. strain DSM 4874, BGSC 1E38, pMK3, YB886, Bacillus subtilis subspecies subtilis (Ehrenberg) Nakamura et al. strain DSM 5213, BGSC 1A40, BR 151, Bacillus subtilis subspecies subtilis (Ehrenberg) Nakamura et al. strain DSM 5214, BD 393, BGSC 1A511, Bacillus subtilis subspecies subtilis (Ehrenberg) Nakamura et al. strain DSM 5545, BGSC 1A459/SU+III, Bacillus subtilis subspecies subtilis (Ehrenberg) Nakamura et al. strain DSM 5547, Bacillus subtilis (Ehrenberg) Cohn strain DSM 5552, Bacillus subtilis (Ehrenberg) Cohn strain DSM 5611, NRRL B-360, Bacillus subtilis subspecies subtilis (Ehrenberg) Nakamura et al. strain DSM 5660, NRRL B-362, Bacillus subtilis subspecies spizizenii Nakamura et al. strain DSM 6395, BGSC 2A2, W23 2A2, WB 672, Bacillus subtilis (Ehrenberg) Cohn strain DSM 6397, BGSC 1A2, SB 491, Bacillus subtilis subspecies spizizenii Nakamura et al. strain DSM 6399, BGSC 2A1, SB 623 Bacillus subtilis subspecies spizizenii Nakamura et al. strain DSM 6405, BGSC 2A3, W23 SR, Bacillus subtilis subspecies subtilis (Ehrenberg) Nakamura et al. strain DSM 6887, BGSC 1A309, NP40, Bacillus subtilis subspecies subtilis (Ehrenberg) Nakamura et al. strain DSM 6889, 1A658, BGSC 1A658, DA 65 Bacillus subtilis subspecies spizizenii Nakamura et al. strain DSM 8439, CCM 2268, IAM 12021, Bacillus subtilis (Ehrenberg) Cohn strain DSM 13019, SSI MK1, Bacillus subtilis subspecies spizizenii Nakamura et al. strain DSM 15029, NRRL B-23049, Bacillus subtilis subspecies inaquosorum Rooney et al. strain DSM 21200, Bacillus subtilis (Ehrenberg) Cohn strain DSM 21393, Bacillus subtilis subspecies inaquosorum Rooney et al. strain DSM 22148, KCTC 13429, Bacillus subtilis (Ehrenberg) Cohn strain DSM 23521, Bacillus subtilis (Ehrenberg) Cohn strain DSM 23778, Bacillus subtilis (Ehrenberg) Cohn strain DSM 25152, Bacillus subtilis (Ehrenberg) Cohn strain DSM 28592, Bacillus subtilis (Ehrenberg) Cohn strain DSM 30512, Bacillus subtilis (Ehrenberg) Cohn strain DSM 30529, Bacillus subtilis (Ehrenberg) Cohn strain DSM 30533, Bacillus subtilis (Ehrenberg) Cohn strain DSM 30534, Bacillus subtilis (Ehrenberg) Cohn strain DSM 30540, Bacillus subtilis (Ehrenberg) Cohn strain DSM 30541, Bacillus subtilis (Ehrenberg) Cohn strain DSM 30551, Bacillus subtilis (Ehrenberg) Cohn strain DSM 30558, Bacillus subtilis (Ehrenberg) Cohn strain DSM 30562, Bacillus subtilis (Ehrenberg) Cohn strain DSM 30570, Bacillus subtilis (Ehrenberg) Cohn strain DSM 30581, Bacillus subtilis (Ehrenberg) Cohn strain DSM 30597, Bacillus subtilis (Ehrenberg) Cohn strain DSM 30642, Bacillus subtilis (Ehrenberg) Cohn strain DSM 30651, Bacillus subtilis (Ehrenberg) Cohn strain DSM 30652, Bacillus subtilis (Ehrenberg) Cohn strain DSM 30671, Bacillus subtilis (Ehrenberg) Cohn strain DSM 30676, Bacillus subtilis (Ehrenberg) Cohn strain DSM 30677, Bacillus subtilis (Ehrenberg) Cohn strain DSM 30682, Bacillus subtilis (Ehrenberg) Cohn strain DSM 30711, Bacillus subtilis (Ehrenberg) Cohn strain DSM 30723, Bacillus subtilis (Ehrenberg) Cohn strain DSM 30801, Bacillus subtilis (Ehrenberg) Cohn strain DSM 30924, Bacillus subtilis (Ehrenberg) Cohn strain DSM 30925, Bacillus subtilis (Ehrenberg) Cohn strain DSM 30927, Bacillus subtilis (Ehrenberg) Cohn strain DSM 30928, Bacillus subtilis (Ehrenberg) Cohn strain DSM 30929, Bacillus subtilis (Ehrenberg) Cohn strain DSM 30941, D1, Bacillus subtilis (Ehrenberg) Cohn strain DSM 30942, D-FC1, Bacillus subtilis (Ehrenberg) Cohn strain DSM 31008, Bacillus subtilis (Ehrenberg) Cohn strain DSM 31009, Bacillus subtilis (Ehrenberg) Cohn strain DSM 31010, Bacillus subtilis (Ehrenberg) Cohn strain DSM 31020, Bacillus subtilis (Ehrenberg) Cohn strain DSM 31021, Bacillus subtilis (Ehrenberg) Cohn strain DSM 31033, Bacillus subtilis (Ehrenberg) Cohn strain DSM 100605, Bacillus subtilis (Ehrenberg) Cohn strain DSM 100612, Bacillus subtilis (Ehrenberg) Cohn strain DSM 100613, Bacillus subtilis (Ehrenberg) Cohn strain DSM 100614, Bacillus subtilis (Ehrenberg) Cohn strain DSM 103044, Bacillus subtilis (Ehrenberg) Cohn strain DSM 103047, Bacillus subtilis (Ehrenberg) Cohn strain DSM 103051, Bacillus subtilis (Ehrenberg) Cohn strain DSM 103758, Bacillus subtilis AM0904 (NRRL Deposit Number B-50914), Bacillus subtilis AM0911 (NRRL Deposit Number B-50915), Bacillus subtilis NP122 (NRRL Deposit Number B-50910), Bacillus subtilis NP119B (NRRL Deposit Number B-50909), Bacillus subtilis BS18 (NRRL B-50633), Bacillus subtilis BS278 (NRRL 50634), Bacillus subtilis 4-7d (NRRL B-50505), Bacillus subtilis 3-5h (NRRL B-50507), Bacillus subtilis AGTP BS3BP5 (NRRL B-50510), Bacillus subtilis BS918 (NRRL B-50508), Bacillus subtilis AGTP BS1013 (NRRL-50509), Bacillus subtilis AGTP 944 (NRRL B-50548), Bacillus subtilis AGTP BS442 (NRRL B-50542), Bacillus subtilis AGTP BS1069 (NRRL B-50544), Bacillus subtilis AGTP BS521 (NRRL B-50545), Bacillus subtilis B27 (NRRL B-50105), Bacillus subtilis 3A-P4 (PTA-6506), Bacillus subtilis 22C-P1 (PTA-6508), Bacillus subtilis BL21 (NRRL B-50134), Bacillus subtilis strain GB03, Bacillus subtilis strain QST713, Bacillus subtilis DSM 5750 (BioPlus® 2B, Chr. Hansen Bio Systems), Bacillus subtilis strain OBT 1224 (Osprey Biotechnic).

### Bacillus amyloliquefaciens Strains

Bacillus amyloliquefaciens (Fukumoto) Priest et al. (ATCC® 23350™), Bacillus amyloliquefaciens (Fukumoto) Priest et al. (ATCC® 23842™), Bacillus amyloliquefaciens SB 3296 (PTA-7548), Bacillus amyloliquefaciens (Fukumoto) Priest et al. (ATCC® 23843™), Bacillus amyloliquefaciens SB3297 (PTA-7549), Bacillus amyloliquefaciens (Fukumoto) Priest et al. (ATCC® BAA-390™), Bacillus amyloliquefaciens (Fukumoto) Priest et al. (ATCC® 23845™), Bacillus amyloliquefaciens (Fukumoto) Priest et al. (ATCC® 23844™), Bacillus amyloliquefaciens (Fukumoto) Priest et al. (ATCC® 31592™), Bacillus amyloliquefaciens (Fukumoto) Priest et al. (ATCC® 53495™), Bacillus amyloliquefaciens (Fukumoto) Priest et al. (ATCC® 49763™), Bacillus amyloliquefaciens: SB 3276 (PTA-7541), Bacillus amyloliquefaciens: PMBP-M7 (vial labeled BCRC PMBP-M7) (PTA-5819), Bacillus amyloliquefaciens SB 3284 (PTA-7545), Bacillus amyloliquefaciens SB 3288 (PTA-7546), Bacillus amyloliquefaciens MF215 (SB3446) (PTA-7790), Bacillus amyloliquefaciens SB 3283 (PTA-7544), Bacillus amyloliquefaciens MF 225 (SB 3448) (PTA-7791), Bacillus sp. (ATCC® 70038™, Deposited As Bacillus amyloliquefaciens (Fukumoto) Priest et al.), Bacillus amyloliquefaciens TOA5001 (NITE Patent Microorganisms Depositary Accession Number BP-01844).

ii) Amounts of the Different Bacillus Strains in the Bacillus Combinations

The relative amounts of Bacillus coagulans, Bacillus subtilis, Bacillus licheniformis and/or Bacillus amyloliquefaciens present in the Bacillus combination are selected to obtain a desired result. For certain embodiments, the Bacillus combination comprises from about 10⁵ to about 10¹² CFU/gram, and more typically from about 10⁵ to 10¹⁰ or from 10⁸ to 10¹⁰ CFU/gram of each of the Bacillus species in the Bacillus combination.

In some embodiments, the disclosed combination and/or composition is added to feed in an amount sufficient to provide from 1×10⁵ to 1×10⁷ CFU of the bacillus combination per gram of feed, such as from 2.5×10⁵ to 5×10⁶ CFU, from 5×10⁵ to 1.2×10⁶ CFU, or from 5×10⁵ to 8×10⁵ CFU of the Bacillus combination per gram of the feed.

In one embodiment, the disclosed combination and/or composition is added to feed in an amount sufficient to provide from 500,000 CFU to 1M CFU of the bacillus combination per gram of feed.

In one embodiment, the disclosed combination and/or composition is added to feed in an amount sufficient to provide 1M (1×10⁶) CFU of the bacillus combination per gram of feed.

In one embodiment, the disclosed combination and/or composition is added to feed in an amount sufficient to provide from 500,000 to 600,000 (5×10⁵ to 6×10⁵) CFU of the bacillus combination per gram of feed.

In other embodiments, the disclosed combination and/or composition is added to feed in an amount sufficient to provide from 45 g/ton (2000 pounds) of feed to 500 g/ton of feed, such as from 50 g/ton of feed to 450 g/ton of feed, from 100 g/ton of feed to 350 g/ton of feed, from 100 g/ton of feed to 300 g/ton of feed, or from 100 g/ton of feed to 250 g/ton of feed.

And in some embodiments, the disclosed combination and/or composition is administered in an amount sufficient to provide from 0.1 pounds to 1 pound of the Bacillus combination per ton of feed, such as from 0.1 pounds to 0.5 pounds of the Bacillus combination, from 0.2 pounds to 0.3 pounds of the Bacillus combination, or 0.25 pounds of the Bacillus combination per ton of feed. In other embodiments, the disclosed combination and/or composition is administered in an amount sufficient to provide from 5×10⁵ to 2.5×10⁶ CFU/gram of feed.

In some embodiments, the Bacillus combination may be administered to provide different CFU ratios of the Bacillus species included therein. In some embodiments, the ratio of Bacillus subtilis:Bacillus licheniformis in the Bacillus combination may be from 2:1 to 1:2, and typically is about 1:1, relative to each other. And with respect to other Bacillus species in the Bacillus combination, the total amount of Bacillus subtilis and Bacillus licheniformis (BSBL) relative to the other Bacillus species may be from greater than zero to 99%, such as from 10% to 90%, from 15% to 85%, from 20% to 80%, from 25% to 75%, from 35% to 65%, from 45% to 55%, or substantially 50%, based on CFU.

In some embodiments, the ASL combination may comprise, consist essentially of, or consist of, in amounts relative to each other, from 25% or less to 75% or more Bacillus amyloliquefaciens (BA) and from 75% or more to 25% or less BSBL. In certain embodiments, the ratio of BA to BSBL in the ASL combination is from 25%:75% BA:BSBL to 75%:25% BA:BSBL, and may be about 50%:50% BA:BSBL.

In some embodiments, the ASLC combination may comprise, consist essentially of, or consist of, in amounts relative to each other, from 25% or less to 75% or more in total of Bacillus amyloliquefaciens (BA) and Bacillus coagulans (BC), and from 75% or more to 25% or less BSBL. In certain embodiments, the ratio of BA+BC to BSBL in the ASL combination is from 25%:75% BA+BC:BSBL to 75%:25% BA+BC:BSBL, and may be about 50%:50% BA+BC:BSBL. The amounts of BA and BC, relative to each other may be from greater than zero to 99% BA relative to BC, such as from 10% to 90%, from 15% to 85%, from 20% to 80%, from 25% to 75%, from 35% to 65%, from 45% to 55%, or substantially 50% BA relative to BC, based on CFU.

For example, the CSL combination may comprise from 3.5×10⁹ to 10×10⁹ CFU Bacillus coagulans per gram of the CSL combination, such as from 4.1×10⁹ to 7.5×10⁹, from 5×10⁹ to 6.4×10⁹ or from 5×10⁹ to 6×10⁹ CFU Bacillus coagulans/gram. The CSL combination may comprise from 5×10⁸ to 10×10⁸ CFU Bacillus subtilis per gram of the CSL combination, such as from 6×10⁸ to 8.7×10⁸, from 6.9×10⁸ to 9×10⁸, or 7.2×10⁸ to 8×10⁸ CFU Bacillus subtilis/per gram. And the CSL combination may comprise from 5×10⁸ to 10×10⁸ CFU Bacillus licheniformis per gram of the CSL combination, such as from 6×10⁸ to 8.7×10⁸, from 6.9×10⁸ to 9×10⁸, or 7.2×10⁸ to 8×10⁸ CFU Bacillus licheniformis per gram.

In certain embodiments, the CSL combination may be administered to provide different CFU ratios of the three Bacillus species. For example, in one embodiment, the CSL combination ratio provides from about 6 parts to about 10 parts Bacillus coagulans to 1 part to 2 parts Bacillus subtilis, and from about 1 part to about 2 parts Bacillus licheniformis. The ratio of Bacillus subtilis:Bacillus licheniformis in the CSL combination may be from 2:1 to 1:2, and typically is about 1:1. In certain embodiments, the CSL combination comprises about 5×10⁹ Bacillus coagulans, about 8×10⁸ Bacillus subtilis, and 8×10⁸ Bacillus licheniformis per gram of the CSL combination.

In other embodiments, the Bacillus combination comprises, consists essentially of, or consists of, Bacillus subtilis, Bacillus licheniformis, Bacillus amyloliquefaciens and Bacillus coagulans. The relative amounts of the Bacillus species in such embodiments are from 25% to 50% Bacillus subtilis, from 30% to 65% Bacillus licheniformis, from 5% to 30% Bacillus amyloliquefaciens and from greater than zero to 15% Bacillus coagulans, in amounts relative to each other, such as from 30% to 45% Bacillus subtilis, from 40% to 60% Bacillus licheniformis, from 10% to 25% Bacillus amyloliquefaciens and from 1% to 12% Bacillus coagulans. In certain embodiments, the Bacillus combination comprises, consists essentially of, or consists of, from 30% to 40% Bacillus subtilis, from 40% to 50% Bacillus licheniformis, from 10% to 20% Bacillus amyloliquefaciens and from 2% to 10% Bacillus coagulans in amounts relative to each other. In some embodiments, the amount of Bacillus licheniformis is greater than the amount of Bacillus subtilis in the Bacillus combination. In certain embodiments, such a composition can be a commercially available product, such as the composition sold as Provia Prime™ by Phibro Animal Health Corporation.

B. Essential Oil Composition

The essential oil composition may comprise, consist essentially of, or consist of, one or more essential oils, such as 1, 2, 3 or 4 essential oils, such as 1, 2, or 3 essential oils, or 1 or 2, essential oils, and in some embodiments, the essential oil composition comprises, consists essentially of, or consists of, one essential oil. In some embodiments, the essential oil composition comprises oregano essential oil. And in certain embodiments, the essential oil composition is oregano essential oil.

With respect to the essential oil composition, the term “consisting essentially of” means that the essential oil composition does not include any additional essential oils, but may include additional components suitable for preparing an essential oil composition, such as a solvent, vehicle, filler, preservative, stabilizer, or any combination thereof.

A person of ordinary skill in the art will appreciate that, as used herein, a plant name may refer to the plant as a whole, or to any part of the plant, such as the roots, stem or trunk, bark, leaves, flower, flower stems, seeds, or a combination thereof. These plant parts may be used fresh, or dried, and may be whole, pulverized, or comminuted. The plant name may also refer to extracts from any part or parts of the plant, such as chemical extracts, or extracts obtained by pressing, or any other methods of concentrating or extracting oils or other extracts known to those in the art or that are hereafter discovered. Plant extracts may include compounds that are saponins, triterpenoids, polyphenols, antioxidants or resveratrol, or combinations thereof.

In certain embodiments, the essential oil is oregano essential oil.

In some embodiments, the oregano essential oil comprises carvacrol and thymol, such as from 75 wt % to 95 wt %, or from 80 wt % to 90 wt % carvacrol, and from 0.7 wt % to 4 wt %, or from 1.5 wt % to 3 wt % thymol, as determined by gas chromatography-mass spectrometry (GC-MS) analysis. In certain embodiments, the oregano essential oil comprises carvacrol, thymol, γ-terpinene, p-cymene, β-caryophyllene, and optionally minor amounts of other terpenes, phenols, and/or residual solvent(s). In certain embodiments, the oregano essential oil comprises:
Carvacrol from 75 wt % to 95 wt %, such as from 80 wt % to 90 wt %;
Thymol from 0.7 wt % to 4 wt %, such as from 1.5 wt % to 3 wt %;
γ-Terpinene from 1 wt % to 5 wt %, such as from 1 wt % to 3 wt %;
p-Cymene from 2 wt % to 6 wt %, such as from 2 wt % to 4 wt %;
β-Caryophyllene from 0.2 wt % to 1.8 wt %, such as from 0.2 wt % to 1 wt %;

Other terpenes and/or phenols from zero to 18 wt %, such as from greater than zero to 18 wt %, from 0.1 wt % to 18 wt % or from 1 wt % to 5 wt %; and

Residual solvent(s) from greater than zero to 0.001 wt %, or from greater than zero to less than 0.001 wt %. In some embodiments, less than 0.001 wt % is below a detection limit.

A person of ordinary skill in the art understands that the amounts disclosed for each oregano essential oil component are relative to the amounts of the other components of the essential oil such that the total amount equals 100%.

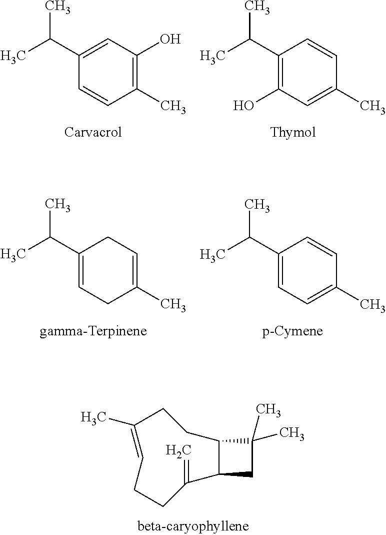

The disclosed combination and/or composition may be administered in an amount sufficient to provide the essential oil composition in an amount sufficient to provide from greater than zero to 500 ppm or more of essential oil per ton (2,000 pounds) of feed, such as from 25 ppm to 500 ppm, or from 50 ppm to 250 ppm of essential oil per ton of feed. In some embodiments, the disclosed combination and/or composition is administered in an amount sufficient to provide 50 ppm, 100 ppm, 150 ppm, 200 ppm or 250 ppm of essential oil per ton of feed. In one embodiment, 50 ppm oregano essential oil is used. In one embodiment, 250 ppm oregano essential oil is used.

In another embodiment, the disclosed combination and/or composition may be administered in an amount sufficient to provide the essential oil composition in an amount sufficient to provide from greater than zero to 500 grams or more of essential oil, such as oregano essential oil, per ton (2,000 pounds) of feed, such as from 25 grams to 500 grams, from 50 grams to 350 grams, from 100 grams to 300 grams, or from 100 grams to 250 grams of essential oil per ton of feed. In some embodiments, the disclosed combination and/or composition is administered in an amount sufficient to provide 50 grams, 100 grams, 150 grams, 200 grams or 250 grams of essential oil, such as oregano essential oil, per ton of feed. In one embodiment, 150 grams oregano essential oil is used. In one embodiment, 250 grams oregano essential oil is used.

### III. Yucca and/or Quillaja, or Extracts Thereof

Additionally, the disclosed Bacillus/essential oil combination and/or composition can be administered in combination with yucca and/or quillaja plant material, or extracts thereof. Examples of yucca include, but are not limited to, Yucca aloifolia, Yucca angustissima, Yucca arkansana, Yucca baccata, Yucca baileyi, Yucca brevifolia, Yucca campestris, Yucca capensis, Yucca carnerosana, Yucca cernua, Yucca coahuilensis, Yucca constricta, Yucca decipiens, Yucca declinata, Yucca de-smetiana, Yucca elata, Yucca endlichiana, Yucca faxoniana, Yucca filamentosa, Yucca filifera, Yucca flaccida, Yucca gigantean, Yucca glauca, Yucca gloriosa, Yucca grandiflora, Yucca harrimaniae, Yucca intermedia, Yucca jaliscensis, Yucca lacandonica, Yucca linearifolia, Yucca luminosa, Yucca madrensis, Yucca mixtecana, Yucca necopina, Yucca neomexicana, Yucca pallida, Yucca periculosa, Yucca potosina, Yucca queretaroensis, Yucca reverchonii, Yucca rostrata, Yucca rupicola, Yucca schidigera, Yucca schottii, Yucca sterilis, Yucca tenuistyla, Yucca thompsoniana, Yucca treculeana, Yucca utahensis, Yucca valida or combinations thereof. In certain embodiments, the Yucca is or comprises Yucca schidigera.

Examples of quillaja include, but are not limited to, Quillaja brasiliensis, Quillaja lanceolata, Quillaja lancifolia, Quillaja molinae, Quillaja petiolaris, Quillaja poeppigii, Quillaja saponaria, Quillaja sellowiana, Quillaja smegmadermos or combinations thereof. In certain embodiments, the quillaja is or comprises Quillaja saponaria.

A person of ordinary skill in the art will appreciate that, as used herein, a plant name may refer to the plant as a whole, or to any part of the plant, such as the roots, stem or trunk, bark, leaves, flower, flower stems, seeds, or a combination thereof. These plant parts may be used fresh, or dried, and may be whole, pulverized, or comminuted. The plant name may also refer to extracts from any part or parts of the plant, such as chemical extracts, or extracts obtained by pressing, or any other methods of concentrating or extracting oils or other extracts known to those in the art or that are hereafter discovered. Plant extracts may include compounds that are saponins, triterpenoids, polyphenols, antioxidants or resveratrol, or combinations thereof.

The combination may comprise a composition comprising yucca and/or quillaja that may also include carriers and binding agents suitable to formulate the yucca and/or quillaja for administration to an animal. In certain embodiments, such a composition can be a commercially available product, such as a composition comprising Yucca schidigera and Quillaja saponaria, sold under the trademark NUTRAFITO PLUS by Desert King International and/or MAGNI-PHI® by Phibro Animal Health Corporation. Such compositions may comprise from 99% or more Quillaja saponaria and 1% or less Yucca schidigera to 75% Quillaja saponaria and 25% Yucca schidigera, such as from 95% Quillaja saponaria and 5% Yucca schidigera to 80% Quillaja saponaria and 20% Yucca schidigera, and in certain embodiments, 85% Quillaja saponaria and 15% Yucca schidigera, or from 90% to 95% Quillaja saponaria and from 5% to 10% Yucca schidigera, such as from 92% to 93% Quillaja saponaria and from 7% to 8% Yucca schidigera, or about 92.5% Quillaja saponaria and about 7.5% Yucca schidigera.

In some embodiments, a combination and/or composition comprises, consists essentially of, or consists of, yucca, quillaja, Bacillus coagulans, Bacillus subtilis, Bacillus licheniformis, Bacillus amyloliquefaciens, and an essential oil, such as oregano essential oil. And in certain embodiments, the combination and/or composition comprises, consists essentially of, or consists of, Yucca schidigera, Quillaja saponaria, Bacillus coagulans, Bacillus subtilis, Bacillus licheniformis, Bacillus amyloliquefaciens and oregano essential oil.

The disclosed combination and/or composition may be administered in an amount sufficient to provide from 100 to 500 ppm or more of the yucca and quillaja combination per ton of feed, such as from 200 to 500 ppm yucca and quillaja, or from 200 to 400 ppm yucca and quillaja per ton of feed. In one embodiment, the disclosed combination and/or composition is administered in an amount sufficient to provide 200 ppm, 250 ppm, 300 ppm, 350 ppm, or 400 ppm yucca and quillaja per ton of feed.

In other embodiments, the disclosed combination and/or composition is administered in an amount sufficient to provide from 0.1 to 1 pound or more of the yucca and quillaja combination per ton of feed, such as from 0.1 pounds to 0.5 pounds yucca and quillaja per ton of feed, or from 0.2 pounds to 0.4 pounds yucca and quillaja per ton of feed.

In other embodiments, the disclosed combination and/or composition is administered in an amount sufficient to provide from 50 grams yucca and quillaja per ton of feed to 500 grams or more yucca and quillaja per ton of feed, such as from 75 grams yucca and quillaja per ton to 300 grams yucca and quillaja per ton of feed, from 100 grams yucca and quillaja per ton to 250 grams yucca and quillaja per ton of feed. In some embodiments, the disclosed combination and/or composition is administered in an amount sufficient to provide 110 grams to 115 grams yucca and quillaja per ton of feed. And in other embodiments, the disclosed combination and/or composition is administered in an amount sufficient to provide 240 grams to 260 grams yucca and quillaja per ton of feed.

In any embodiments, the yucca may be Yucca schidigera or an extract thereof, and the quillaja may be Quillaja Saponaria or an extract thereof.

The disclosed combination and/or composition may comprise, consist essentially of, or consist of, from 100 to 500 ppm or more yucca and quillaja, from 0.1 to 1 pounds of Bacillus coagulans, Bacillus subtilis, Bacillus licheniformis and Bacillus amyloliquefaciens, and from 50 ppm to 250 ppm essential oil per ton of a feed, such as from 200 to 500 ppm yucca and quillaja, from 0.1 to 0.5 pounds of Bacillus coagulans, Bacillus subtilis, Bacillus licheniformis and Bacillus amyloliquefaciens, and from 50 ppm to 250 ppm essential oil, and in certain embodiments, from 200 to 400 ppm Yucca schidigera and Quillaja saponaria, and from 0.2 to 0.3 pounds of Bacillus coagulans, Bacillus subtilis, Bacillus licheniformis and Bacillus amyloliquefaciens and from 50 ppm to 250 ppm oregano essential oil.

### IV. Additional Component(s)

The disclosed combination and/or composition also can be administered in combination with one or more additional components or compositions. An additional component or composition may be any component or composition that can be administered to a subject, particularly a non-ruminant animal, such as an avian, including poultry, in combination with the disclosed combination and/or composition. Certain disclosed embodiments of the combination and/or composition are particularly formulated for administration to poultry, and therefore can comprise the Bacillus combination, essential oil composition, and optionally yucca and/or quillaja composition, in combination with any other component or composition now known or hereafter developed for administration to poultry.

Exemplary additional components suitable for use with the disclosed combination and/or composition include a carrier, a vitamin, a copper salt, a feed supplement, an additional DFM, a feed, such as a poultry feed, or a combination thereof. The additional component(s) may comprise from 1 wt % to 99 wt % and the disclosed combination and/or composition comprising the Bacillus combination, essential oil composition, and optionally yucca and/or quillaja composition, may comprise from 99 wt % to 1 wt % of the total weight of the combination, such that the combined weight of the additional component(s) and the disclosed combination and/or composition is 100%. Preferably the additional component(s) will comprise from 10 wt % to 90 wt % and the disclosed combination and/or composition will comprise from 90 wt % to 10 wt % of the total weight of the combination. Yet even more preferably, the additional component(s) will comprise 20 wt % to 80 wt % and the disclosed combination and/or composition will comprise from 80 wt % to 20 wt % of the total weight of the combination. The disclosed combination and/or composition may be administered with the other component(s), optionally in a mixture with the other component(s), such as poultry feed and/or a feed supplement, in an amount sufficient to provide the desired amounts of the respective Bacillus species, essential oil composition, and optionally yucca and/or quillaja, in the particular combination. Exemplary additional components and/or compositions of a combination comprising the disclosed combination and/or composition are discussed in more detail below.

A. Carrier

In some embodiments, the disclosed combination and/or composition may be mixed with and/or dispersed in a carrier to form a dispersed composition. The carrier(s) may be selected to provide a non-biological benefit to the composition, compared to a disclosed combination and/or composition without a carrier, such as, but not limited to, achieving or improving a readily flowable state, and/or improving stability during storage and/or transport. Suitable carriers that may be used in combination with the disclosed combination and/or composition include, but are not limited to, plant material, such as beet pulp, ground corn, corn syrup solids, plant fiber, rice hulls, soluble plant fiber, wheat middlings, microcrystalline cellulose; carbonates, such as metal carbonates, such as calcium carbonate, potassium carbonate; sulfates, such as metal sulfates, such as potassium sulfate, sodium sulfate; lactates, including metal lactates, such as calcium lactate; oxides, including metal oxides, such as calcium oxide; propionates, including metal propionates, such as calcium propionate; stearates, including metal stearates, such as calcium stearate; phosphates, such as dicalcium phosphate dehydrate, monocalcium phosphate, sodium tripolyphosphate, or tetra sodium pyrophosphate; minerals, such as dolomite, silicon dioxide, silica, limestone, or vermiculite; clays, such as bentonite, montmorillonite, kaolin; sugars, such as glucose, sucrose, dextrose, fructose, or a combination thereof; maltodextrin; salt, such as sodium chloride; carrageenan; cellulose; guar gum; polyols; sodium alumino silicate; urea; animal protein products; forage products; grain products; plant protein products; processed grain products; roughage products; molasses products; or combinations thereof. In certain embodiments, the carrier is calcium carbonate.

Animal protein products may include, but are not limited to, blood meal; animal by-product meal; buttermilk, including condensed buttermilk and dried buttermilk; casein; dried hydrolyzed casein; cheese rind; crab meal; fish products, including fish by-products, fish liver and glandular meal, fish meal, fish protein concentrates, fish residue meal, and dried and/or condensed fish solubles; fleshings hydrolysate; hydrolyzed hair; hydrolyzed leather meal; hydrolyzed poultry by-product aggregate; hydrolyzed poultry feathers; leather hydrolysate; meat and bone meal; meat and bone meal tankage; meat meal; meat meal tankage; dried meat solubles; dried lactalbumin; dried feed grade milk; dried milk protein; poultry by-products and/or by-products meal; poultry hatchery by-product; shrimp meal; skimmed milk, including condensed, condensed cultured, dried, or dried cultured skimmed milk; whey, including condensed, condensed cultured, condensed hydrolyzed, dried, or dried hydrolyzed whey; condensed and/or dried whey product; condensed and/or dried whey solubles; or a combination thereof.

Forage products may include, but are not limited to, alfalfa products, such as dehydrated meal, optionally in pellet form, ground hay, or suncured meal, optionally in pellet form; coastal bermudagrass hay; dehydrated corn plant; dehydrated silage; flax plant product; ground grass; lespedeza meal and/or stem meal; ground soybean hay; or combinations thereof.

Grain products may include, but are not limited to, barley, corn, grain sorghum, mixed feed oats, oats, triticale, wheat, ground brown rice, ground or ground paddy rough rice, broken or chipped rice, brewers rice, rye, or a combination thereof. The grain products may be in any suitable form, such as whole, ground, cracked, hulls, bran, screen cracked, flaked, kibbled, toasted, and/or heat processed.

Plant protein products may include, but are not limited to, dried beans; canola meal; coconut meal; cottonseed, such as flakes, cake, meal, low gossypol meal, and/or whole pressed cottonseed; guar meal; dried kelp; linseed meal; peanut meal; peas; potato protein; dried seaweed meal; safflower meal; soy protein concentrate; soybean feed; ground soybeans; soybean meal, optionally kibbled; heat processed soybeans; ground, extruded whole soybeans; soy flour; soy grits; sunflower meal, optionally dehulled; or a combination thereof.

The processed grain by-products may be aspirated grain fractions; brewers dried grains; buckwheat middlings; condensed distillers solubles; condensed fermented corn extracts; corn bran; corn flour; corn germ meal; corn gluten feed and/or meal; corn grits; distillers dried grains, optionally with solubles; distillers dried solubles, flour, grain sorghum germ cake, meal, grits, and/or mill feed; meal hominy feed; malt sprouts; oat groats; feeding oat meal; pearl barley by-product; peanut skins; rice bran; rice polishings; rye middlings; gelatinized or partially aspirated sorghum grain flour; wheat bran, flour, shorts, germ meal, defatted germ meal, middlings, mill run and/or red dog; or a combination thereof.

Roughage products may include, but are not limited to, almond hulls; dried apple pectin pulp; dried apple pomace; bagasse; barley hulls; barley mill by-product; dried, plain beet pulp; buckwheat hulls; dried citrus meal; dried citrus pulp; citrus seed meal; corn cob fractions; cottonseed hulls; flax straw by-product; ground corn cob; psyllium seed husk; malt hulls; clipped oat by-product; oat hulls; oat mill by-product; peanut hulls; rice hulls; rice mill by-product; rye mill run; soybean hulls, mill feed, and/or mill run; sunflower hulls; ground straw; dried tomato pomace; or a combination thereof.

Molasses products may be beet molasses; dried beet molasses product; dried beet pulp molasses; cane molasses; citrus molasses; molasses yeast condensed solubles; concentrated separator by-product; condensed molasses fermentation solubles; starch molasses; molasses distillers condensed solubles; molasses distillers dried solubles; or a combination thereof.

B. Copper Species

The disclosed combination and/or composition may be mixed with a copper species such as a copper species that provides a copper ion. The copper species may be a copper salt. Exemplary copper species that may be combined with the disclosed combination and/or composition include, but are not limited to, copper chloride, copper bromide, copper iodide, copper sulfate, copper sulfite, copper bisulfite, copper thiosulfate, copper phosphate, monobasic copper phosphate, dibasic copper phosphate, copper hypophosphite, copper dihydrogen pyrophosphate, copper tetraborate, copper borate, copper carbonate, copper bicarbonate, copper metasilicate, copper citrate, copper malate, copper methionate, copper succinate, copper lactate, copper formate, copper acetate, copper butyrate, copper propionate, copper benzoate, copper tartrate, copper ascorbate, copper gluconate, or a combination thereof, preferably copper sulfate, copper acetate, copper citrate, copper methionate, or a combination thereof. A copper species, such as a copper salt, may be provided separately, or individually, or it may be provided as part of a composition, such as a feed or a feed supplement. Certain disclosed embodiments comprise, consist essentially of, or consist of the disclosed combination and/or composition and a copper species. In any embodiments, the copper species may be a copper salt, such as a salt that can provide a copper ion, for example, copper sulfate.

C. Vitamin(s)

Exemplary vitamins include, but are not limited to, one or more of Vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (including folic acid), Vitamin B12 (various cobalamins; commonly cyanocobalamin in vitamin supplements), Vitamin C (ascorbic acid or a salt thereof, such as sodium ascorbate or calcium sorbate), Vitamin D (vitamin D₁, vitamin D₂, vitamin D₃, vitamin D₄, vitamin D₅, 25-hydroxy vitamin D₃, 25-dihydroxy vitamin D₃, or combinations thereof), Vitamin E, Vitamin K (K1 and K2 (i.e. MK-4, MK-7)), and biotin, and derivatives, salts and/or analogs thereof.

D. Feed

The feed may be any feed suitable for administration to a non-ruminant animal. The disclosed combination and/or composition may be administered in combination with the feed, such as by forming a mixture of the disclosed combination and/or composition and the feed, or by administering the disclosed combination and/or composition and the feed sequentially, in any order. In certain disclosed embodiments the animal is a poultry, and the disclosed combination and/or composition is used in combination with, and may be admixed with, a poultry feed, such as a poultry basal diet. The feed may comprise corn, alfalfa, peas, soybean meal, soybean oil, wheat, oats, sorghum, barley, rye, rice hulls, canola, corn oil, limestone, salt (for example, sodium chloride), distillers dried grains with solubles (DDGS), dicalcium phosphate, sodium sesquicarbonate, methionine source, lysine source, L-threonine, mineral oil, biotin, folic acid, kelp, menadione dimethylpyrimidinol bisulfite, calcium aluminosilicate, or any combination thereof. The feed may also comprise one or more additional components. Additional components may be used for any desired purpose, such as a substantially biologically inert material added, for example, as a filler, or to provide a desired beneficial effect. For example, the feed may include a carbonate (including a metal carbonate such as calcium carbonate); a trace mineral, such as, but not limited to, chloride, fluoride, iodide, chromium, copper, zinc, iron, magnesium, manganese, molybdenum, phosphorus, potassium, sodium, sulfur, selenium, or a combination thereof; a bulking agent; a carrier; a colorant; a taste enhancer; a preservative; one or more vitamins; or a combination thereof. The preservative may be benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof. The one or more vitamins may include vitamin A; vitamin B1, such as thiamine mononitrate; vitamin B₂, such as riboflavin-5-phosphate; vitamin B₃, such as niacin or niacinamide; vitamin B₅, such as pantothenic acid or d-calcium pantothenate; vitamin B₆, such as pyridoxine or pyridoxine hydrochloride; vitamin B₁₂; vitamin C, such as ascorbic acid, sodium ascorbate, or calcium sorbate; vitamin D; vitamin E; vitamin K, or a combination thereof. Vitamin D may comprise vitamin D₁, vitamin D₂, vitamin D₃, vitamin D₄, vitamin D₅, 25-hydroxy vitamin D₃, 25-dihydroxy vitamin D₃, or combinations thereof.

The feed, such as a poultry feed, may also include fats and/or oils, such as tallow, optionally derived from the rendering of beef offal; lard, optionally derived from the rendering of pork offal; poultry fat, optionally derived from poultry offal; feed grade animal fat, optionally derived from a mixture of rendered beef, pork, and/or poultry raw material; yellow grease, optionally derived from reprocessed restaurant grease and/or cooking oil; and/or blended animal-vegetable fat, which may include blends of different types and/or amounts of animal fats and vegetable oils from restaurant grease. Additionally, or alternatively, the feed may include protein sources, such as canola, fish meal, field peas, meat and bone meal, soybeans, and/or cereal by-products.

E. Silica, Mineral Clay, Glucan and Mannans

Additionally, or alternatively, the disclosed combination and/or composition can be administered in combination with a feed supplement comprising silica, mineral clay, glucan and mannans. The feed supplement may further comprise an endoglucanohydrolase, either endogenously or as an affirmatively added ingredient. As used herein, weight % for endoglucanohydrolase is based on a 70,000 unit/gram endoglucanohydrolase product. The endoglucanohydrolase may be β-1,3 (4)-endoglucanohydrolase.

In any embodiments disclosed herein, the feed supplement may comprise, consist essentially of, or consist of, glucan (e.g., β-1,3 (4)glucan), silica, mineral clay and mannans. In some embodiments, the feed supplement comprises, consists essentially of, or consists of, glucan (e.g., β-1,3 (4)glucan), silica, mineral clay, mannans and endoglucanohydrolase. In any embodiments disclosed herein, the glucan and mannans may be provided, at least in part, by yeast cell wall or an extract thereof. Thus, in some embodiments, the feed supplement may comprise, consist essentially of, or consist of, silica, mineral clay and yeast cell wall or an extract thereof, or the feed supplement may comprise, consist essentially of, or consist of, silica, mineral clay, yeast cell wall or an extract thereof, and endoglucanohydrolase. Similarly, endoglucanohydrolase may, in certain disclosed embodiments, be provided by yeast cell wall or a yeast cell wall extract.

Suitable sources of silica include, but are not limited to, sand, diatomaceous earth, and synthetic silica. In one embodiment, quartz may be used. In certain embodiments, the mannans comprise glucomannan.

The components of the feed supplement are prepared by methods commonly known in the art and can be obtained from commercial sources. β-1,3 (4)-endoglucanohydrolase may be produced from submerged fermentation of a strain of Trichoderma longibrachiatum. Diatomaceous earth is available as a commercially-available product with from 70% to 95% silica (SiO₂) and with its remaining components not assayed but primarily ash (minerals) as defined by the Association of Analytical Chemists (AOAC, 2002). The mineral clays (e.g., aluminosilicates) used in this feed supplement may be any of a variety of commercially-available clays including, but not limited to, montmorillonite clay, bentonite and zeolite. Glucan, mannans, and/or endoglucanohydrolase can be obtained from plant cell walls, yeast or yeast cell wall or an extract thereof (e.g., Saccharomyces cerevisiae, Candida utilis), certain fungi (e.g., mushrooms), algae, and bacteria. In certain embodiments, yeast can be administered affirmatively to provide glucan, mannans and endoglucanohydrolase endogenously.

In one embodiment, the feed supplement comprises, consists essentially of, or consists of, 1-40 wt % silica, 0.5-25 wt % glucan and mannans, and 40-92 wt % mineral clay, in amounts relative to each other. In another embodiment, the feed supplement comprises, consists essentially of, or consists of, 5-40 wt % silica, 0.5-15 wt % glucan and mannans, and 40-80 wt % mineral clay, in amounts relative to each other. In another embodiment, the feed supplement comprises, consists essentially of, or consists of, 20-40 wt % silica, 0.5-10 wt % glucan and mannans, and 50-70 wt % mineral clay, in amounts relative to each other. In another embodiment, the feed supplement comprises, consists essentially of, or consists of, 15-40 wt % silica, greater than zero to 15 wt % glucans, greater than zero to 10 wt % mannans, and 50-81 wt % mineral clay, in amounts relative to each other. In another embodiment, the feed supplement comprises, consists essentially of, or consists of, 15-40 wt % silica, 0.5-5.0 wt % glucans, 0.5-8.0 wt % mannans, and 50-81 wt % mineral clay, in amounts relative to each other. In another embodiment, the feed supplement comprises, consists essentially of, or consists of, 20-30 wt % silica, 0.5-3.5 wt % glucans, 0.5-6.0 wt % mannans, and 60-70 wt % mineral clay, in amounts relative to each other.

In some embodiments, β-glucans and mannans are obtained from yeast or yeast cell wall or an extract thereof. The feed supplement may comprise, consist essentially of, or consist of, 1-40 wt % silica, 1-30 wt % yeast cell wall or an extract thereof, and 40-92 wt % mineral clay, in amounts relative to each other. In one embodiment, the feed supplement comprises, consists essentially of, or consists of, 10-40 wt % silica, 5-20 wt % yeast cell wall or an extract thereof, and 40-80 wt % mineral clay, in amounts relative to each other. In another embodiment, the feed supplement comprises, consists essentially of, or consists of, 15-30 wt % silica, 5-15 wt % yeast cell wall or an extract thereof, and 50-70 wt % mineral clay, in amounts relative to each other.

In any of the above embodiments, the feed supplement may further comprise an endoglucanohydrolase, such as β-1,3 (4)-endoglucanohydrolase. The feed supplement may include from 0.025 wt % endoglucanohydrolase to 5 wt % endoglucanohydrolase or more, such as from 0.05 wt % to 3 wt % β-1,3 (4)-endoglucanohydrolase, relative to the amounts of silica, mineral clay, glucan, mannans, and/or yeast, yeast cell wall, or yeast cell wall extract present in the feed supplement. In one embodiment, the feed supplement comprises, consists essentially of, or consists of, 0.1-3 wt % β-1,3 (4)-endoglucanohydrolase, 20-40 wt % silica, 0.5-20 wt % glucan and mannans, and 50-70 wt % mineral clay, in amounts relative to each other. In another embodiment, the feed supplement comprises, consists essentially of, or consists of, 0.1-3 wt %, β-1,3 (4)-endoglucanohydrolase, 20-40 wt % silica, 0.5-10 wt % glucan and mannans, and 50-70 wt % mineral clay, in amounts relative to each other. Alternatively, the feed supplement may comprise, consist essentially of, or consist of, 0.1-3 wt % β-1,3 (4)-endoglucanohydrolase, 1-40 wt % silica, 5-30 wt % yeast cell wall or an extract thereof, and 40-92 wt % mineral clay, in amounts relative to each other. In one embodiment, the feed supplement comprises, consists essentially of, or consists of, 0.1-3 wt % β-1,3 (4)-endoglucanohydrolase, 10-40 wt % silica, 5-20 wt % yeast cell wall or an extract thereof, and 40-80 wt % mineral clay, in amounts relative to each other. In another embodiment, the feed supplement comprises, consists essentially of, or consists of, 0.1-3 wt % β-1,3 (4)-endoglucanohydrolase, 15-30 wt % silica, 5-15 wt % yeast cell wall or an extract thereof, and 50-70 wt % mineral clay, in amounts relative to each other.

In any of the above embodiments, the silica may be provided by diatomaceous earth. In any of the above embodiments, the glucans may be β-glucans. In some embodiments, the β-glucans can be obtained from yeast, or other materials, such as fungi, algae, bacteria, or the like. In any of the above embodiments, the mannans may comprise glucomannan.

The glucan and mannans (or yeast or yeast cell wall or an extract thereof) can be prepared by a method known to a person of ordinary skill in the art and as further disclosed by the patent documents incorporated herein by reference. Yeast cell wall or an extract thereof may have a feed supplement comprising 0-15% moisture and 85-100% dry matter. The dry matter may comprise 10-65% protein, 0-25% fats, 0-3% phosphorus, 5-30% β-glucan, 5-35% mannans, and 0-15% ash. In an independent embodiment, a commercial source of β-1,3 (4) glucan and glucomannan derived from primary inactivated yeast (Saccharomyces cerevisiae) with the following chemical feed supplement can be used: moisture 2-5%; proteins 40-50%; fats 3-8%; phosphorus 0-2%; mannans 10-16%; β-1,3-(4) glucan 10-20%; and ash 2-12%.

In another independent embodiment, the yeast cell wall or an extract thereof comprises moisture 1-7% and dry matter 93-99%, and the dry matter may comprise proteins 18-28%, fats 10-17%, phosphorus 0-2%, mannans 20-30%, β-1,3-(4) glucan 18-28%, and ash 2-5%.

In an independent embodiment of the feed supplement, silica, glucan and mannans, and mineral clay are combined at 1-40%, 0.5-25% and 40-92% by weight, respectively. In an independent embodiment of the feed supplement and/or combination, β-1,3 (4)-endoglucanohydrolase, diatomaceous earth, yeast cell wall or an extract thereof, and mineral clay are combined at 0.05-3%, 1-40%, 1-20% and 40-92% by weight, respectively. In an independent feed supplement and/or combination, β-1,3 (4)-endoglucanohydrolase, diatomaceous earth, yeast cell wall or an extract thereof, and mineral clay are combined at 0.1-3%, 5-40%, 2-15% and 40-80% by weight, respectively. In another independent embodiment of the feed supplement and/or combination, β-1,3 (4)-endoglucanohydrolase, diatomaceous earth, yeast cell wall or an extract thereof, and mineral clay are combined at 0.1-3%, 30-40%, 4-15% and 50-65% by weight, respectively.

The feed supplement may further comprise one or more additional components. Additional components may be used for any desired purpose, such as a substantially biologically inert material added, for example, as a filler, or to provide a desired beneficial effect. For example, the feed supplement may include a carbonate (including a metal carbonate such as calcium carbonate); a trace mineral, such as, but not limited to, chloride, fluoride, iodide, chromium, copper, zinc, iron, magnesium, manganese, molybdenum, phosphorus, potassium, sodium, sulfur, selenium, or a combination thereof; a bulking agent; a micro tracer, such as iron particles coated with a dye; yeast; allicin; alliin; allinase; algae; a polyphenol or plant material comprising polyphenol; a carrier; a colorant; a taste enhancer; a preservative; an oil; a vitamin; a sorbic acid or a salt thereof; or a combination thereof. The yeast may be yeast culture, active yeast, a live yeast, a dead yeast, yeast extract, active dried yeast, brewers dried yeast, culture yeast, dried yeast, primary dried yeast, torula dried yeast, candida dried yeast, or a combination thereof. The preservative may be benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof. The oil may be mineral oil, corn oil, soybean oil, or a combination thereof. The sorbic acid or salt thereof may be potassium sorbate, sodium sorbate, ammonium sorbate, or a combination thereof. The vitamin may be vitamin A, vitamin B1, vitamin B₂, vitamin B₃, vitamin B₅, vitamin B₆, vitamin B₁₂, vitamin C, vitamin D, vitamin E, vitamin K, or a combination thereof.

Allicin (diallyl thiosulfate; 2-Propene-1-sulfinothioic acid S-2-propenyl ester) is a compound found in garlic, such as raw garlic. Allicin is typically produced from alliin ((2R)-2-amino-3-[(S)-prop-2-enylsulfinyl]propanoic acid) in damaged garlic cells by the action of the enzyme alliinase. Allicin, alliin, and/or alliinase may be provided as whole garlic cloves or bulbs; crushed, mashed, or chopped garlic; a garlic extract; and/or as a synthesized or isolated compound.

The polyphenol may be provided by a plant extract from a polyphenol-containing plant material. The plant material also may include non-polyphenol compounds, including polyphenol degradation products, such as gallic acid and trans-caftaric acid. Degradation can occur, for example, through oxidative and/or biological processes. Both the polyphenols and the non-polyphenol compounds may have biological activity. The plant extract may be prepared from a single plant material or from a combination of plant materials. Suitable plant materials from which a plant extract can be obtained include, but are not limited to, apples, blackberries, black chokeberries, black currants, black elderberries, blueberries, cherries, cranberries, grapes, green tea, hops, onions, quillaja, plums, pomegranates, raspberries, strawberries, and yucca.

In some embodiments, the plant extract is prepared from a pressed plant material, such as grape pomace, a dried plant material, such as tea, or a combination thereof. Pomace may be obtained substantially immediately post-pressing or as an ensiled product, i.e., pomace collected and stored for up to several months post-pressing. Suitable plants have a plurality of polyphenols and/or other non-polyphenolic compounds including, but not limited to, non-polyphenolic organic acids (such as gallic acid and/or trans-caftaric acid), flavanols, gallate esters, flavanodiols, phloroglucinol, pyrogallol, and catechol. In some embodiments, the plant extract is prepared from Pinot noir pomace, Pinot gris pomace, or green tea.

In some embodiments, pressed or dried plant material is ground to a fine powder prior to, or during, extraction. Pressed plant materials may be frozen to facilitate grinding. Polyphenols and other non-polyphenolic compounds may be extracted for administration. For example, polyphenols and other non-polyphenolic compounds may be extracted from the powder using a solution comprising a polar solvent, such as water, an alcohol, an ester, or a combination thereof. In some embodiments, the solution comprises a water-miscible alcohol, ester, or combination thereof, such as a lower alkyl alcohol, lower alkyl ester, or a combination thereof. In some embodiments, the solution is water or an aqueous solution comprising 25-99% solvent, such as 25-95% solvent, 30-80% solvent, or 50-75% solvent, and water. In certain embodiments, the solution is an aqueous solution comprising methanol, ethanol, isopropanol, ethyl acetate, or a combination thereof. The solution may be acidified by addition of an acid. The acid may prevent or minimize oxidative degradation of biologically-active polyphenols and other non-polyphenolic compounds in the extract. The acid may be any suitable acid, such as a mineral acid (e.g., hydrochloric acid), or an organic acid such as citric acid or acetic acid. In some embodiments, the solution comprises from 0.01% to 1% acid, such as 0.02-0.5%, 0.025-0.25%, or 0.05-0.15%. In some examples, the solution includes 0.1% hydrochloric acid.

Extraction may be performed at a temperature ranging from 0-100° C. In some embodiments, extraction is performed at a temperature ranging from 20-70° C., or at ambient temperature. Extraction may be performed for a duration ranging from several minutes to several days. To increase extraction efficiency, the plant material and solution may be mixed or agitated during extraction, such as by grinding the plant material during extraction, stirring the mixture, shaking the mixture, or homogenizing the mixture. In some embodiments, the extraction may be repeated one or more times with fresh solution to increase recovery of polyphenols and other non-polyphenolic compounds from the plant material. The liquid phases from each extraction cycle are then combined for further processing.

The liquid phase can be recovered, and the residual solids, or pulp, are discarded. Recovering the liquid phase may comprise decanting the liquid from the remaining solids and/or filtering the liquid phase to remove residual solids. The solvent (alcohol, ester, or combination thereof) can be removed from the liquid solution by any suitable means, such as evaporation (e.g., roto-evaporation), to produce an aqueous extract containing the biologically-active components in a mildly acidic solution.

In certain embodiments where the plant material includes a significant amount of oils, or lipids, an initial extraction of nonpolar components may be performed before extracting the polyphenols and other polar, non-polyphenolic compounds. Nonpolar components may be extracted by homogenizing the plant material in a nonpolar solvent, e.g., hexanes, heptanes, or a combination thereof. The solvent layer including the extracted nonpolar components is separated from the plant material and discarded.

The aqueous plant extract may be further purified by suitable means, e.g., extraction, chromatographic methods, distillation, etc., to remove non-polyphenolic compounds and/or to increase the concentration of polyphenols relative to other compounds in the extract.

The aqueous plant extract may be dried, for example by freeze-drying or other low-temperature drying methods, and ground to a powder to provide a dried plant extract. In some embodiments, the dried plant extract comprises 0.01 wt % to 25 wt % total polyphenols, such as 0.01 wt % to 10 wt %, 0.01 wt % to 5 wt %, 0.01 wt % to 2.5 wt %, 0.01 wt % to 1 wt %, 0.01 wt % to 0.5 wt %, 0.02 to 0.25 wt %, or 0.03-0.1 wt % total polyphenols. In certain embodiments, the dried plant extract further comprises non-polyphenolic compounds. For example, the dried plant extract may comprise 0.01-1 mg/g gallic acid, such as 0.05-0.5 mg/g or 0.09-0.25 mg/g gallic acid, and/or 0.001-0.1 mg/g trans-caftaric acid, such as 0.005-0.05 mg/g or 0.01-0.025 mg/g trans-caftaric acid.

The aqueous plant extract may be concentrated to a smaller volume, e.g., by evaporation, and used as an aqueous plant extract. In other embodiments, the aqueous plant extract is mixed with a carrier before drying and grinding. Suitable carriers include, for example, diatomaceous earth, silica, maltodextrin, ground grain (e.g., corn), meals (e.g., soybean or cottonseed meal) by-products (e.g., distiller's dried grains, rice hulls, wheat mill run), clays (e.g., bentonite), and combination thereof. The plant extract may be combined with a carrier in a ratio ranging from 10:1 to 1:10 by weight, such as from 5:1 to 1:5. For example, the plant extract may be mixed with diatomaceous earth in a ratio of 3:1 by weight.

Additionally, or alternatively, the additional components may comprise corn, soybean meal, wheat, wheat fiber, barley, rye, rice hulls, canola, limestone, salt, distillers dried grains with solubles (DDGS), dicalcium phosphate, sodium sesquicarbonate, methionine source, lysine source, L-threonine, biotin, folic acid, kelp, menadione dimethylpyrimidinol bisulfite, calcium aluminosilicate, or any combination thereof.

Additional information concerning feed supplement and/or additional components can be found in PCT application No. PCT/US2015/053439, and U.S. application Ser. Nos. 15/359,342, 14/699,740, 14/606,862, and 62/449,959 each of which is incorporated herein by reference in its entirety.

In some embodiments, the feed supplement does not comprise additional components. In other embodiments, the feed supplement comprises from greater than zero to 40% or more by weight additional components, such as from 0.1% to 40% by weight, or from 0.2% to 35% by weight additional components. In certain embodiments, the feed supplement comprises from 0.1% to 5% by weight additional components, such as from 0.2% to 3% by weight. In other embodiments, the feed supplement comprises from 5% to 20% by weight additional components, such as from 10% to 15% by weight. And in further embodiments, the feed supplement comprises from 20% to 40% by weight additional components, such as from 30% to 35% by weight additional components.

In some embodiments, the feed supplement comprises, consists essentially of, or consists of, silica, mineral clay, glucan, mannans, and endoglucanohydrolase; silica, mineral clay, glucan, mannans, endoglucanohydrolase, micro tracers and mineral oil; silica, mineral clay, glucan, mannans, endoglucanohydrolase, micro tracers, mineral oil, and vitamins; silica, mineral clay, glucan, mannans, endoglucanohydrolase, micro tracers, mineral oil, vitamins, and potassium sorbate; silica, mineral clay, glucan, mannans, endoglucanohydrolase, vitamins, and active yeast; silica, mineral clay, glucan, mannans, endoglucanohydrolase, micro tracers, mineral oil, and active yeast; silica, mineral clay, glucan, mannans, endoglucanohydrolase, and mineral oil; silica, mineral clay, glucan, mannans, endoglucanohydrolase, vitamins, and calcium carbonate; silica, mineral clay, glucan, mannans, endoglucanohydrolase, micro tracers, and wheat fiber; or silica, mineral clay, glucan, mannans, endoglucanohydrolase, and micro tracers. In any of these embodiments, the glucan and mannans may be provided by yeast, yeast cell wall, or yeast cell wall extract.

In some embodiments, the feed supplement does not comprise a peroxide compound. In some embodiments, the feed supplement does not comprise hydrogen peroxide. In some embodiments, the feed supplement does not comprise carbamide peroxide. In some embodiments, the feed supplement does not comprise urea. In some embodiments, the feed supplement does not comprise hydrogen peroxide and urea.

In certain embodiments, the feed supplement is a powdered supplement. In other embodiments, the feed supplement is a granulated supplement. The granulated feed supplement may comprise silica, mineral clay, glucan and/or mannans, and optionally endoglucanohydrolase as discussed above. The granulated feed supplement may have a bulk loose density of from 40 lb/ft³ to 150 lb/ft³. In some embodiments, each granule in the granular composition comprises silica, mineral clay, glucan and/or mannans, and optionally endoglucanohydrolase, in relative amounts substantially the same as a relative amount of each ingredient in the composition as whole. Each granule in the granular composition may comprise, consist essentially of, or consist of, silica, mineral clay, glucan, mannans and endoglucanohydrolase. Alternatively, or additionally, each granule may comprise a substantially homogenous blend of silica, mineral clay, glucan and mannans, and optionally endoglucanohydrolase. The composition may comprise greater than 40% by weight granules having at least one dimension between 0.149 mm (100 mesh, U.S. standard mesh size) and 4.76 mm (4 mesh), and in some embodiments, the composition comprises greater than 90% by weight granules having at least one dimension between 0.149 mm (100 mesh) and 2 mm (10 mesh). And/or the composition may comprise from greater than 0% to 100% granules by weight and from 0% to no more than 60%, such as no more than 10%, particles by weight, the granules having at least one dimension between 10 mesh (2.00 mm) and 100 mesh (0.149 mm), and the particles having at least one dimension of less than (i.e., smaller than) 100 mesh (0.149 mm). In any embodiments, the granular composition comprises plural granules, each granule comprising silica, mineral clay, glucan and mannans, the granules having a size that when administered to an animal increases expression of interleukin 10 receptor β (IL10RB) for a time period subsequent to administration, such as subsequent to the onset of administration, relative to an animal that does not receive the composition. In some embodiments the time period may be from the start of administration to from 28 days to at least 42 days. And/or the composition may have a mineral coefficient of variation of from 0% to 10%, or a proximate coefficient of variation of from 0% to 20%, or both. Additional information concerning the granular feed supplement can be found in U.S. application Ser. No. 15/878,761 which is incorporated herein by reference in its entirety.

In some embodiments, the feed supplement is administered daily to a non-ruminant animal, such as an avian, at time intervals believed or determined to be effective for achieving a beneficial result. The feed supplement may be administered in a single dose daily or in divided doses throughout the day. The amount may be from greater than zero to 500 grams per animal per day, such as from 0.5 grams to 250 grams, from 5 grams to 200 grams, or from 10 grams to 70 grams per animal per day. Alternatively, the feed supplement may be fed or administered in an amount of from greater than zero to 1000 mgs or more per kilogram of the animal's body weight per day, such as from greater than zero to 500 mgs per kilogram body weight. In other embodiments, the feed supplement is fed or administered per weight of animal feed. The feed supplement may be fed or administered in an amount of from greater than zero to 150 kg per ton (2000 pounds) of feed, such as from 0.1 kg to 100 kg per ton of feed. Alternatively, the feed supplement may be fed or administered in an amount of from greater than zero to 20 grams per kilogram of feed, such as from greater than zero to 10 grams of feed.

In some embodiments, a composition and/or combination comprises the disclosed combination and/or composition comprising, consisting essentially of, or consisting of, the Bacillus combination (for example Bacillus coagulans, Bacillus subtilis, Bacillus licheniformis and Bacillus amyloliquefaciens), the essential oil composition (for example, comprising oregano essential oil) and optionally yucca and/or quillaja, and a second composition comprising, consisting essentially of, or consisting of, one or more of silica, mineral clay, glucan, mannans, and endoglucanohydrolase.

F. Additional DFM(s)

The disclosed combination and/or composition can be administered to a non-ruminant animal, such as an avian, in combination with one or more additional DFMs. The additional DFM(s) may be any DFM suitable for administration to the particular animal. In some embodiments, the animal is a poultry, particularly a chicken or a turkey, and the additional DFM is a DFM that provides a benefit to the poultry. The additional DFM may be, by way of example and without limitation, an additional Bacillus species, Lactobacillus, Enterococcus, Bifidobacterium, Propionibacterium, Streptococcus, Pediococcus, yeast, or a combination thereof.

Exemplary additional DFMs include, but are not limited to, Bacillus alcalophilus, Bacillus alvei, Bacillus aminovorans, Bacillus aneurinolyticus, Bacillus anthracis, Bacillus aquaemaris, Bacillus atrophaeus, Bacillus boroniphilus, Bacillus brevis, Bacillus caldolyticus, Bacillus centrosporus, Bacillus cereus, Bacillus circulans, Bacillus firmus, Bacillus flavothermus, Bacillus fusiformis, Bacillus galliciensis, Bacillus globigii, Bacillus infernus, Bacillus larvae, Bacillus laterosporus, Bacillus lentus, Bacillus megaterium, Bacillus mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophilus, Bacillus thermoglucosidasius, Bacillus thuringiensis, Bacillus vulgatis, Bacillus weihenstephanensis, Lactobacillus acidophilis, Lactobacillus plantarum, Lactobacillus casei, Lactobacillus gallinarum, Lactobacillus lactis, Lactobacillus salivarius, Lactobacillus reuteri, Lactobacillus bulgaricus, Bifidobacterium pseudolongum, Bifidobacterium thermophilium, Bifidobacterium longum, Bifidobacterium lactis, Bifidobacterium animalis, Bifidobacterium bifidum, Bifidobacterium infantis, Streptococcus bovis, Streptococcus faecium, Enterococcus faecium, Enterococcus faecalis, Enterococcus diacetylactis, Saccharomyces cerevisiae, Saccharomyces boulardii Aspergillus oryzae, Aspergillus niger, Selenomonas ruminantium, Megasphaera elsdenii, Propionibacterium freudenreichii, Propionibacterium shermanii, Propionibacterium acidipropionici, Propionibacterium fensenii, Prevotella bryantii, Pediococcus acidilactici, Pediococcus cerevisiae, or a combination thereof. In certain embodiments, Bacillus pumilus may be administered in combination with the disclosed combination and/or composition.

G. Metal Chelates

Additionally, or alternatively, embodiments of the disclosed combination and/or composition may comprise a metal chelate. A metal chelate comprises at least one metal ion and at least one ligand associated with, such as binding to, the metal ion(s). In some embodiments, the ligand(s) can chelate and/or coordinate with one or more biologically-, nutritionally- and/or biocidally-relevant metals to form a metal chelate. As understood by a person of ordinary skill in the art, relevant metals can be used, for example, as part of a nutritional or biological supplement; are known to be beneficial to animals; and/or are substantially non-toxic when administered in the amounts disclosed herein. Additionally, or alternatively, the metal may have a biocidal property, and may be administered as a metal chelate.

Exemplary metals may include, but are not limited to, iron, copper, zinc, manganese, chromium, calcium, potassium, sodium, magnesium, cobalt, nickel, molybdenum, vanadium, strontium, selenium, or a combination thereof. In some disclosed embodiments, the metal is selected to provide a metal ion having a valency of +1, +2, +3, or more. For certain disclosed embodiments, the metal ion has a valency of two or three, and in particular embodiments, the metal ion is, or comprises, iron (II) or iron (III).

In particular embodiments, a combination and/or composition comprises the disclosed combination and/or composition and one or more metal chelates comprising ferric (+3) ions, particularly, ferric tyrosine, ferric citrate, ferric lactate, ferric proteinate, and/or ferric lysine.

Additionally, or alternatively, metal chelates suitable for use with the disclosed combination and/or composition include, but are not limited to, metal chelates having a formula

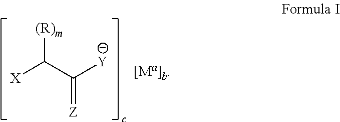

With reference to Formula 1:
m is 0, 1 or 2;
a is from 1 to 6 or more, such as from 2 or 3;
b is 1 or 2;
c is 1, 2 or 3;
X can be selected from —C(R¹)₃, OH, CO₂R¹, CO₂H, OR², NH₂, NR²H, NR²R³, —(C(R¹)₂)ₙONO₂, —(C(R¹)₂),NO₂, SH, SR² wherein each R¹, R² and R³ independently is selected from hydrogen, aliphatic, haloaliphatic, haloheteroaliphatic, heteroaliphatic, aromatic, aliphatic-aromatic, heteroaliphatic-aromatic or any combination thereof, and n is 1 to 6;
Y can be selected from NH₂, NHR³, NR³R⁴, SH, OR³, OH wherein R³ and R⁴ can independently be selected from aliphatic, haloaliphatic, haloheteroaliphatic, heteroaliphatic, aromatic, aliphatic-aromatic, heteroaliphatic-aromatic or any combination thereof;
Z can be selected from O, S, NH, NR⁵ wherein R⁵ can be selected from aliphatic, haloaliphatic, haloheteroaliphatic, heteroaliphatic, aromatic, aliphatic-aromatic, heteroaliphatic-aromatic or any combination thereof; and
each R independently is selected from halogen, aliphatic, haloaliphatic, haloheteroaliphatic, heteroaliphatic, aromatic, aliphatic-aromatic, heteroaliphatic-aromatic, or any combination thereof; and
M is a metal ion as previously described.

In some embodiments, m is 1 or 2, i.e. m is not 0. In some embodiments, when X=—C(R¹)₃, then X and one R¹ together with the atoms to which they are attached form a cyclic ring, such as an aliphatic, heteroaliphatic, aryl, or heteroaryl ring.

In some embodiments of Formula I, the ligand is an acid, such that Z is O and Y is OH. The acid may be an amino acid (X is NH₂, NR²H, or NR²R³) or a hydroxyl acid X is OH), such as an α-hydroxy acid, a β-hydroxy acid, or a γ-hydroxy acid.

In particular disclosed embodiments, the metal chelate may have a structure according to any one of the following formulas:

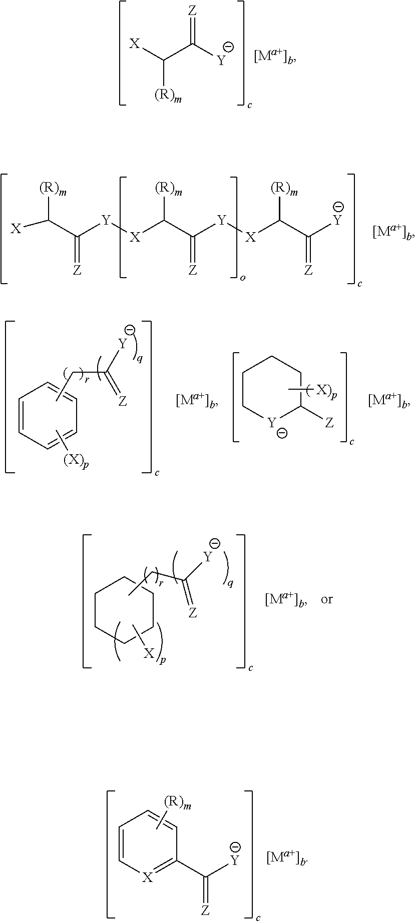

A person of ordinary skill in the art will understand that the above formulas, including Formula I, are stereoambiguous. That is, these formulas do not indicate the relative or absolute stereochemistry of the potential stereoisomers; nevertheless, all such stereoisomers are within the scope of the disclosed metal chelates.

The metal chelate may further comprise one or more counterions. The number and nature of the counterion(s) may be selected to result in a charge-neutral metal chelate. Suitable counterions include, but are not limited to, sodium, potassium, lithium, calcium, magnesium, chloride, bromide, iodine, fluoride, sulfate, carbonate, nitrate, hydroxide, or a combination thereof.

Further, in certain embodiments, a metal chelates disclosed herein may be formed using two or more different ligands. That is, an exemplary metal chelate disclosed herein may comprise a metal atom or its ion that binds with, for example, two lactic acid molecules and one oxalic acid molecule.

In some embodiments, the metal chelates disclosed herein can be metal complexes of aliphatic hydroxy acids, metal complexes of cyclic hydroxy acids (such as cyclic aliphatic hydroxy acids, aromatic hydroxy acids, etc.), metal complexes of carbohydrates, metal complexes of partially hydrolyzed or hydrolyzed proteins (such as, metal proteinates), metal complexes of amino acids, metal complexes of oligopeptides, salts and/or hydrates thereof; and any combinations thereof. In certain embodiments, the metal chelates disclosed herein can be iron (II) complexes of aliphatic hydroxy acids, iron (II) complexes of cyclic hydroxy acids (including, cyclic aliphatic hydroxy acids, aromatic hydroxy acids, etc.), iron (II) complexes of carbohydrates, iron (II) complexes of partially hydrolyzed or hydrolyzed proteins, iron (II) complexes of amino acids, iron (II) complexes of oligopeptides, or any combinations thereof. In certain embodiments, the metal chelates disclosed herein can be iron (III) complexes of aliphatic hydroxy acids, iron (III) complexes of cyclic hydroxy acids (such as, cyclic aliphatic hydroxy acids, aromatic hydroxy acids, etc.), iron (III) complexes of carbohydrates, iron (III) complexes of partially hydrolyzed or hydrolyzed proteins, iron (III) complexes of amino acids, iron (III) complexes of oligopeptides, or any combinations thereof.

In certain embodiments, metal complexes of the aliphatic hydroxy acids may include, but are not limited to, metal complexes of α-hydroxy acids, metal complexes of β-hydroxy acids, metal complexes of γ-hydroxy acids, or any combinations thereof. In particular disclosed embodiments, iron (II) complexes of the aliphatic hydroxy acids may include, but are not limited to, iron (II) complexes of α-hydroxy acids, iron (II) complexes of β-hydroxy acids, iron (II) complexes of γ-hydroxy acids, or any combinations thereof. Exemplary iron (II) complexes of α-hydroxy acids include, but are not limited to, ferrous lactate, ferrous glycolate, ferrous citrate, ferrous mandelate, ferrous tartrate, iron (II) salicylate, iron (II) p-hydroxy benzoate, ferrous complex of isoleucic acid, ferrous valate; salts and/or hydrates thereof. In particular disclosed embodiments, iron (III) complexes of the aliphatic hydroxy acids may include, but are not limited to, iron (III) complexes of α-hydroxy acids, iron (III) complexes of 3-hydroxy acids, iron (III) complexes of γ-hydroxy acids, or any combinations thereof. Exemplary iron (III) complexes of α-hydroxy acids include, but are not limited to, ferric lactate, ferric glycolate, ferric citrate, ferric mandelate, ferric tartrate, ferric complex of isoleucic acid, ferric valate; salts and/or hydrates thereof. In certain embodiments, metal complexes of cyclic hydroxy acids (i.e., iron (II) complexes of cyclic hydroxy acids) may include, but are not limited to, ferrous quinate, ferrous complex of o-hydroxy benzoic acid, ferrous complex of m-hydroxy benzoic acid, ferrous complex of p-hydroxy benzoic acid, ferrous complex of pyridine-2-carboxylic acids, or any combinations thereof. Exemplary iron (III) complexes of cyclic hydroxy acids may include, but are not limited to, ferric quinate, ferric complex of o-hydroxy benzoic acid, ferric complex of m-hydroxy benzoic acid, ferric complex of p-hydroxy benzoic acid, iron (III) γ-hydroxy butyrate, ferric β-hydroxy butyrate, iron(III) m-hydroxy benzoate, iron (III) γ-hydroxy pentanoate, iron (III) β-hydroxy pentanoate, ferric β-hydroxy propionate, iron (III) p-hydroxy benzoate, iron (III) salicylate, ferric complex of pyridine-2-carboxylic acids, or any combinations thereof. Exemplary iron (II) complex of carbohydrates may include, but are not limited to, iron (II) complex of amino sugars (e.g., D-glucosamine, etc.), iron (II) complex of monosaccharides (e.g., D-glucose, L-glucose, ribose, arabinose, xylose, lyxose, galactose, gulose, mannose, etc.), iron (II) complex of disaccharides (e.g., sucrose, lactose, etc.) or any combinations thereof. Exemplary iron (III) complex of carbohydrates may include, but are not limited to, iron (III) complex of amino sugars (e.g., D-glucosamine, etc.), iron (III) complex of monosaccharides (e.g., D-glucose, L-glucose, ribose, arabinose, xylose, lyxose, galactose, gulose, mannose, etc.), iron (III) complex of disaccharides (e.g., sucrose, lactose, etc.) or any combinations thereof.

Exemplary iron (II) complex of amino acids may include, but are not limited to, iron (II) complex of alanine, iron (II) complex of arginine, iron (II) complex of asparagine, iron (II) complex of aspartic acid, iron (II) complex of cysteine, iron (II) complex of glutamine, iron (II) complex of glutamic acid, iron (II) complex of glycine, iron (II) complex of histidine, iron (II) complex of isoleucine, iron (II) complex of leucine, iron (II) complex of lysine, iron (II) complex of methionine, iron (II) complex of phenylalanine, iron (II) complex of proline, iron (II) complex of serine, iron (II) complex of threonine, iron (II) complex of tryptophan, iron (II) complex of tyrosine, iron (II) complex of valine, iron (II) complex of selenocysteine and iron (II) complex of pyrrolysine. In some embodiments, the iron (II) complex is not ferrous sulfate and tyrosine to form in-vivo ferrous-tyrosine complex. In some embodiments, the iron (II) complex is not ferrous sulfate and L-DOPA to form in-vivo ferrous-L-DOPA complex. In some embodiments, the iron (II) complex is not ferrous sulfate and L-phenylalanine to form in-vivo ferrous-L-phenylalanine complex. In some embodiments, the iron (II) complex is not ferrous sulfate and quinic acid to form in-vivo ferrous-quinate complex.

Exemplary iron (III) complex of amino acids may include, but are not limited to, iron (III) complex of alanine, iron (III) complex of arginine, iron (III) complex of asparagine, iron (III) complex of aspartic acid, iron (III) complex of cysteine, iron (III) complex of glutamine, iron (III) complex of glutamic acid, iron (III) complex of glycine, iron (III) complex of histidine, iron (III) complex of isoleucine, iron (III) complex of leucine, iron (III) complex of lysine, iron (III) complex of methionine, iron (III) complex of phenylalanine, iron (III) complex of proline, iron (III) complex of serine, iron (III) complex of threonine, iron (III) complex of tryptophan, iron (III) complex of tyrosine, iron (III) complex of valine, iron (III) complex of selenocysteine, and iron (III) complex of pyrrolysine. Although in some embodiments, the disclosed iron (II)/amino acid complexes, or iron (III)/amino acid complexes of the present disclosure comprise L-isoform of the amino acid moieties, D-isoform amino acid moieties, or a combination of both D- and L-isoforms.

In some embodiments, the metal-chelated peptides disclosed herein may be, or may include, metal-chelated oligopeptides which include two or more amino acids linked in a chain, where the carboxylic acid group of one amino acid and the amino group of another amino acid together form a peptide (—OC—NH—) bond. In some embodiments, the metal-chelated oligopeptides disclosed herein may comprise from two amino acids to twenty amino acids. In certain embodiments, the metal-chelated oligopeptides may include, but are not limited to metal-chelated dipeptides, metal-chelated tripeptides, metal-chelated tetrapeptides, metal-chelated pentapeptides, metal-chelated hexapeptides, metal-chelated heptapeptides, metal-chelated octapeptides, metal-chelated nonapeptides, metal-chelated decapeptides, or any combinations thereof. In particular disclosed embodiments, the iron (II)-chelated oligopeptides may include, or may be, iron (II)-chelated dipeptides, iron (II)-chelated tripeptides, iron (II)-chelated tetrapeptides, iron (II)-chelated pentapeptides, iron (II)-chelated hexapeptides, iron (II)-chelated heptapeptides, iron (II)-chelated octapeptides, or any combinations thereof. Exemplary iron (II)-chelated peptides may include, but are not limited to, iron (II)-chelated Gly-Gly, Gly-Leu, iron (II)-chelated Ala-Phe, iron (II)-chelated Phe-Ile-Val, iron (II)-chelated Leu-Pro-Trp, iron (II)-chelated Pro-Leu-Gly, iron (II)-chelated Gly-Gly-Gly, iron (II)-chelated Gly-Lys-Val-Ser, iron (II)-chelated Met-Thr-Cys-Gln, iron (II)-chelated Lys-Gly-Arg-Trp-Phe, iron (II)-chelated Ala-Leu-Pro-Gly-Ala, iron (II)-chelated Gly-Phe-Arg-His-Gly-Gly, iron (II)-chelated Ala-Phe-Phe-Ile-Val-Gly-Gly, iron (II)-chelated Gly-Lys-Val-Ser-Pro-Leu-Gly-Pro.

In particular disclosed embodiments, the iron (III)-chelated oligopeptides may include, or may be, iron (III)-chelated dipeptides, iron (III)-chelated tripeptides, iron (III)-chelated tetrapeptides, iron (III)-chelated pentapeptides, iron (III)-chelated hexapeptides, iron (III)-chelated heptapeptides, iron (III)-chelated octapeptides, or any combinations thereof. Exemplary iron (III)-chelated peptides may include, but are not limited to, iron (III)-chelated Gly-Gly, Gly-Leu, iron (III)-chelated Ala-Phe, iron (III)-chelated Phe-Ile-Val, iron (III)-chelated Leu-Pro-Trp, iron (III)-chelated Pro-Leu-Gly, iron (III)-chelated Gly-Gly-Gly, iron (III)-chelated Gly-Lys-Val-Ser, iron (III)-chelated Met-Thr-Cys-Gln, iron (III)-chelated Lys-Gly-Arg-Trp-Phe, iron (III)-chelated Ala-Leu-Pro-Gly-Ala, iron (III)-chelated Gly-Phe-Arg-His-Gly-Gly, iron (III)-chelated Ala-Phe-Phe-Ile-Val-Gly-Gly, iron (III)-chelated Gly-Lys-Val-Ser-Pro-Leu-Gly-Pro.

The disclosed combination and/or composition may comprise a sufficient amount of metal chelate such that administration of the combination and/or composition to an animal provides the animal with a desired amount of metal chelate. The desired amount of metal chelate may be any effective dose as understood by a person of ordinary skill in the art. For example, the desired amount of metal chelate an amount effective as a food supplement or an amount effective as a biocidal agent. By way of example, the metal chelate may be administered to an animal, such as a human or non-human animal, such that the animal ingests and/or absorbs a total amount of the metal chelate (or an equivalent number of moles of the metal chelate) from 1 mg to 200 g per kg of the average body weight of the animal, such as, 5 mg to 150 g, 10 mg to 100 g, 50 mg to 50 g, 100 mg to 10 g, 500 mg to 50 g, or 1 g to 5 g. Exemplary amount includes, but is not limited to, 1 mg, 2 mg, 3 mg, 4 mg, 5 mg, 10 mg, 50 mg, 100 mg, 500 mg, 1 g, 5 g, 10 g, 50 g, 100 g, 150 g, or 200 g per kg of the average body weight of the animal. Additionally, or alternatively, combination and/or composition comprising the metal chelate may be administered with feed in an amount sufficient to provide from 0.001 to 20 g of the metal chelate per kg of feed, such as 0.002 to 15 g/kg, or at least 0.002 g/kg, 0.005 g/kg, 0.01 g/kg, 0.02 g/kg, 0.05 g/kg, 0.1 g/kg, 0.5 g/kg, 1 g/kg, 2 g/kg, 5 g/kg, 10 g/kg, 15 g/kg of the feed.

H. Chromium Compound

In certain embodiments, the disclosed combination and/or composition is administered in combination with one or more chromium compounds. Chromium compounds that can be used with the disclosed combination and/or composition include any chromium compound suitable for feed, food, pharmaceutical or veterinary use. Without being bound to a particular theory, chromium may help facilitate glucose intake in cells, and therefore may provide a substantial benefit when used in combination with the disclosed combination and/or composition. In some embodiments, the chromium compound(s) comprise a chromium (III) compound. Exemplary chromium compounds include, but are not limited to, chromium organic acid compounds, such as chromium picolinate, chromium tripicolinate, chromium nicotinate, chromium polynicotinate, chromium acetate, or chromium propionate, or chromium amino acid compounds, such as chromium histidinate, chromium nicotinate-glycinate, chromium glycinate, chromium aspartate, chromium methionine, chromium trimethionine, or chromium phenylalanine; chromium halides, such as chromium chloride, chromium bromide, chromium iodine or chromium fluoride; chromium yeast; chromium carbonate; chromium nitrate; chromium sulfate; chromium phosphate; chromium nitrite; or a combination thereof. Additional information concerning chromium compounds can be found in U.S. Patent Publication No. 2010/0178362, which is incorporated herein by reference. The amount of chromium compound may be sufficient to provide a daily dose of from 0.001 milligram to 5000 milligrams of a total chromium compound per kilogram body weight, such as from 0.01 milligram total chromium compound to 1000 milligrams per kilogram body weight, from 0.1 milligram to 100 milligrams per kilogram body weight, from 0.5 milligram to 25 milligrams per kilogram body weight, or from 1 milligram to 10 milligrams per kilogram body weight.

In some embodiments, the amount of chromium compound in the combination is selected to provide a sufficient amount of chromium to the subject. The sufficient amount of chromium may be from 0.5 μg per day to 10,000 μg per day or more, such as from 5 μg to 10,000 μg/day, from 25 μg to 10,000 μg per day, from 50 μg to 10,000 μg per day, from 100 μg to 10,000 μg per day, from 200 μg to 10,000 μg per day, from 300 μg to 10,000 μg per day, from 400 μg to 10,000 μg per day, from 500 μg to 10,000 μg per day, from 750 μg to 10,000 μg per day, from 1,000 μg to 10,000 μg per day, from 1500 μg to 10,000 μg per day, from 2,000 μg to 10,000 μg per day, from 2500 μg to 10,000 μg per day, from 3000 μg to 10,000 μg per day, from 3500 μg to 10,000 μg per day, from 4000 μg to 10,000 μg per day, from 4500 μg to 10,000 μg per day, from 5,000 μg to 10,000 μg per day, from 6000 μg to 10,000 μg per day, from 7000 μg to 10,000 μg per day, from 8000 μg to 10,000 μg per day, from 9000 μg to 10,000 μg per day or more chromium/day.

I. Allicin, alliin and/or alliinase

Additionally, or alternatively, the disclosed combination and/or composition can be administered in combination with allicin, alliin, alliinase, or any combination thereof. Allicin (diallyl thiosulfate; 2-Propene-1-sulfinothioic acid S-2-propenyl ester) is a compound found in garlic, such as raw garlic.

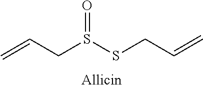

When extracted, it may be an oily, yellowish liquid. Allicin may have medicinal and/or health benefits when consumed by animals. Benefits of allicin include, but are not limited to, an immunity booster; a blood thinner; an anti-oxidant; an anti-bacterial agent, such as against E. coli; an anti-inflammatory; an anti-viral; an anti-fungal; or may alleviate symptoms of bacterial, viral or fungal infections. Allicin is typically produced from alliin ((2R)-2-amino-3-[(S)-prop-2-enylsulfinyl]propanoic acid) in damaged garlic cells by the action of the enzyme alliinase.

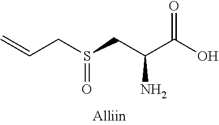

When the garlic cells are damaged, such as by chopping, crushing, or cooking the garlic, the alliinase enzyme converts the alliin into allicin. Allicin, alliin, and/or alliinase may be provided as whole garlic cloves or bulbs; crushed, mashed, or chopped garlic; a garlic extract; and/or as a synthesized or isolated compound.

J. Yeast

Additionally, or alternatively, the disclosed combination and/or composition can be administered in combination with a microorganism, such as yeast. The yeast may be a yeast culture, a live yeast, a dead yeast, yeast extract, or a combination thereof. The yeast may be a baker's yeast, a brewer's yeast, a distiller's yeast, a probiotic yeast or a combination thereof. Exemplary yeast's include, but are not limited to, Saccharomyces cerevisiae, Saccharomyces boulardii, Saccharomyces pastorianus, Brettanomyces bruxellensis, Brettanomyces anomalus, Brettanomyces custersianus, Brettanomyces naardenensis, and Brettanomyces nanus, Candida stellata, Schizosaccharomyces pombe, Torulaspora delbrueckii, or Zygosaccharomyces baiii.

K. Growth Factor

Additionally, or alternatively, the disclosed combination and/or composition can be administered in combination with a growth factor, including, but not limited to, an insulin-like growth factor (IGF), or a transforming growth factor (TGF). Growth factors belong to a complex family of biological compounds, such as peptide hormones, that include transforming growth factors, insulin-like growth factors, epithelial growth factors, and placental growth factors. Growth factors may be useful for therapeutic applications and/or as feed supplements.

Insulin-like growth factors (IGFs) are polypeptides that have a high sequence similarity to insulin. IGFs are part of a system that enables cells to communicate with their environment. IGFs are useful for regulation of normal physiology and have a role in cell proliferation and inhibition of cell death. IGFs can be obtained from biological sources, such as milk or blood, by methods known to persons of ordinary skill in the art. The methods include adding anti-coagulants to whole blood, centrifuging and separating the plasma. Alternatively, IGFs can be extracted from milk, such as by chromatography, including cation exchange chromatography, as described in European patent No. EP 0 313 515, incorporated herein by reference. IGF also can be produced by recombinant techniques, such as by using yeast, as in the methods described in U.S. Pat. Nos. 6,117,983, 7,071,313 and 7,193,042, or bacteria, as described in U.S. Pat. Nos. 5,084,384, 5,489,517, and 5,958,754, all of which are incorporated herein by reference.

Insulin-like growth factor-1 (IGF-1 or IGF-I) is typically secreted by the liver and is important for achieving maximal growth, such as childhood growth, but also continues to have an effect in adults. Insulin-like growth factor-2 (IGF-2 or IGF-II) is thought to be a major growth factor for fetal growth and early development. As such, it is typically important during gestation.

Transforming growth factors (TGFs), such as transforming growth factor beta (TGF-beta), are polypeptides that are important for several functions within a cell, such as proliferation and cellular differentiation. They also may have a role in immunity and wound healing.

Although growth factors, such as IGFs and TGFs often are obtained from animal sources, such as milk or blood, they are usually bound to a binding protein that causes the IGF to be inactive. An IGF bound to a binding protein is referred to as an inactive IGF. For example, it is estimated that more than 99% of IGF-1 in plasma is bound to a binding protein. To form an active IGF, the binding protein is separated from the IGF polypeptide. During activation, the amount of IGF typically remains the same, but the ratio of active IGF to inactive IGF increases. Methods to form active IGF from inactive IGF are known in the art. For example, methods for increasing the ratio of active IGF to inactive IGF include processes routinely used to activate functional proteins obtained from a biological material. Such processes include, but are not limited to, exposing the biological material to heat shock, temperature adjustment, alcohol extraction, pH adjustment, enzyme addition, ionic changes, other chemical additions, and pressure, or combinations thereof. Without being bound to a particular theory, such methods typically cause the dissociation of the binding protein from the IGF protein. Also, methods for measuring the concentration of active IGF are known to persons of ordinary skill in the art. For example, suitable assays are commercially available, including solid phase sandwich ELISA assays that specifically measure IGF that is not bound to a binding protein (e.g., R&D Systems, catalog number DFG100).

### V. Beneficial Results from Administering the Bacillus Combination

Administering the disclosed combination and/or composition to a non-ruminant animal, such as an avian, particularly poultry, has provided a substantial beneficial result when compared to administering each of the respective components individually. These beneficial results are determined by considering, for example, feed conversion rate, average body weight, average body weight gain, body weight coefficient of variation, breast meat yield, bird mortality, lesion scores, necrotic enteritis incidence, Salmonella/E. Coli/Clostridium perfringens (CP) incidence, and/or oocysts in fecal matter at various times during chick rearing. With reference to Example 1, these benefits can be determined by comparing feed conversion rate (FCR) for birds fed a basal diet and challenged with CP relative to birds that receive the disclosed combination and/or composition.

Administration of the disclosed combination and/or composition to poultry may reduce E. coli in the poultry. The amount of E. coli reduction may be from greater than zero to 25% or more, such as a reduction of from 5% to 25%, or 10% to 22%, compared to an amount of E. coli present in poultry that are not administered the combination. The reduction may be identified at various times, such as at 21 and/or 42 days old for certain disclosed working embodiments.

Administration of the disclosed combination and/or composition to poultry may reduce Aerobic Plate Count (APC) in the poultry. The APC reduction may be from greater than zero to 20% or more, such as a reduction of from 5% to 20%, or 10% to 18%, compared to an amount of APC present in poultry that are not administered the combination. The reduction may be identified at various times, such as at 21 and/or 42 days old for certain disclosed working embodiments.

Administration of the disclosed combination and/or composition to poultry may reduce Salmonella in the poultry. The Salmonella reduction may be from greater than zero to 65% or more, such as a reduction of from 25% to 65%, 35% to 65%, or from 45 to 65%, compared to an amount of Salmonella present in poultry that are not administered the combination. The reduction may be identified at various times, such as at 21 and/or 42 days old for certain disclosed working embodiments.

Administration of the disclosed combination and/or composition to poultry may reduce Clostridium perfringens in the poultry. The Clostridium perfringens reduction may be from greater than zero to 30% or more, such as a reduction of from 5% to 30%, 10% to 30%, or from 15% to 30%, compared to an amount of Clostridium perfringens present in poultry that are not administered the combination. The reduction may be identified at various times, such as at 21 and/or 42 days old for certain disclosed working embodiments.

Administration of the disclosed combination and/or composition to poultry may reduce fecal oocysts in the poultry. The oocysts reduction may be from greater than zero to 90% or more, such as a reduction of from 50% to 90%, or 75% to 90%, compared to an amount of oocysts present in poultry that are not administered the combination. The reduction may be identified at various times, such as at 21 and/or 42 days old for certain disclosed working embodiments.

Administration of the disclosed combination and/or composition to poultry may result in an improved lesion score in the poultry. The lesion score may be improved (i.e. lowered) by from greater than zero to 75% or more, such as from 25% to 75%, or from 30% to 75%, compared to a lesion score of poultry that are not administered the combination. The improvement may be identified at various times, such as at 21 and/or 42 days old for certain disclosed working embodiments.

Administration of the disclosed combination and/or composition to poultry may result in an improved feed conversion rate in the poultry. The feed conversion rate may be improved (i.e. lowered) by from greater than zero to 10% or more, such as from 2% to 8%, or from 4% to 8%, compared to a feed conversion rate of poultry that are not administered the combination. The improvement may be identified at various times, such as poultry at 14, 21 and/or 42 days old.

Administration of the disclosed combination and/or composition to poultry may result in a reduced poultry mortality rate. The mortality rate may be reduced by from greater than zero to 95% or more, such as from 50% to 95%, from 75% to 95% or from 80% to 95%, compared to a mortality rate of poultry that are not administered the combination. The improvement may be identified at various times, such as when the poultry are 14, 21 and/or 42 days old.

Administration of the disclosed combination and/or composition to poultry may result in an improved average weight gain. The weight gain may be increased by from greater than zero to 20% or more, such as from 5% to 15%, from 7% to 15% or from 10% to 15%, compared to an average weight gain of poultry that are not administered the combination. The improvement may be identified at various times, such as when the poultry are 14, 21 and/or 42 days old.

### VI. Examples

The following examples are provided to illustrate certain features of exemplary working embodiments. A person of ordinary skill in the art will appreciate that the scope of the invention is not limited to the particular features of these examples.

### Example 1

### Study Overview

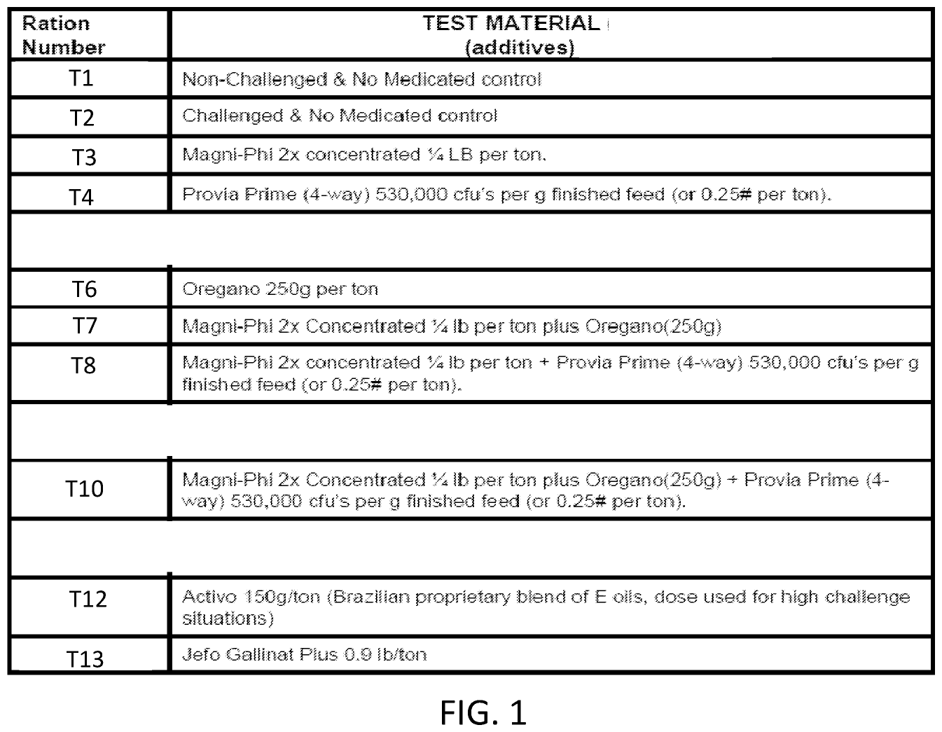

The test period began on Trial Day 0 (day of hatch of chicks, which were fed a commercial-type mash feed, and ended on Trial Day 42. Each test group or experimental unit contained 52 mixed-sex broilers (50:50 ratio) randomly assigned into 12 replicates per group for a total number of 624 animals per treatment on study. FIG. 1 provides the treatment groups. With respect to FIG. 1, “Magni-Phi 2× concentrated” refers to a combination of Yucca schidigera and Quillaja saponaria, and/or extracts thereof, in a ratio of 90% to 95%, such as 92% to 93%, Quillaja saponaria and/or extracts thereof, to 5% to 10%, such as 7% to 8%, Yucca schidigera and/or extracts thereof. Provia Prime™ refers to a combination of Bacillus subtilis, Bacillus licheniformis, Bacillus amyloliquefaciens and Bacillus coagulans as disclosed herein. And “Oregano” refers to oregano essential oil as described herein.

Chicks were randomly assigned to treatments on Trial Day 0 (or at hatch) and were not replaced during the course of the trial. The chicks were observed daily for signs of unusual grow-out patterns or health problems. Body weights, food consumption and feed conversion were measured on Trial Days 14, 21, 35 and 42. Lesion scores were determined at 21 and 42 days of age. All birds in all rations received a Coccidiosis Vaccine, as normally administered by the Hatchery source. Feedgrade and water-medicated antibiotics were not administered during the entire trial. All birds were stressed by administering Clostridium and coccidia oocysts, along with other natural bacteria from build-up litter from a farm experiencing high mortality.

A trace mineral premix, containing all essential minor minerals, was added at the rate of 1.5 pounds per ton of complete feed. At the time of complete feed blending, the various poultry-related product sources were added to the complete feed. All rations were in mash form.

Feed was fed on a fill-fed basis throughout the entire trial test period (Days 0 to Day 42). A mash feed was fed from 0-42 days of age.

### Feed Formulation Considerations

The basal ration as shown below was used to poultry-related product test sources. A minimum of 0.5% soybean oil was added to the ration. And three ration types (during test period) were prepared:

| Feed Type | Approximate Age (days of age) | Trial Days Range |
|---|---|---|
| Starter | 0 to 21 days | Trail Day 0-21 |
| Grower | 22 to 35 days | Trial Day 22-35 |
| Finisher | 36 to 42 days | Trial Day 36-42 |

### Bacteria and Coccidial Presence in Built-Up Litter

The birds were grown on built-up litter sources that contained Clostridium perfringens and other pathogenic bacteria, in a ‘coccidial-challenge model”. Layout time among flocks was <11 days. Each pen was top-dressed a minimum of 5-million total oocysts (or 100,000-150,000 per bird)+sterile water and not incorporated into the litter. Coccidia oocysts were predominate E. cervuline and E. maxima. Each pen was “walked with plastic boot covers designed to pick up litter” at a minimum of 3-times per day, for the purpose of spreading the litter from pen-to-pen and equalize the challenge.

### Observations, Tests and Measurements

CLINICAL OBSERVATIONS: Chicks were observed at least two times daily beginning on trial day 0 to determine mortality or the onset, severity, and duration of any behavioral changes or evidence of toxicity (including fecal material condition, presence of diarrhea, nervousness, accessibility to water and feed, general bird appearance, and any adverse conditions which could have affected performance).
HEALTH EXAMS: Health exams were conducted at 21 and 42 days of age (depending upon weight gains).
MEAN BODY WEIGHTS: Body weights were taken by weighing individual chicks in a pen and recorded for Trial Days 21 and 42 (depending upon weight gains). Body weight gain was calculated by determining actual body weight gain (ending minus beginning weights) during the periods of trial days 0-21, 22-42 and 0-42. Body weights were taken on both moribund birds and test animals that were found dead during the study.
BODY WEIGHT UNIFORMITY: Body weight uniformity (CV or Coefficient of Variation) was determined on Trial Days 21 and 42.
FEED CONSUMPTION: Feed weigh-backs were taken on Trial Days 0-21, 22-42 and 0-42. Food consumption was evaluated for each pen on trial days 0-21, 22-42 and 0-42. A separate container (feed trough) was assigned for each pen. The initial tare weight for each feed barrel were recorded on Trial Day 0. Feed was added and the weight recorded. When adding feed, the feed barrel was weighed and the weight recorded (Weight out). Feed out means feed that is removed and taken out of the calculations. New feed was added and the weight recorded (Weight in). Any feed spilled was weighed then discarded.
FEED CONVERSION (weight: gain ratio): Feed Conversion was determined on Trial Days 0-21, 22-42 and 0-42.
MORTALITY: Mortality was taken daily and reported as percentage per time period for Trial Days 0-21, 22-42 and 0-42.
WATER CONSUMPTION: Water consumption was measured continuously during the course of the study.
INTESTINAL BACTERIA COUNTS: Intestinal Bacteria Counts including Clostridium perfringens, E. coli and APC (Aerobic Plate Counts) were taken at both 21 (2M and 2F from each pen) and 42 days of age (5M and 5F) from each pen.

Salmonella spp. (wild stain) Incidence per g GIT feces was determined at both 21 (2M and 2F from each pen) and 42 days of age (5M and 5F) from each pen.

Campylobacter spp. (wild stain) Incidence per g GIT feces was determined at both 21 (2M and 2F from each pen) and 42 days of age (5M and 5F) from each pen.

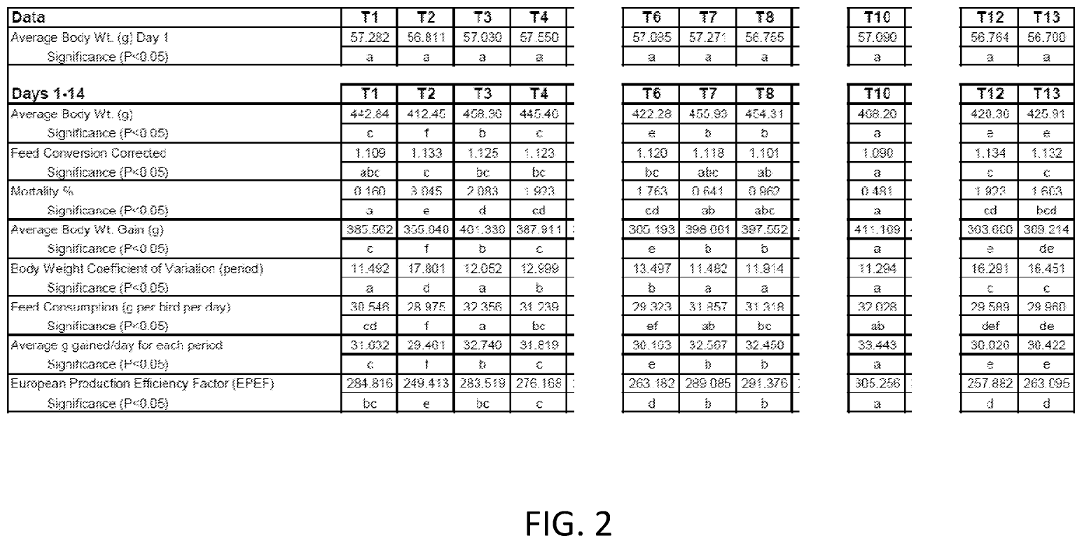

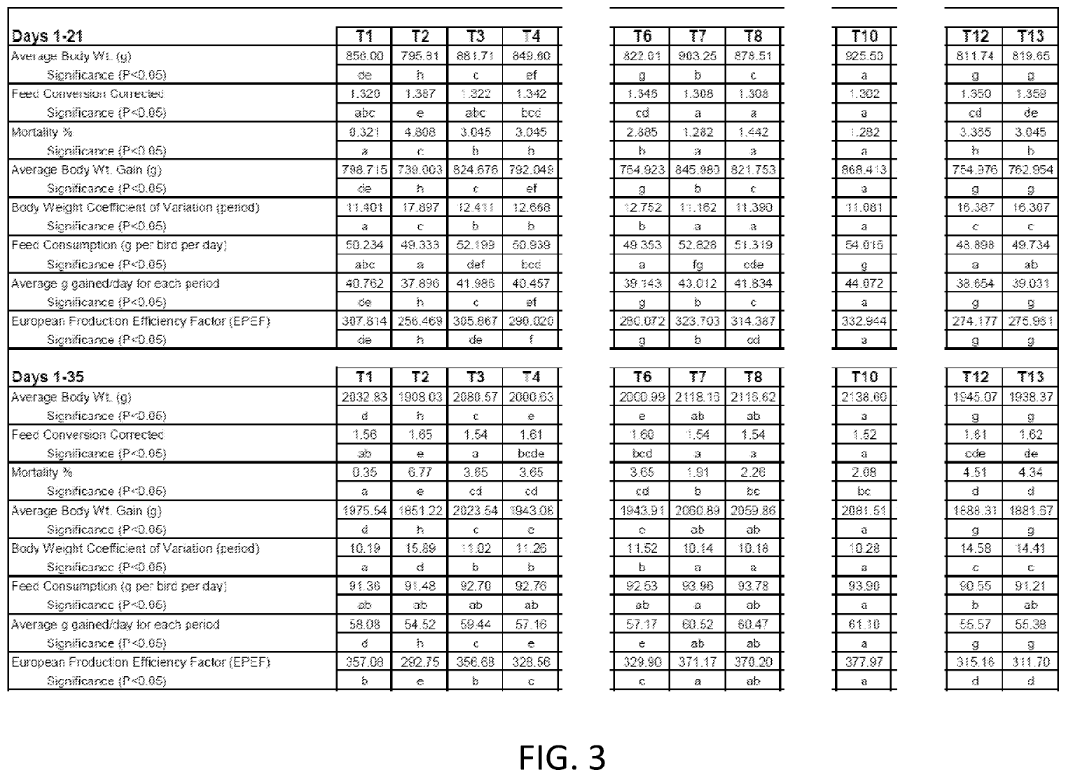

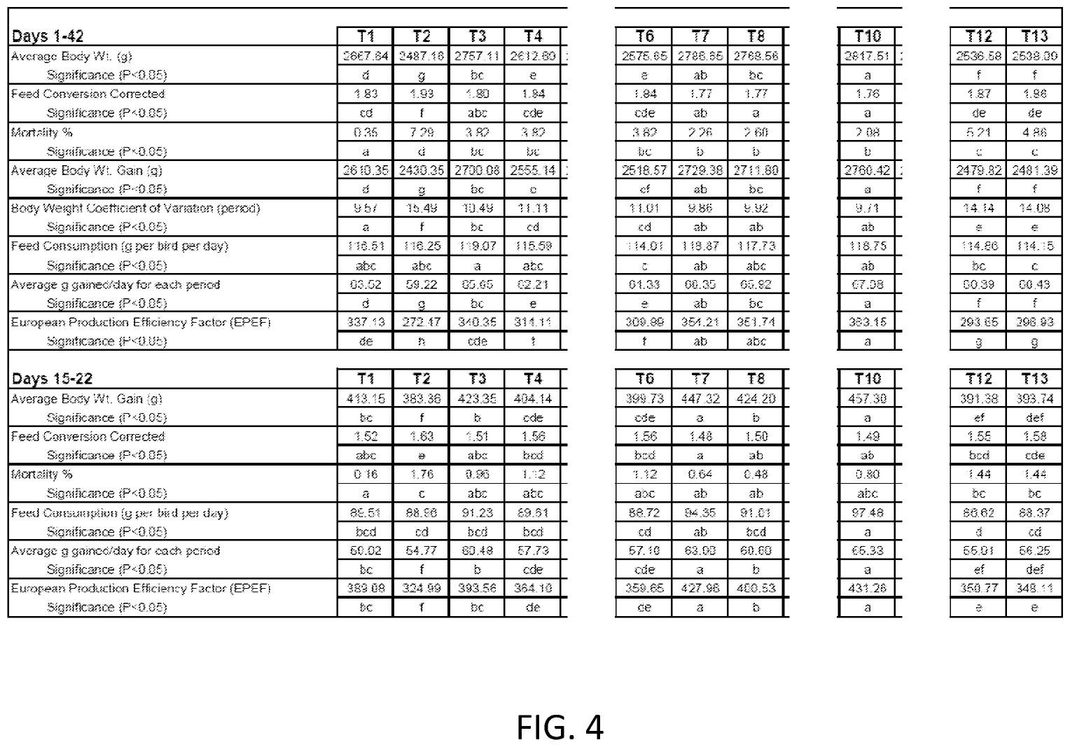

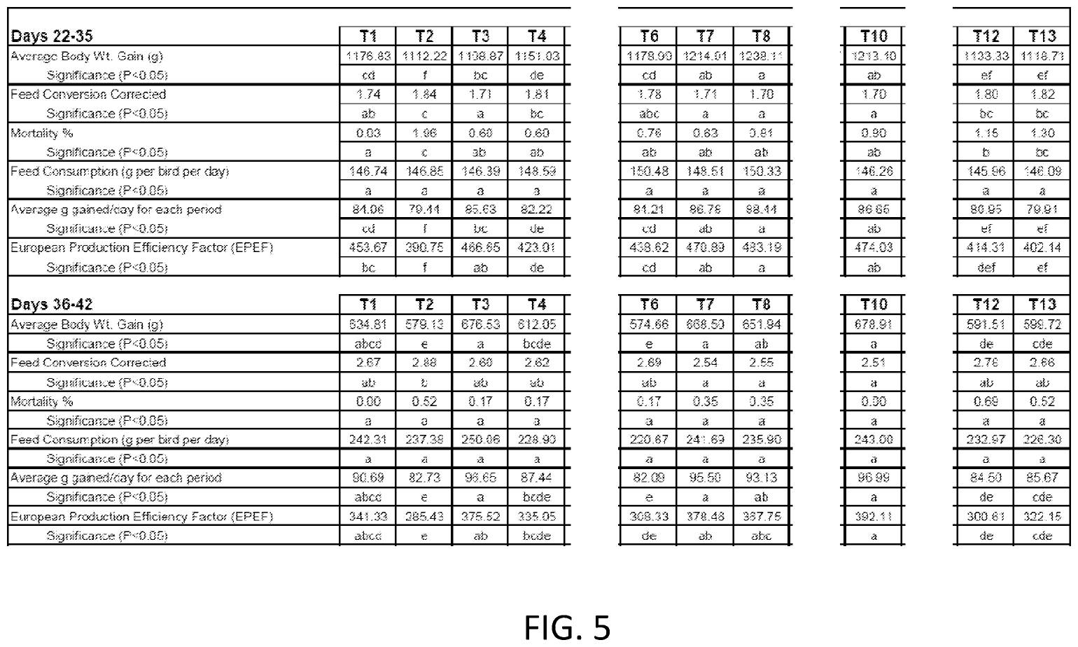

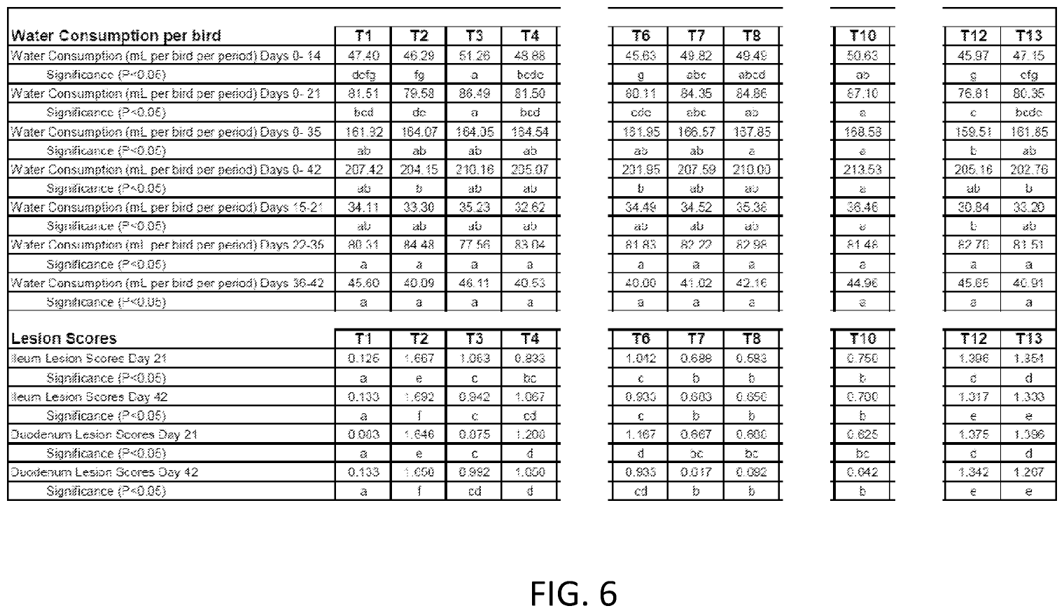

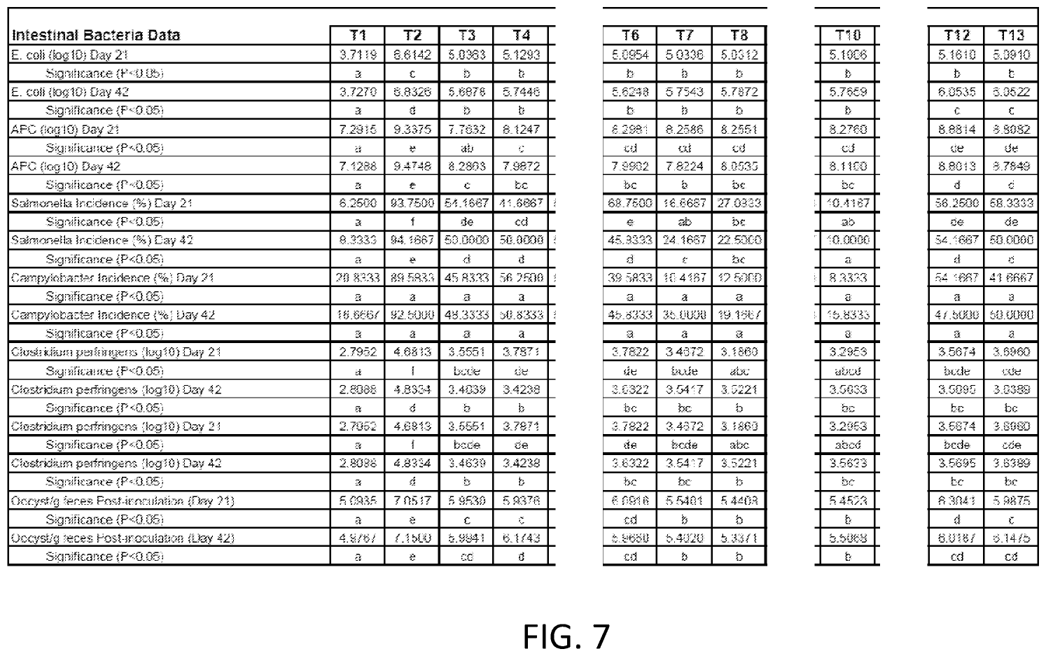

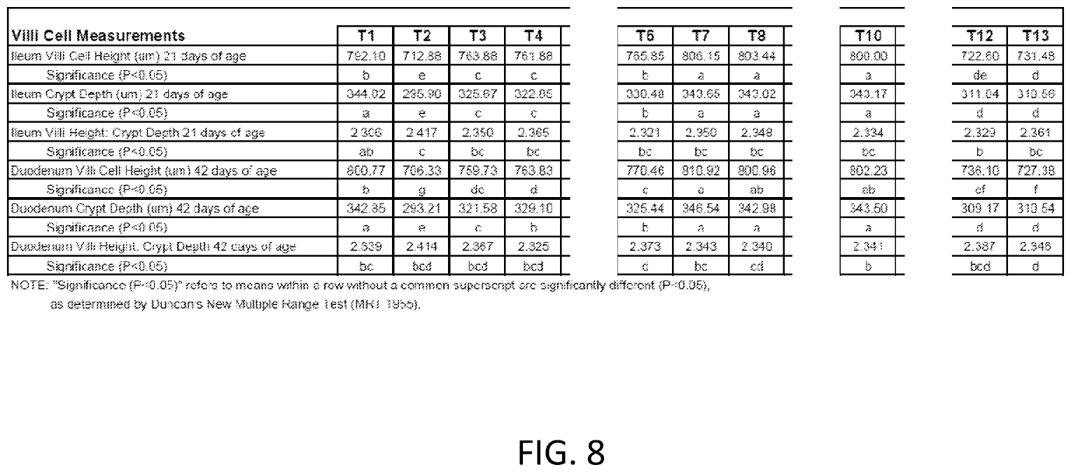

Tissue sample collection occurred on Trial Days 7, 14, 21, and 35. Intestinal samples (from 2M and 2F birds) were taken from two gut areas per bird (one at the distal to the end of the duodenal loop or 1″ to 2″ below the end of the loop and the other sample in the second in the mid gut area or a few inches anterior to Meckel's diverticulum). Both villi height and crypt depth were determined on these two areas of the gut. FIGS. 2-8 provide the data from the trial.

### Example 2

This example concerns the results of a field study in poultry, typically chickens, to compare the efficacy of the disclosed combination and/or composition against various levels of disease challenge. At least one of the test treatments comprises:
a Bacillus combination, comprising Bacillus amyloliquefaciens, Bacillus subtilis, Bacillus licheniformis and Bacillus coagulans in an amount sufficient to provide about 1M CFU of Bacillus;
a composition comprising yucca and quillaja, particularly Yucca schidigera, and Quillaja saponaria or extracts thereof in an amount sufficient to provide 350 ppm yucca and quillaja; and
an essential oil composition comprising oregano in an amount sufficient to provide 50 ppm oregano.

The study monitors parameters similar to those monitored in Example 1, for example, feed conversion, body weight and weight gain, mortality, and pathogen levels, such as, but not limited to, salmonella, E. coli, coccidia, and/or Clostridium perfringens.

The study is expected to demonstrate that the disclosed combination and/or composition provides an improved feed conversion rate and/or increased weight gain, compared to birds that are not administered the combination and/or composition, and compared to birds that are only administered one or two of the components of the disclosed combination and/or composition.

Additionally, the results will demonstrate that birds that are administered the disclosed combination and/or composition have reduced mortality and/or reduced pathogen levels when challenged, compared to birds that are not administered the combination and/or composition, and compared to birds that are only administered one or two of the components of the disclosed combination and/or composition.

### Example 3

### Study Overview

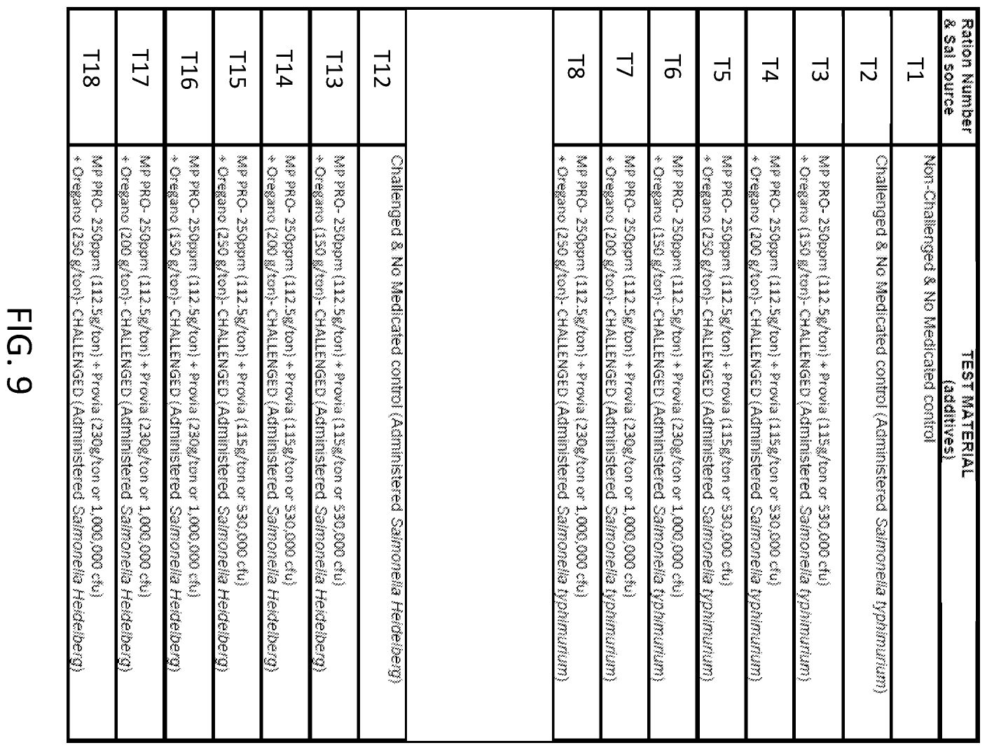

The test period began on Trial Day 0 (day of hatch of chicks, which were fed a commercial-type mash feed and end on Trial Day 42. Each test group or experimental unit were contained 52 mixed-sex broilers (50:50 ratio) randomly assigned into 12 replicates per group for a total number of 624 animals per treatment on study. FIG. 9 provides the treatment groups. With respect to FIG. 1, “MIP Pro™” refers to a combination of Yucca schidigera and Quillaja saponaria, and/or extracts thereof, in a ratio of 90% to 95%, such as 92% to 93%, Quillaja saponaria and/or extracts thereof, to 5% to 10%, such as 7% to 8%, Yucca schidigera and/or extracts thereof, with “Provia™” and Oregano. “Provia™” refers to a combination of Bacillus subtilis, Bacillus licheniformis, Bacillus amyloliquefaciens and Bacillus coagulans as disclosed herein, administered as Provia Prime™, available from Phibro Animal Health Corporation. And “Oregano” refers to oregano essential oil as described herein.

Chicks were randomly assigned to treatments of Trial Day 0 (or at hatch) and were not replaced during the course of the trial. The chicks were observed daily for signs of unusual grow-out patterns or health problems. Body weights, food consumption and feed conversion were measured on Trial Days 14, 21, 35 and 42. Lesion scores were determined at 21 and 42 days of age. All birds in all rations received Coccidiosis Vaccine, as normally administered by the Hatchery source. Feedgrade antibiotics were not administered during the entire trial. All birds were stressed by administering Clostridium and coccidia oocysts, along with other natural bacteria from build-up litter from a farm experiencing high mortality.

### Feed Formulation Considerations

The basal ration as shown below was used to poultry-related product test sources. A minimum of 0.5% soybean oil was added to the ration. And three ration types (during test period) were prepared:

| Feed Type | Approximate Age (days of age) | Trial Days Range |
|---|---|---|
| Starter | 0 to 21 days | Trail Day 0-21 |
| Grower | 22 to 35 days | Trial Day 22-35 |
| Finisher | 36 to 42 days | Trial Day 36-42 |

### Bacteria and Coccidial Presence in Built-Up Litter

The birds were grown on built-up litter sources that contained Clostridium perfringens and other pathogenic bacteria, in a ‘coccidial-challenge model”. Layout time among flocks was <11 days. Each pen was top-dressed a minimum of 5-million total oocysts (or 100,000-150,000 per bird)+sterile water and not incorporated into the litter. Coccidia oocysts were predominate E. cervuline and E. maxima. Each pen was “walked with plastic boot covers designed to pick up litter” at a minimum of 3-times per day, for the purpose of spreading the litter from pen-to-pen and equalize the challenge.

### Observations, Tests and Measurements

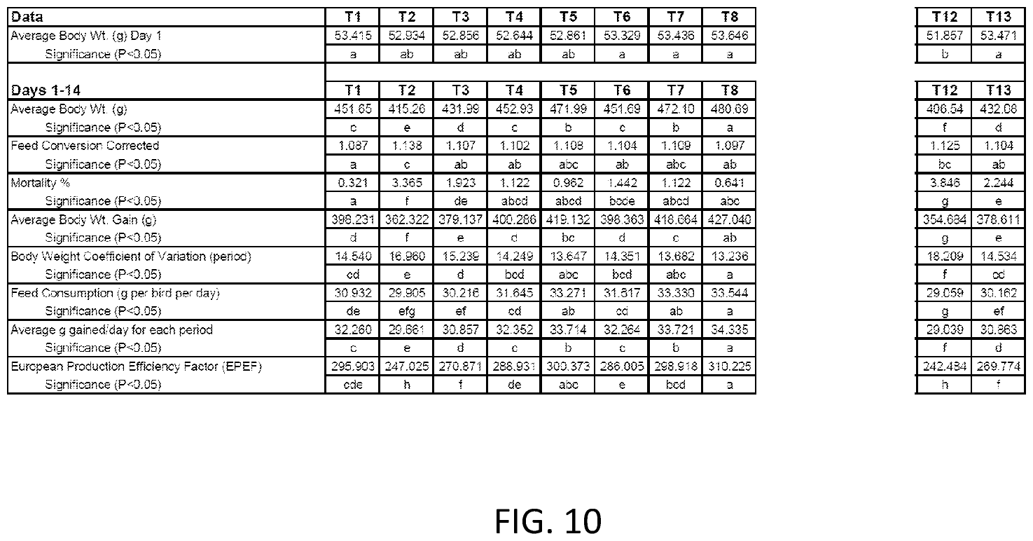

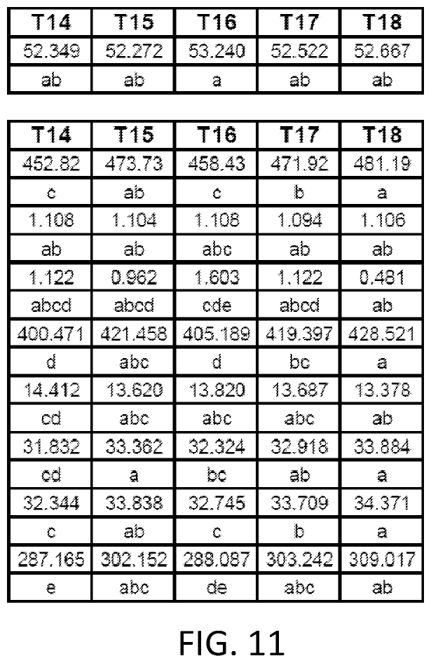

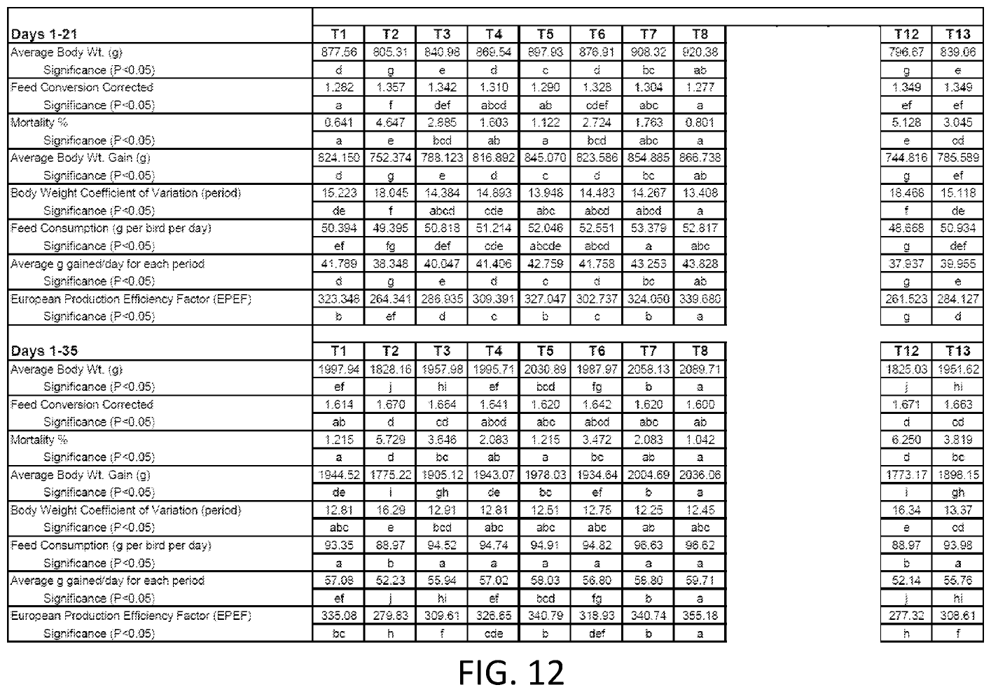

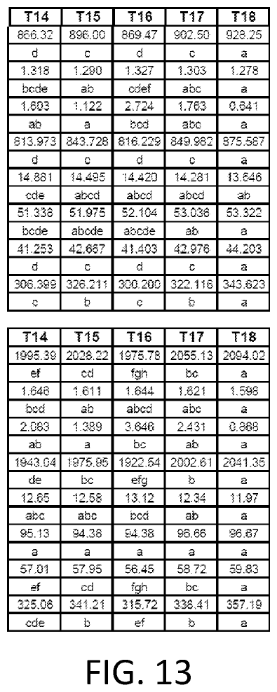

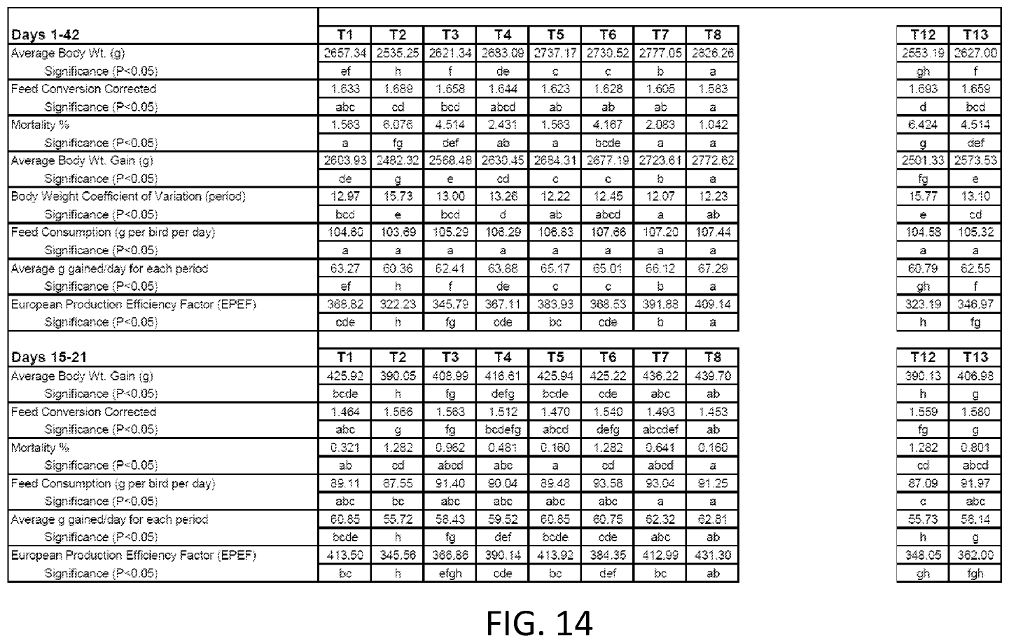

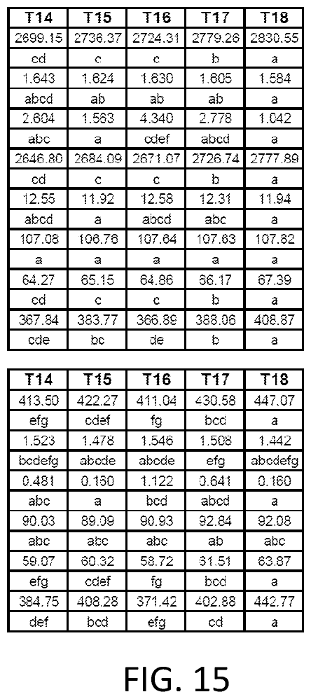

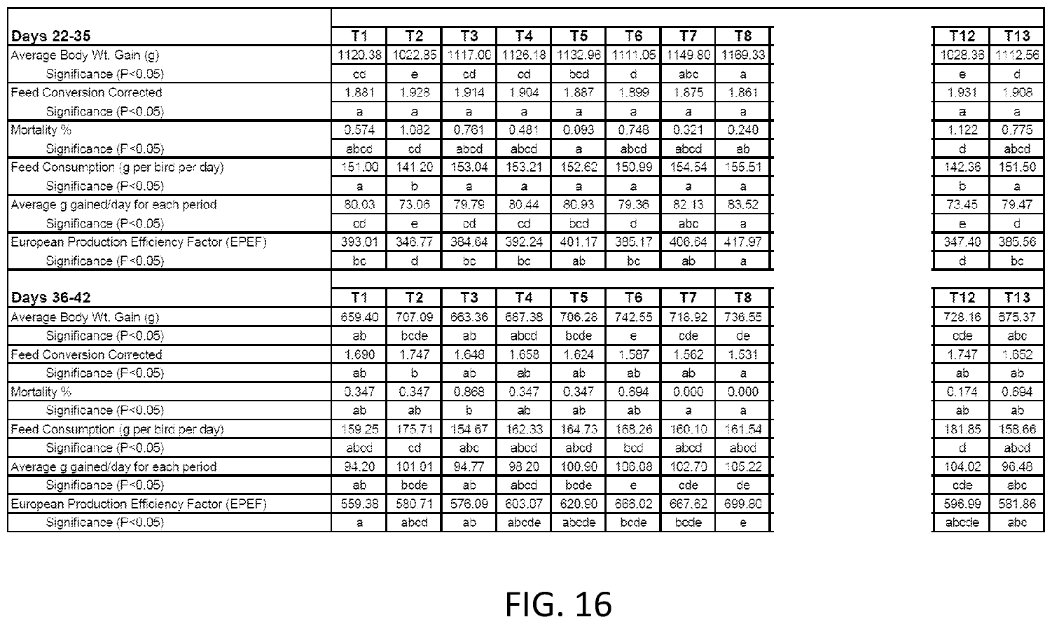

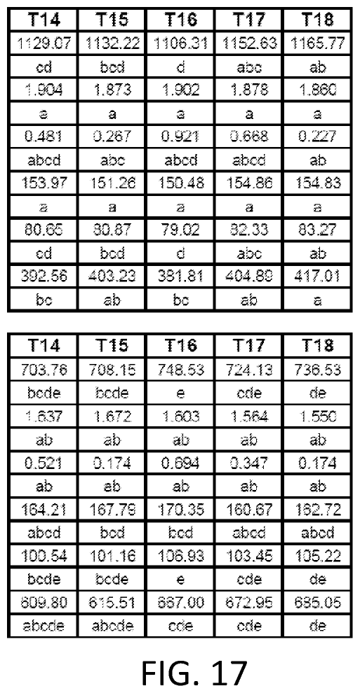

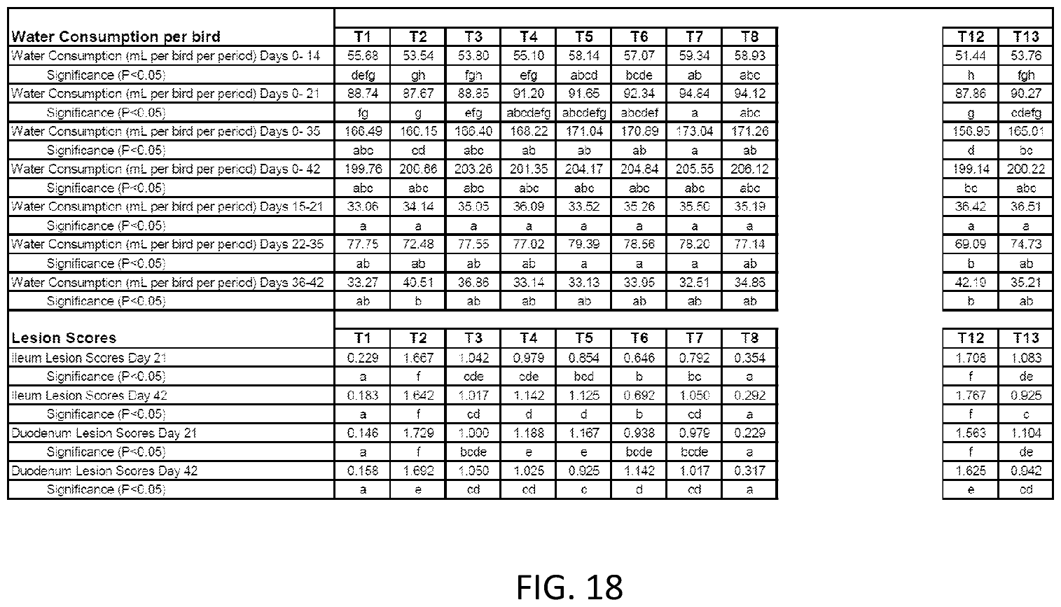

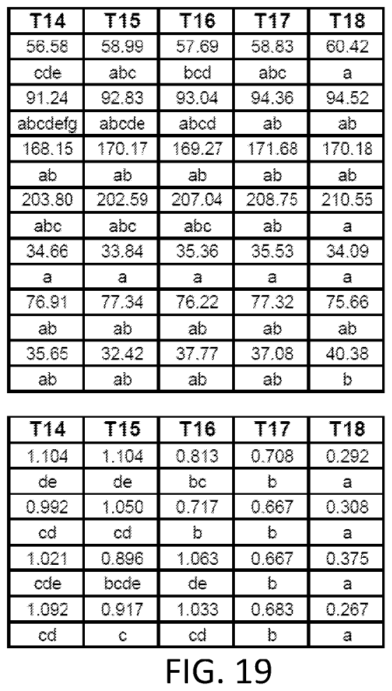

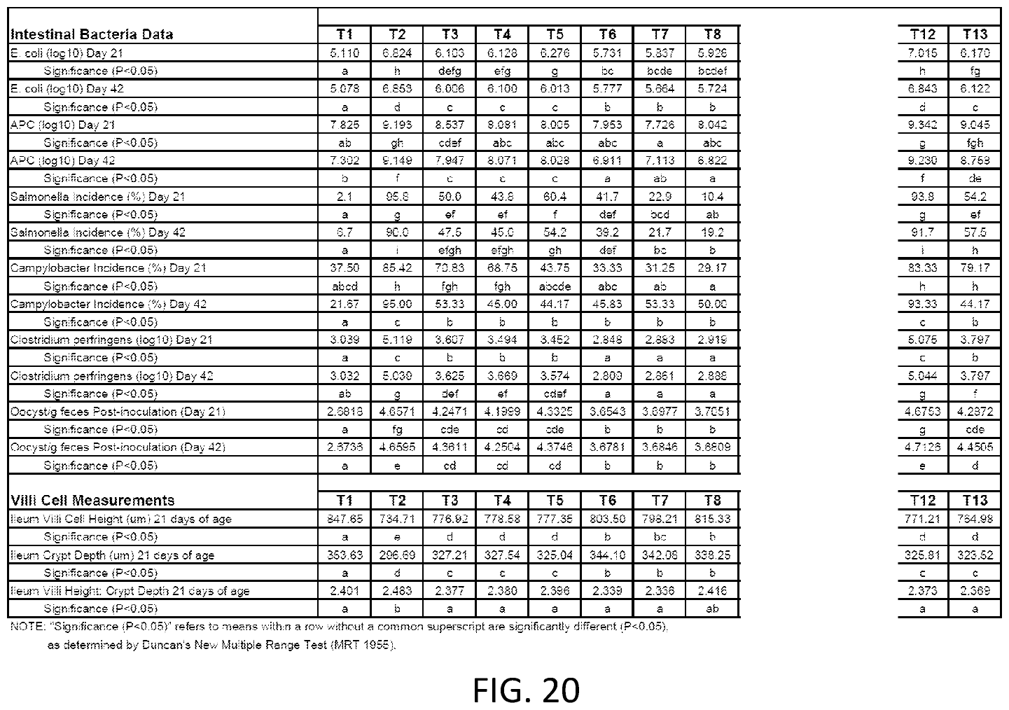

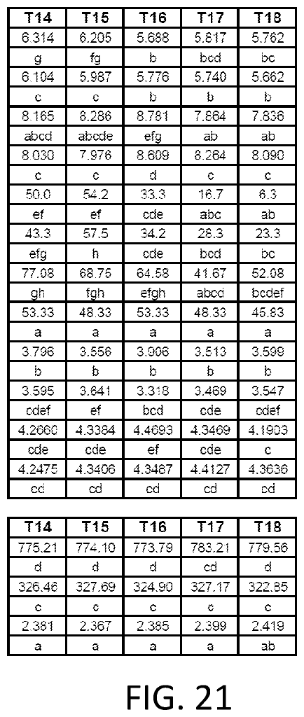

Results are shown in FIGS. 10-21.
CLINICAL OBSERVATIONS: Chicks were observed at least two times daily beginning on trial day 0 to determine mortality or the onset, severity, and duration of any behavioral changes or evidence of toxicity (including fecal material condition, presence of diarrhea, nervousness, accessibility to water and feed, general bird appearance, and any adverse conditions which could have affected performance).
HEALTH EXAMS: Health exams were conducted at 21 and 42 days of age (depending upon weight gains).
MEAN BODY WEIGHTS: Body weights were taken by weighing individual chicks in a pen and recorded for Trial Days 21 and 42 (depending upon weight gains). Body weight gain was calculated by determining actual body weight gain (ending minus beginning weights) during the periods of trial days 0-21, 22-42 and 0-42. Body weights were taken on both moribund birds and test animals that were found dead during the study.
BODY WEIGHT UNIFORMITY: Body weight uniformity (CV or Coefficient of Variation) was determined on Trial Days 21 and 42.
FEED CONSUMPTION: Feed weigh-backs were taken on Trial Days 0-21, 22-42 and 0-42. Food consumption was evaluated for each pen on trial days 0-21, 22-42 and 0-42. A separate container (feed trough) was assigned for each pen. The initial tare weight for each feed barrel were recorded on Trial Day 0. Feed was added and the weight recorded. When adding feed, the feed barrel was first weighed and the weight recorded (Weight out). Feed out means feed that is removed and taken out of the calculations. New feed was added and the weight recorded (Weight in). Any feed spilled was weighed then discarded.
FEED CONVERSION (weight: gain ratio): Feed Conversion was determined on Trial Days 0-21, 22-42 and 0-42.
MORTALITY: Mortality was taken daily and reported as percentage per time period for Trial Days 0-21, 22-42 and 0-42.
WATER CONSUMPTION: Water consumption was measured continuously during the course of the study.
INTESTINAL BACTERIA COUNTS: Intestinal Bacteria Counts including Clostridium perfringens, E. coli and APC (Aerobic Plate Counts) were taken at both 21 (2M and 2F from each pen) and 42 days of age (5M and 5F) from each pen.

Salmonella spp. (wild stain, both Salmonella typhimurium and Salmonella Heidelberg) Incidence per g GIT feces was determined at both 21 (2M and 2F from each pen) and 42 days of age (5M and 5F) from each pen.

Campylobacter spp. (wild stain) Incidence per g GIT feces was determined at both 21 (2M and 2F from each pen) and 42 days of age (5M and 5F) from each pen.

Villi Height and Crypt Depth were determined on Trial Days 21 and 42. Intestinal samples (from 2M and 2F birds) were taken from two gut areas per bird (one at the distal to the end of the duodenal loop or 1″ to 2″ below the end of the loop and the other sample in the second in the mid gut area or a few inches anterior to Meckel's diverticulum). Both villi height and crypt depth were determined on these two areas of the gut.

Tissue sample collection occurred on Trial Days 7, 14, 21, and 35. Intestinal samples (from 2M and 2F birds) were taken from two gut areas per bird (one at the distal to the end of the duodenal loop or 1″ to 2″ below the end of the loop and the other sample in the second in the mid gut area or a few inches anterior to Meckel's diverticulum). Both villi height and crypt depth were determined on these two areas of the gut.

Cecal contents was collected on day 42. One bird of average pen size from twelve replicate pens were euthanized per animal care guidelines for collection of cecal contents for microbiome analysis. Ceca were excised at the ileal-cecal junction and approximately 1 to 1.5 ml of cecal contents were transferred into 2 ml labeled cryovials on ice and stored at −80° C.

### VII. Exemplary Embodiments

The following numbered paragraphs illustrate exemplary embodiments of the disclosed technology.

Paragraph 1. A combination and/or composition comprising a Bacillus combination and an essential oil composition.

Paragraph 2. The combination and/or composition of paragraph 1, wherein the Bacillus combination comprises Bacillus amyloliquefaciens, Bacillus subtilis, Bacillus licheniformis and Bacillus coagulans.

Paragraph 3. The combination and/or composition of paragraph 1 or paragraph 2, wherein the Bacillus combination consists essentially of Bacillus amyloliquefaciens, Bacillus subtilis, Bacillus licheniformis and Bacillus coagulans.

Paragraph 4. The combination and/or composition of any one of paragraphs 1-3, wherein the essential oil composition comprises oregano essential oil.

Paragraph 5. The combination and/or composition of any one of paragraphs 1-3, wherein the essential oil composition consists essentially of oregano essential oil.

Paragraph 6. The combination and/or composition of any one of paragraphs 1-5, wherein the combination and/or composition further comprises yucca, quillaja, or a combination thereof.

Paragraph 7. The combination and/or composition of paragraph 6, wherein the yucca is Yucca schidigera, and the quillaja is Quillaja saponaria.

Paragraph 8. The combination and/or composition of paragraph 6 or paragraph 7, wherein the yucca and quillaja are present in amounts of from 95% quillaja:5% yucca to 80% quillaja:20% yucca relative to each other.

Paragraph 9. The combination and/or composition of paragraph 8, wherein the yucca and quillaja are present in amounts of from 95% quillaja:5% yucca to 90% quillaja:10% yucca relative to each other.

Paragraph 10. The combination and/or composition of any one of paragraphs 1-9, wherein the Bacillus combination consists essentially of from 30% to 45% Bacillus subtilis, from 40% to 60% Bacillus licheniformis, from 10% to 25% Bacillus amyloliquefaciens and from 1% to 12% Bacillus coagulans.

Paragraph 11. The combination and/or composition of any one of paragraphs 1-10, wherein the Bacillus combination further comprises a carrier.

Paragraph 12. The combination and/or composition of any one of paragraphs 1-11, further comprising an additional component.

Paragraph 13. The combination and/or composition of paragraph 12, wherein the additional component comprises silica, mineral clay, glucan, mannans, endoglucanohydrolase, a copper salt, a vitamin, a chromium compound, metal chelate, growth factor, allicin, alliin, alliinase, yeast, DFM, or a combination thereof.

Paragraph 14. A feed composition, comprising:
the combination and/or composition according to any one of paragraphs 1-13; and
an animal feed.

Paragraph 15. The feed composition of paragraph 14, wherein the feed composition comprises:
a Bacillus combination comprising Bacillus amyloliquefaciens, Bacillus subtilis, Bacillus licheniformis, Bacillus coagulans;
oregano essential oil; and
yucca and quillaja, or extracts thereof.

Paragraph 16. The feed composition according to paragraph 14 or paragraph 15, wherein the animal feed is a poultry feed.

Paragraph 17. The feed composition according to any one of paragraphs 14-16, wherein the feed composition comprises from 1×10⁵ to 1×10⁷ CFU of the Bacillus combination per gram of the feed.

Paragraph 18. The feed composition according to paragraph 17, wherein the feed composition comprises from 2.5×10⁵ to 5×10⁶ CFU of the Bacillus combination per gram of the feed.

Paragraph 19. The feed composition according to any one of paragraphs 14-16, wherein the feed composition comprises from 0.1 pounds to 1 pound of the Bacillus combination per ton of the feed.

20. The feed composition according to paragraph 19, wherein the feed composition comprises from 100 grams to 250 grams of the Bacillus combination per ton of the feed.

Paragraph 21. The feed composition of any one of paragraphs 14-20, wherein the feed composition comprises from the essential oil composition in an amount sufficient to provide from 25 ppm to 500 ppm of essential oil.

Paragraph 22. The feed composition of any one of paragraphs 14-20, wherein the feed composition comprises from the essential oil composition in an amount sufficient to provide from 50 grams to 250 grams of essential oil per ton of feed.

Paragraph 23. The feed composition of any one of paragraphs 14-22, wherein the essential oil composition comprises oregano essential oil.

Paragraph 24. The feed composition of any one of paragraphs 14-23, wherein the feed composition further comprises from 100 ppm to 500 ppm Yucca schidigera and Quillaja Saponaria or extracts thereof per ton of feed.

Paragraph 25. The feed composition of any one of paragraphs 14-23, wherein the feed composition further comprises from 100 grams to 250 grams Yucca schidigera and Quillaja Saponaria or extracts thereof per ton of feed.

Paragraph 26. The feed composition of paragraph 14, comprising:
from 5×10⁵ CFU to 1.2×10⁶ CFU Bacillus per gram of feed;
from 200 ppm to 400 ppm Yucca schidigera and Quillaja Saponaria per ton of feed; and
from 50 to 250 ppm oregano essential oil per ton of feed.

Paragraph 27. The feed composition of paragraph 14, comprising:
from 5×10⁵ CFU to 1.2×10⁶ CFU Bacillus per gram of feed;
from 110 grams to 115 grams Yucca schidigera and Quillaja Saponaria per ton of feed; and
from 100 grams to 300 grams oregano essential oil per ton of feed.

Paragraph 28. A method, comprising administering a combination and/or composition according to any one of paragraphs 1-13, or a feed composition according to any one of paragraphs 14-27 to a non-ruminant animal.

Paragraph 29. The method according to paragraph 28, wherein the non-ruminant animal is poultry.

Paragraph 30. The method according to paragraph 28 or paragraph 29, wherein administration of the combination and/or composition, or the feed composition, increases breast meat yield, increases weight gain, improves a feed conversion ratio, reduces bird mortality, reduces lesion scores, reduces Salmonella/E. Coli/Clostridium perfringens (CP) incidence, reduces oocysts in fecal matter, or a combination thereof.

In view of the many possible embodiments to which the principles of the disclosure may be applied, it should be recognized that the illustrated embodiments are only preferred examples of the invention and should not be taken as limiting the scope of the invention. Rather, the scope of the disclosure is defined by the following claims. We therefore claim as our invention all that comes within the scope and spirit of these claims.

I claim:

1. A combination and/or composition comprising:
a Bacillus combination comprising Bacillus amyloliquefaciens, Bacillus subtilis, Bacillus licheniformis and Bacillus coagulans;
yucca, quillaja, or a combination thereof; and
oregano essential oil.

2. The combination and/or composition of claim 1, wherein the Bacillus combination consists essentially of Bacillus amyloliquefaciens, Bacillus subtilis, Bacillus licheniformis and Bacillus coagulans.

3. The combination and/or composition of claim 1, wherein the yucca is Yucca schidigera, and the quillaja is Quillaja saponaria.

4. The combination and/or composition of claim 1, wherein the yucca and quillaja are present in amounts of from 95% quillaja:5% yucca to 80% quillaja:20% yucca relative to each other.

5. The combination and/or composition of claim 1, wherein the Bacillus combination consists essentially of from 30% to 45% Bacillus subtilis, from 40% to 60% Bacillus licheniformis, from 10% to 25% Bacillus amyloliquefaciens and from 1% to 12% Bacillus coagulans.

6. The combination and/or composition of claim 1, further comprising an additional component.

7. The combination and/or composition of claim 6, wherein the additional component comprises silica, mineral clay, glucan, mannans, endoglucanohydrolase, a copper salt, a vitamin, a chromium compound, metal chelate, growth factor, allicin, alliin, alliinase, yeast, DFM, or a combination thereof.

8. The combination and/or composition of claim 1, wherein the combination and/or composition comprises
a Bacillus combination consists essentially of from 30% to 45% Bacillus subtilis, from 40% to 60% Bacillus licheniformis, from 10% to 25% Bacillus amyloliquefaciens and from 1% to 12% Bacillus coagulans, and no additional Bacillus species;
Yucca schidigera or an extract thereof, and Quillaja saponaria or an extract thereof; and
oregano essential oil.

9. A feed composition, comprising:
the combination and/or composition according to claim 1; and
an animal feed.

10. The feed composition according to claim 9, wherein the animal feed is a poultry feed.

11. The feed composition according to claim 9, wherein the feed composition comprises from 1×10⁵ to 1×10⁷ CFU of the Bacillus combination per gram of the feed.

12. The feed composition according to claim 9, wherein the feed composition comprises from 0.1 pounds to 1 pound of the Bacillus combination per ton of the feed.

13. The feed composition of claim 9, wherein the feed composition comprises from the oregano essential oil composition in an amount sufficient to provide from 25 ppm to 500 ppm of essential oil.

14. The feed composition of claim 9, wherein the feed composition comprises from the essential oil composition in an amount sufficient to provide from 50 grams to 250 grams of essential oil per ton of feed.

15. The feed composition of claim 9, wherein the feed composition comprises from 100 ppm to 500 ppm Yucca schidigera and Quillaja Saponaria or extracts thereof per ton of feed.

16. The feed composition of claim 9, comprising:
from 5×10⁵ CFU to 1.2×10⁶ CFU Bacillus per gram of feed;
from 200 ppm to 400 ppm Yucca schidigera and Quillaja Saponaria per ton of feed; and
from 50 to 250 ppm oregano essential oil per ton of feed.

17. The feed composition of claim 9, comprising:
from 5×10⁵ CFU to 1.2×10⁶ CFU Bacillus per gram of feed;
from 110 grams to 115 grams Yucca schidigera and Quillaja Saponaria per ton of feed; and
from 100 grams to 300 grams oregano essential oil per ton of feed.

18. A method, comprising administering a combination and/or composition according to claim 1 to a non-ruminant animal.

19. The method according to claim 18, wherein the non-ruminant animal is poultry.

20. The method according to claim 19, wherein administration of the combination and/or composition, increases breast meat yield, increases weight gain, improves a feed conversion ratio, reduces bird mortality, reduces lesion scores, reduces Salmonella/E. Coli/Clostridium perfringens (CP) incidence, reduces oocysts in fecal matter, or a combination thereof.
